SEQUENCE LISTING (74 sequences)

SEQ ID NO: 1 - DNA, 2640 bp, Artificial Sequence
  Synthetic Construct
actagtgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagggtacccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagtctagaacgctaagtcggaggacggacggtcaggtactagcggcggtgtctagtttgctcttgccatcaacaatgcgtgccatgccttttctcgaatgtattttacaatttctgaagacgtcgggattggaaatcccaaagtattaataagcacattgtttataagactcgcatgtatgttaatactgtggatccgtgagtttctattcgcagtcggctgatctgtgtgaaatcttaataaagggtccaattaccaatttgaaactcaggaattcacagtattaacatacatgcgagtcttataaacaatgtgcttattaatactttgggatttccaatcccgacgtcttcagaaattgtaaaatacattcgagaaaaggcatggcacgcattgttgatggcaagagcaaactagacaccgccgctagtacctgaccgtccgtcctccgacttagcgtaagctttcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgtcgacggacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcatctcgtactcggcgtggaaccgccggctgaccgtcgaggacatcgaggtcgccccggagcaccgggggcacggggtcgggcgcgcgttgatggggctcgcgacggagttcgccggcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcggcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactggtcgaccaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagaggagctc

SEQ ID NO: 2 - DNA, 8610 bp, Artificial Sequence
  Synthetic Construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgtatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatactagtgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagggtacccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagtctagaacgctaagtcggaggacggacggtcaggtactagcggcggtgtctagtttgctcttgccatcaacaatgcgtgccatgccttttctcgaatgtattttacaatttctgaagacgtcgggattggaaatcccaaagtattaataagcacattgtttataagactcgcatgtatgttaatactgtggatccgtgagtttctattcgcagtcggctgatctgtgtgaaatcttaataaagggtccaattaccaatttgaaactcaggaattcacagtattaacatacatgcgagtcttataaacaatgtgcttattaatactttgggatttccaatcccgacgtcttcagaaattgtaaaatacattcgagaaaaggcatggcacgcattgttgatggcaagagcaaactagacaccgccgctagtacctgaccgtccgtcctccgacttagcgtaagctttcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgtcgacggacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcatctcgtactcggcgtggaaccgccggctgaccgtcgaggacatcgaggtcgccccggagcaccgggggcacggggtcgggcgcgcgttgatggggctcgcgacggagttcgccggcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcggcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactggtcgaccaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagaggagctcatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatctaagaaaccattattatcatgacattaacctataaaaataggcgtatcacgaggccctttcgtc

SEQ ID NO: 3 - DNA, 53 bp, Artificial Sequence
  Synthetic Construct
ggtcgacggtatcgataagcttgatgtatacgtgtgcccaatagaaagagaac

SEQ ID NO: 4 - DNA, 53 bp, Artificial Sequence
  Synthetic Construct
ggagtagaaacattttgaagctatgggtaccctccatgcagttggacgatatc

SEQ ID NO: 5 - DNA, 52 bp, Artificial Sequence
  Synthetic Construct
tgggacgctcgaaggctttaatttgcgtcgacgacatggaggcccagaatac

SEQ ID NO: 6 - DNA, 33 bp, Artificial Sequence
  Synthetic Construct
cttggtattcttgcagtatagcgaccagcattc

SEQ ID NO: 7 - DNA, 35 bp, Artificial Sequence
  Synthetic Construct
ggtcgctatactgcaagaataccaagagttcctcg

SEQ ID NO: 8 - DNA, 50 bp, Artificial Sequence
  Synthetic Construct
atcccccgggctgcaggaattcgatgtatacctcttgccttccaacccag

SEQ ID NO: 9 - DNA, 24 bp, Saccharomyces cerevisiae
gatcatttatctttcactgcggag

SEQ ID NO: 10 - DNA, 43 bp, Saccharomyces cerevisiae
aactgcatggagatgagtcggttttagagctagaaatagcaag

SEQ ID NO: 11 - DNA, 72 bp, Artificial Sequence
  Synthetic Construct
gtcaagaaagacactaagaacacagaaaagaaacacgaagagcagaggaaatgacatggaggcccagaatac

SEQ ID NO: 12 - DNA, 73 bp, Artificial Sequence
  Synthetic Construct
gggaagttttccaaatggcatgattactctatacagctgataaactctgtccagtatagcgaccagcattcac

SEQ ID NO: 13 - DNA, 21 bp, Saccharomyces cerevisiae
cctgcgtaagttggttaaacg

SEQ ID NO: 14 - DNA, 70 bp, Artificial Sequence
  Synthetic Construct
gattacaagataaaaaagccactactacagaaaaggcgttgggtcaggacgacatggaggcccagaatac

SEQ ID NO: 15 - DNA, 74 bp, Artificial Sequence
  Synthetic Construct
gctttattatctctctcctttctattcctcttttctctactgccctttttctcagtatagcgaccagcattcac

SEQ ID NO: 16 - DNA, 21 bp, Saccharomyces cerevisiae
gataaaaaggctctccatggc

SEQ ID NO: 17 - DNA, 72 bp, Artificial Sequence
  Synthetic Construct
gcctacagtagaaatcgattaatataaacatatatctagcaacgtaacggaggacatggaggcccagaatac

SEQ ID NO: 18 - DNA, 73 bp, Artificial Sequence
  Synthetic Construct
ctcacatcacctttaatcattttttcactcatgtaccagtatacgtcgacccagtatagcgaccagcattcac

SEQ ID NO: 19 - DNA, 21 bp, Saccharomyces cerevisiae
cgaattctcgtgcagtttttc

SEQ ID NO: 20 - DNA, 70 bp, Artificial Sequence
  Synthetic Construct
gatagacgaaataggaacaacaaacagcttataagcacccaataagtgcggacatggaggcccagaatac

SEQ ID NO: 21 - DNA, 74 bp, Artificial Sequence
  Synthetic Construct
gcatgggggagccataactccatgacacagatattcgattagatgaatttagcagtatagcgaccagcattcac

SEQ ID NO: 22 - DNA, 21 bp, Artificial Sequence
  Synthetic Construct
acccaaaaatatgagggcatc

SEQ ID NO: 23 - DNA, 72 bp, Artificial Sequence
  Synthetic Construct
gccacatagttctttccgatatgaacaacctaactcacaaaatttactgtacgacatggaggcccagaatac

SEQ ID NO: 24 - DNA, 73 bp, Artificial Sequence
  Synthetic Construct
ctatgtatacgtgtgtgtgtgtgtgtgcaataagagttcgaaaacattaaccagtatagcgaccagcattcac

SEQ ID NO: 25 - DNA, 21 bp, Saccharomyces cerevisiae
atcacgacggacgaagtattg

SEQ ID NO: 26 - DNA, 22 bp, Artificial Sequence
  Synthetic Construct
cccatataaatcagcatccatg

SEQ ID NO: 27 - DNA, 21 bp, Artificial Sequence
  Synthetic Construct
cgatcagaaacttctcgacag

SEQ ID NO: 28 - DNA, 22 bp, Saccharomyces cerevisiae
cgtctggattggtggttctatc

SEQ ID NO: 29 - DNA, 22 bp, Saccharomyces cerevisiae
ggaccactttcgtcgtattctt

SEQ ID NO: 30 - DNA, 29 bp, Saccharomyces cerevisiae
cacggatagtggctttggtgaacaattac

SEQ ID NO: 31 - DNA, 32 bp, Saccharomyces cerevisiae
tatgattatctggcagcaggaaagaacttggg

SEQ ID NO: 32 - DNA, 486 bp, Drosophila melanogaster
agttattccgtttggcagcaaaaaatctccgaatctgaaacaaatggtctgcattgattgaaaatacaatttgctgactattcttggtcaaagaatgcgcaaatgtttgattatgtcagacactttggcatagcatagaaattgaaaatatcatatcaaatattattgtttaaatgttcgatctttaagggtaatcattgggatccgtgagtttctattcgcagtcggctgatctgtgtgaaatcttaataaagggtccaattaccaatttgaaactcaggaattccaatgattacccttaaagatcgaacatttaaacaataatatttgatatgatattttcaatttctatgctatgccaaagtgtctgacataatcaaacatttgcgcattctttgaccaagaatagtcagcaaattgtattttcaatcaatgcagaccatttgtttcagattcggagattttttgctgccaaacggaataact

SEQ ID NO: 33 - DNA, 498 bp, Drosophila melanogaster
ttaacttggagcccgacaacgtcggtgttgtggtcttcggtaacgataagctgatcaagcagggcgatatcgtcaagcgtaccggtgccatcgtggatgtgcccgtcggtgatgagctgctgggtcgcgtcgtcgatgccctgggaaatgccatcgacggcaagggtgccatcaacaccaaggaccgtttccgtgtgggaatcaagggatccgtgagtttctattcgcagtcggctgatctgtgtgaaatcttaataaagggtccaattaccaatttgaaactcaggaattccttgattcccacacggaaacggtccttggtgttgatggcacccttgccgtcgatggcatttcccagggcatcgacgacgcgacccagcagctcatcaccgacgggcacatccacgatggcaccggtacgcttgacgatatcgccctgcttgatcagcttatcgttaccgaagaccacaacaccgacgttgtcgggctccaagttaa

SEQ ID NO: 34 - DNA, 486 bp, Aedes aegypti
ctccgaagatgaggccgttgctaacgatttggatatgcacgcattgattctgggcggccttcacactgacaatgatccgataaagacagcggaagaggtcatcaaggaaattgacgatattatggacgaaagcgcctccgaagacggcattgttggtaacgaaatcatggaaaaagccaaagaagttcttggatctccccggatccgtgagtttctattcgcagtcggctgatctgtgtgaaatcttaataaagggtccaattaccaatttgaaactcaggaattcggggagatccaagaacttctttggctttttccatgatttcgttaccaacaatgccgtcttcggaggcgctttcgtccataatatcgtcaatttccttgatgacctcttccgctgtctttatcggatcattgtcagtgtgaaggccgcccagaatcaatgcgtgcatatccaaatcgttagcaacggcctcatcttcggag

SEQ ID NO: 35 - DNA, 486 bp, Aedes aegypti
cctgcagatgcgctgcgagaagctggtcgaagaacgcgatcagctgaagaatatgttcgagaagtctatactggagctgcaacagaagtcaggtttgaaaaattccttattggagcgaaaactagaatacatcgagaagcaaacggaacaacgggaagccattttaggggaggtgttatcgcttgccggaatcgaaccgcggatccgtgagtttctattcgcagtcggctgatctgtgtgaaatcttaataaagggtccaattaccaatttgaaactcaggaattcgcggttcgattccggcaagcgataacacctcccctaaaatggcttcccgttgttccgtttgcttctcgatgtattctagttttcgctccaataaggaatttttcaaacctgacttctgttgcagctccagtatagacttctcgaacatattcttcagctgatcgcgttcttcgaccagcttctcgcagcgcatctgcagg

SEQ ID NO: 36 - DNA, 486 bp, Aedes aegypti
cgagcatttcagcgataactttcatacctatggacttgtgtggaagccggacagcatcgctctgaccgtggatggattccagtatgctaccctgagggatcggttcaagccgtacggtgcggccaacaatttgacccaggcgaatttgtggaatccggacaatgccatgtcaccgtttgatcgagagttttacatatcgcggatccgtgagtttctattcgcagtcggctgatctgtgtgaaatcttaataaagggtccaattaccaatttgaaactcaggaattcgcgatatgtaaaactctcgatcaaacggtgacatggcattgtccggattccacaaattcgcctgggtcaaattgttggccgcaccgtacggcttgaaccgatccctcagggtagcatactggaatccatccacggtcagagcgatgctgtccggcttccacacaagtccataggtatgaaagttatcgctgaaatgctcg

SEQ ID NO: 37 - DNA, 486 bp, Aedes aegypti
cccggaaggagtgtacatggaagtggacgatgaagtgtactgtcatattgacccggaagaaggcttctacaacgaggtgaaagcgacgaaaccgcaatttgcaaacctttggagattgagcggtaatcgaatggctccgttcgataaggagttcttcattagtttgggcgtcggtgtgggtggtcactacgacttccaccggatccgtgagtttctattcgcagtcggctgatctgtgtgaaatcttaataaagggtccaattaccaatttgaaactcaggaattcggtggaagtcgtagtgaccacccacaccgacgcccaaactaatgaagaactccttatcgaacggagccattcgattaccgctcaatctccaaaggtttgcaaattgcggtttcgtcgctttcacctcgttgtagaagccttcttccgggtcaatatgacagtacacttcatcgtccacttccatgtacactccttccggg

SEQ ID NO: 38 - DNA, 486 bp, Aedes aegypti
aaccattgttgagcgatattatcattattacactagtgatcatattataacttattaacaaactatttgtagcgtagtgatgatggagagaggagtatcgaagaagaggcaggagaagcaagtcagataaatattaggaaagtatgcgaaaaacacgtgaataaaaaaaatacactactgatccgagtaacggtagctggggatccgtgagtttctattcgcagtcggctgatctgtgtgaaatcttaataaagggtccaattaccaatttgaaactcaggaattcccagctaccgttactcggatcagtagtgtattttttttattcacgtgtttttcgcatactttcctaatatttatctgacttgcttctcctgcctcttcttcgatactcctctctccatcatcactacgctacaaatagtttgttaataagttataatatgatcactagtgtaataatgataatatcgctcaacaatggtt

SEQ ID NO: 39 - DNA, 486 bp, Aedes aegypti
tgaaactcttacgtgtatcgacggttcttgggacagttcagtgtttcgatgtgagcccacctgtggaacaccaacgccagatgctgaagcatacattattggaggtcgaaatgccaccataacggaggtcccatggcatactggaatatatcgaaatctggaaacagacaccatcgaagatcttcgatcagaagattggcggatccgtgagtttctattcgcagtcggctgatctgtgtgaaatcttaataaagggtccaattaccaatttgaaactcaggaattcgccaatcttctgatcgaagatcttcgatggtgtctgtttccagatttcgatatattccagtatgccatgggacctccgttatggtggcatttcgacctccaataatgtatgcttcagcatctggcgttggtgttccacaggtgggctcacatcgaaacactgaactgtcccaagaaccgtcgatacacgtaagagtttca

SEQ ID NO: 40 - DNA, 486 bp, Mus musculus
tgaactcaactgtgaaatgccaccttttgacagtgatgagaatgacctgttctttgaagttgacggaccccaaaagatgaagggctgcttccaaacctttgacctgggctgtcctgatgagagcatccagcttcaaatctcgcagcagcacatcaacaagagcttcaggcaggcagtatcactcattgtggctgtggagaggatccgtgagtttctattcgcagtcggctgatctgtgtgaaatcttaataaagggtccaattaccaatttgaaactcaggaattctctccacagccacaatgagtgatactgcctgcctgaagctcttgttgatgtgctgctgcgagatttgaagctggatgctctcatcaggacagcccaggtcaaaggtttggaagcagcccttcatcttttggggtccgtcaacttcaaagaacaggtcattctcatcactgtcaaaaggtggcatttcacagttgagttca

SEQ ID NO: 41 - DNA, 486 bp, Mus musculus
gctccgagatgaacaacaaaaaagcctcgtgctgtcggacccatatgagctgaaagctctccacctcaatggacagaatatcaaccaacaagtgatattctccatgagctttgtacaaggagaaccaagcaacgacaaaatacctgtggccttgggcctcaaaggaaagaatctatacctgtcctgtgtaatgaaagacgggatccgtgagtttctattcgcagtcggctgatctgtgtgaaatcttaataaagggtccaattaccaatttgaaactcaggaattccgtctttcattacacaggacaggtatagattctttcctttgaggcccaaggccacaggtattttgtcgttgcttggttctccttgtacaaagctcatggagaatatcacttgttggttgatattctgtccattgaggtggagagctttcagctcatatgggtccgacagcacgaggcttttttgttgttcatctcggagc

SEQ ID NO: 42 - DNA, 486 bp, Mus musculus
gtcttcctgggaaacaacagtggtcaggacataattgacttcaccatggaatccgtgtcttcctaaagtatgggctggactgtttctaatgccttccccagggcatgttaaggagctcccttttcgtgaatgagcagacagctcaatctccaggggactccttagtcctcggccaagacaggtcgctcagggtcacaagaggatccgtgagtttctattcgcagtcggctgatctgtgtgaaatcttaataaagggtccaattaccaatttgaaactcaggaattctcttgtgaccctgagcgacctgtcttggccgaggactaaggagtcccctggagattgagctgtctgctcattcacgaaaagggagctccttaacatgccctggggaaggcattagaaacagtccagcccatactttaggaagacacggattccatggtgaagtcaattatgtcctgaccactgttgtttcccaggaagac

SEQ ID NO: 43 - DNA, 492 bp, Artificial Sequence
  Synthetic Construct
cggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaagggatccgtgagtttctattcgcagtcggctgatctgtgtgaaatcttaataaagggtccaattaccaatttgaaactcaggaattccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccg

SEQ ID NO: 44 - DNA, 6816 bp, Artificial Sequence
  Synthetic Construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgtatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagtctagaacgctaagtcggaggacggacggtcaggtactagcggcggtgtctagtttgctcttgccatcaacaatgcgtgccatgccttttctcgaatgtattttacaatttctgaagacgtcgggattggaaatcccaaagtattaataagcacattgtttataagactcgcatgtatgttaatactgtggatccgtgagtttctattcgcagtcggctgatctgtgtgaaatcttaataaagggtccaattaccaatttgaaactcaggaattcacagtattaacatacatgcgagtcttataaacaatgtgcttattaatactttgggatttccaatcccgacgtcttcagaaattgtaaaatacattcgagaaaaggcatggcacgcattgttgatggcaagagcaaactagacaccgccgctagtacctgaccgtccgtcctccgacttagcgtaagctttcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatctaagaaaccattattatcatgacattaacctataaaaataggcgtatcacgaggccctttcgtc

SEQ ID NO: 45 - DNA, 2504 bp, Artificial Sequence
  Synthetic Construct
cgtcgacggtatcgataagcttgatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagctcggtacccgagttaaagatctgccaattgaacataacatggtagttacatatactagtaatatggttcggcacacattaaaagtataaaaactatctgaattacgaattacatatattggtcataaaaatcaatcaatcatcgtgtgttttatatgtctcttatctaagtataagaatatccatagttaatattcacttacgctaccttttaacctgtaatcattgtcaacaggatatgttaacgacccacattgataaacgctagtatttctttttcctcttcttattggccggctgtctctatactcccctatagtctgtttcttttcgtttcgattgttttacgtttgaggcctcgtggcgcacatggtacgctgtggtgctcgcggctgggaacgaaactctgggagctgcgattggcagcaatctaatctaagtctagaacgctaagtcggaggacggacggtcaggtactagcggcggtgtctagtttgctcttgccatcaacaatgcgtgccatgccttttctcgaatgtattttacaatttctgaagacgtcgggattggaaatcccaaagtattaataagcacattgtttataagactcgcatgtatgttaatactgtggatccgtgagtttctattcgcagtcggctgatctgtgtgaaatcttaataaagggtccaattaccaatttgaaactcaggaattcacagtattaacatacatgcgagtcttataaacaatgtgcttattaatactttgggatttccaatcccgacgtcttcagaaattgtaaaatacattcgagaaaaggcatggcacgcattgttgatggcaagagcaaactagacaccgccgctagtacctgaccgtccgtcctccgacttagcgtaagctttcatgtccatatccaacttccaatttaatctttcttttttaattttcacttatttgcgatacagaaagaggggatccgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcatctcgtactcggcgtggaaccgccggctgaccgtcgaggacatcgaggtcgccccggagcaccgggggcacggggtcgggcgcgcgttgatggggctcgcgacggagttcgccggcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcggcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagag

SEQ ID NO: 46 - DNA, 6851 bp, Artificial Sequence
  Synthetic Construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgtatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggcccccggttcgattccgggcttgcgcatcttttttactttatatactattttttttttttttctttttcccaaattttttcatgaaaaatttggcggaacggtacataagaatagaagagattcgttatgaaaattttctactctctttcacattttttttttcataagaattaaaaaaattctagaacgctaagtcggaggacggacggtcaggtactagcggcggtgtctagtttgctcttgccatcaacaatgcgtgccatgccttttctcgaatgtattttacaatttctgaagacgtcgggattggaaatcccaaagtattaataagcacattgtttataagactcgcatgtatgttaatactgtggatccgtgagtttctattcgcagtcggctgatctgtgtgaaatcttaataaagggtccaattaccaatttgaaactcaggaattcacagtattaacatacatgcgagtcttataaacaatgtgcttattaatactttgggatttccaatcccgacgtcttcagaaattgtaaaatacattcgagaaaaggcatggcacgcattgttgatggcaagagcaaactagacaccgccgctagtacctgaccgtccgtcctccgacttagcgtaagctttcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatctaagaaaccattattatcatgacattaacctataaaaataggcgtatcacgaggccctttcgtc

SEQ ID NO: 47 - DNA, 23 bp, Mus musculus
tgaactcaactgtgaaatgccac

SEQ ID NO: 48 - DNA, 20 bp, Mus musculus
tcatcaggacagcccaggtc

SEQ ID NO: 49 - DNA, 22 bp, Mus musculus
gtacaaggagaaccaagcaacg

SEQ ID NO: 50 - DNA, 23 bp, Mus musculus
taagatttcacacagatcagccg

SEQ ID NO: 51 - DNA, 22 bp, Mus musculus
ggaaacaacagtggtcaggaca

SEQ ID NO: 52 - DNA, 21 bp, Mus musculus
aggagtcccctggagattgag

SEQ ID NO: 53 - DNA, 23 bp, Drosophila melanogaster
ccgaatctgaaacaaatggtctg

SEQ ID NO: 54 - DNA, 25 bp, Drosophila melanogaster
ctatgccaaagtgtctgacataatc

SEQ ID NO: 55 - DNA, 22 bp, Drosophila melanogaster
tgtggtcttcggtaacgataag

SEQ ID NO: 56 - DNA, 18 bp, Drosophila melanogaster
gacccagcagctcatcac

SEQ ID NO: 57 - DNA, 20 bp, Artificial Sequence
  Synthetic Construct
gctgaccctgaagttcatct

SEQ ID NO: 58 - DNA, 19 bp, Artificial Sequence
  Synthetic Construct
agaagtcgtgctgcttcat

SEQ ID NO: 59 - DNA, 22 bp, Aedes aegypti
gacctcttccgctgtctttatc

SEQ ID NO: 60 - DNA, 25 bp, Aedes aegypti
cgttactcggatcagtagtgtattt

SEQ ID NO: 61 - DNA, 21 bp, Aedes aegypti
atactggagctgcaacagaag

SEQ ID NO: 62 - DNA, 22 bp, Aedes aegypti
gacctcttccgctgtctttatc

SEQ ID NO: 63 - DNA, 20 bp, Aedes aegypti
gaagatgaggccgttgctaa

SEQ ID NO: 64 - DNA, 22 bp, Aedes aegypti
gacctcttccgctgtctttatc

SEQ ID NO: 65 - DNA, 22 bp, Aedes aegypti
cgagcatttcagcgataacttt

SEQ ID NO: 66 - DNA, 22 bp, Aedes aegypti
ctcagggtagcatactggaatc

SEQ ID NO: 67 - DNA, 22 bp, Aedes aegypti
tgtcatattgacccggaagaag

SEQ ID NO: 68 - DNA, 20 bp, Aedes aegypti
cgaacggagccattcgatta

SEQ ID NO: 69 - DNA, 22 bp, Aedes aegypti
cttacgtgtatcgacggttctt

SEQ ID NO: 70 - DNA, 21 bp, Aedes aegypti
tggcatttcgacctccaataa

SEQ ID NO: 71 - DNA, 20 bp, Drosophila suzukii
acggacggtcaggtactagc

SEQ ID NO: 72 - DNA, 24 bp, Drosophila suzukii
tgcgagtcttataaacaatgtgct

SEQ ID NO: 73 - DNA, 19 bp, Saccharomyces cerevisiae
cagtgaaactgcgaatggc

SEQ ID NO: 74 - DNA, 23 bp, Saccharomyces cerevisiae
gaatcatcaaagagtccgaagac

(19) United States
(12) Patent Application Publication
(10) Pub. No.: US 2021/0054379 A1
(43) Pub. Date: Feb. 25, 2021

(54) YEAST FOR PRODUCING AND DELIVERING RNA BIOACTIVE MOLECULES AND METHODS AND USES THEREOF

(71) Applicant: RENAISSANCE BIOSCIENCE CORP., Vancouver (CA)

(72) Inventors: Ye Wang, Vancouver (CA); Jason Ken-Shun HUNG, Vancouver (CA); John Ivan HUSNIK, Vancouver (CA); Matthew S. DAHABIEH, Vancouver (CA); Hao DING, Vancouver (CA); Christopher SNOWDON, Vancouver (CA); Cedric Arthur BRIMACOMBE, Vancouver (CA)

(21) Appl. No.: 17/091,564

(22) Filed: Nov. 6, 2020

Related U.S. Application Data
(63) Continuation-in-part of application No. PCT/CA2019/050610, filed on May 8, 2019.
(60) Provisional application No. 62/669,118, filed on May 9, 2018.

(51) Int. Cl. C12N 15/113; A01N 63/60; A01N 63/50; C12N 15/81; A61K 36/06

(52) U.S. Cl. CPC C12N 15/113; A01N 63/60; A01N 63/50; C12N 2310/531; A61K 36/06; C12N 2310/14; C12N 15/81

(57) ABSTRACT

The present disclosure provide modified yeast that produce increased quantities of RNA bioactive molecules and methods of producing the same. Also provided are methods and uses of the yeast for biocontrol and disease protection.

### CROSS-REFERENCE TO RELATED APPLICATIONS

This application is a continuation-in-part of PCT/CA2019/050610 filed on May 8, 2019, which claims the benefit of priority to U.S. Provisional Application No. 62/669,118 filed May 9, 2018, the contents of both of which are incorporated herein by reference in their entirety.

### INCORPORATION OF SEQUENCE LISTING

A computer readable form of the Sequence Listing “23756-P55666US01_SequenceListing.txt”, (56,418 bytes), submitted via EFS-WEB and created on Nov. 5, 2020, is herein incorporated by reference.

### FIELD

The present disclosure provides yeast that are capable of producing RNA bioactive molecules, including RNA interference molecules. Also provided are methods and uses of the yeast for delivering RNA bioactive molecules to a subject in need thereof.

### BACKGROUND

The world population is expected to grow by 38% to 11.2 billion by the end of the century and there is an urgent need to make sure that the number of people who are undernourished decreases from the 1.1 billion reported in 2009 (Butler, 2010). While agricultural output after the Green Revolution currently outpaces human consumption, this balance is likely unsustainable, especially as arable land is converted to residential, commercial and industrial properties and the shift in growing economically high-value but low efficiency crops and livestock, such as strawberries and beef.

To that end, the world applies roughly 6 million tons of pesticides (worth US$56 billion) and over 3,000 tons of antibiotics (worth US$ a17.9 billion) annually (Atwood & Paisley, 2017; Pagel & Gautier, 2012; Van Boeckel, Brower, & Gilbert, 2015). The major goal of these pesticides and antibiotics is to allow more intensive agricultural practices such as mono-culturing and combine harvesting for crops and feedlot farming for livestock. However even with the integration of pesticides and antibiotics, roughly 40% of crop productivity and 18% of livestock productivity is lost worldwide due to agricultural pests and diseases (Drummond, Lambert, & Smalley, 1981; Oerke, 2006).

In terms of pesticides, part of this is due to intrinsically inaccurate administration; for example, the most common way to apply pesticides is through aerial spraying (crop dusting) and it is estimated that only 0.003 to 0.0000001% of applied pesticides ever reaches its intended target pest (Pimentel & Burgess, 2012), leaving the bulk (99.99%) to impact the surrounding environment and food chain. Furthermore, for pesticides applied aerially, roughly 50-70% never even reaches the ground, instead becoming “spray drift” which then effects surrounding non-farm areas such as forests and rivers (Pimentel, 2005).

Antibiotics are also not without their downsides as well. Since the wide spread use of antibiotics in livestock started in the 1940s, more and more cases of reduced antibiotic efficacy have appeared, even at ever increasing dosages due to microorganisms adapting and gaining resistances. A prime example is Salmonella enterica Typhimurium DT104, originally an exotic bird disease that has now become an epidemic in cows, pigs, chickens and humans.

Wide spread use of pesticides and antibiotics has also had significant unintended consequences on the environment and species biodiversity. Indeed, pesticide run off has been found to decrease biodiversity in streams by 42% in Europe and 27% in Australia, with susceptible species including insects, fish, crustaceans and birds (Beketov & Kefford, 2013). Indirect routes of antibiotic transmission into the environment have also been discovered through antibiotic-laced livestock waste contaminating the water supply, including lakes and rivers used to water crops, thus effecting the environment and the human food supply chain at the same time.

### RNA Interference

Given the aforementioned negative environmental and health-related effects of pesticides and antibiotics, as well as the need for a continually expanding food supply to keep pace with the growing population, there is an unmet need for novel agricultural bio-control technologies. Indeed, innovations such as genetically modified crops, integrated crop management, biological pest control (i.e. insect predators), probiotics, plasmid vaccination, and RNA interference (RNAi) have been explored or implemented. Of these technologies, RNAi is an attractive technology as it is organism specific, non-toxic to the environment, and potentially immune to resistance.

The concept of using RNAi as a bio-control agent is not in itself new; in addition to winning the Nobel Prize in 2006, Andrew Fire and Craig Mello were also issued a patent (U.S. Pat. No. 6,506,559 B1) for RNAi which included “a method to inhibit expression of a target gene in an invertebrate organism . . . ” (Fire, Kostas, Montgomery, & Timmons, 2003). It is also well established that exogenous RNAi effector molecules can be administered by genetic engineering (direct or vector mediated) or through environmental applications, such as soaking, injection, and/or feeding, depending on the target organism (Joga, Zotti, & Smagghe, 2016). However, significant biological, commercial and technical limitations to RNAi and the U.S. Pat. No. 6,506,559 B1 patent in particular have made it difficult to use in commercial agriculture applications; current commercial application of RNAi in agricultural bio-control is generally directed towards specific targets modulating existing bio-control methodologies (such as knocking out Bt resistance in pest organisms) rather than as a platform in itself.

### SUMMARY

The present inventors have demonstrated that modification of one or more key gene regulators of RNA production, regulation and degradation have significantly improved the expression of heterologous RNA but not RNA generally. This disclosure has a wide range of applications including the fields of crop bio-pesticides, bio-control of invasive species, livestock and aquaculture disease prophylaxis and/or treatment and as a therapeutic for human diseases.

Accordingly, the present disclosure provides a yeast cell comprising an RNA instability gene(s) that is downregulated or inactivated and/or an RNA stability gene(s) that is upregulated or heterologously expressed; and at least one heterologous sequence that encodes an RNA bioactive molecule. In an embodiment, the heterologous sequence is integrated into the yeast genome. In another embodiment, the heterologous sequence is plasmid-based.

In an embodiment, the yeast is Saccharomyces, such as S. cerevisiae.

In an embodiment, the RNA bioactive molecule is an mRNA. In another embodiment, the RNA bioactive molecule is an RNAi effector molecule.

In one embodiment, the RNAi effector molecule is siRNA, miRNA, lhRNA, shRNA, dsRNA, or anti-sense RNA. In a particular embodiment, the RNAi effector molecule is dsRNA. In another embodiment, the RNAi effector molecule is long hairpin RNA (lhRNA).

The RNA stability gene may be any gene or combination of genes that increase production or stabilize RNA in the yeast. In an embodiment, two RNA stability genes are upregulated or heterologously expressed.

In an embodiment, the RNA stability gene is in an expression cassette that is integrated into the yeast genome. In another embodiment, the RNA stability gene is plasmid-based.

In an embodiment, the RNA stability gene that is upregulated or heterologously expressed comprises or consists of CCR4 or THP1. In another embodiment, the RNA stability gene that is upregulated or heterologously expressed comprises or consists of XRN1 or TAF1.

The RNA instability gene may be any gene or combination of genes in the yeast that decrease production, destabilize or degrade RNA. In an embodiment, the RNA instability gene that is downregulated or inactivated comprises or consists of APN1, DBR1, DCS1, EDC3, HBS1, HTZ1, IPK1, LRP1, MAK10, MAK3, MAK31, MKT1, MPP6, MRT4, NAM7, NMD2, PAP2, POP2, RNH1, RNH203, RPS28A, RRP6, SIR3, SKI2, SKI3, SKI7, SKIS, SLH1, TRF5, or UPF3. In one embodiment, the RNA instability gene comprises or consists of HBS1, IPK1, LRP1, MAK10, MAK3, MAK31, MPP6, NAM7, NMD2, RRP6, SKI2, SKI3, or SKI7. In a particular embodiment, the RNA instability gene comprises or consists of LRP1. In another particular embodiment, the RNA instability gene comprises or consists of RRP6. In yet another particular embodiment, the RNA instability gene comprises or consists of SKI3. In a further particular embodiment, the RNA instability gene comprises or consists of MAK10. In yet a further particular embodiment, the RNA instability gene comprises or consists of MPP6.

In an embodiment, two RNA instability genes are downregulated or inactivated.

In one embodiment, the RNA instability genes that are downregulated or inactivated in the yeast comprise or consist of RRP6 and SKI3. In another embodiment, the RNA instability genes that are downregulated or inactivated in the yeast comprise or consist of LRP1 and RRP6. In yet another embodiment, the RNA instability genes that are downregulated or inactivated in the yeast comprise or consist of LRP1 and MAK3. In a further embodiment, the RNA instability genes that are downregulated or inactivated in the yeast comprise or consist of LRP1 and SKI2. In yet a further embodiment, the RNA instability genes that are downregulated or inactivated in the yeast comprise or consist of SKI2 and SKI3. In an even further embodiment, the RNA instability genes that are downregulated or inactivated in the yeast comprise or consist of SKI3 and MAK3.

In an embodiment, the RNA instability gene is downregulated or inactivated by deletion of the RNA instability gene in the yeast genome. In another embodiment, the RNA instability gene is downregulated or inactivated by any modification that reduces or abolishes its function, such as truncation, introduction of a stop codon or by point mutation. In yet another embodiment, the yeast may heterologously express factors that degrade or otherwise inactivate the protein product of the RNA instability gene (e.g. a dominant negative allele).

In an embodiment, the mRNA bioactive molecule encodes a protein that is useful for the treatment of a disease and/or infection, optionally immune factors that negatively regulate infection, such as stimulatory cytokines for macrophages; a protein that is related to a protein deficiency; or a protein that can elicit an immune or vaccine response for prevention or treatment of disease and/or infection.

In an embodiment, the RNAi effector molecule targets a gene involved in survival, maturation, reproduction, aggressiveness, or virulence of pests, or other infectious organisms, such as parasites, fungi, bacteria or viruses. In another embodiment, the RNAi effector molecule targets a gene involved in promoting a disease state, for example, in livestock, plants or humans.

In an embodiment, the gene involved in survival, maturation, reproduction, aggressiveness, or virulence comprises or consists of actin VATPase, cytochrome P450, hemolin, hunchback, bellwether, fez2, bicoid, modsp, boule, gas8, gnbpa1, gnpba3, tubulin, Sac1, Irc, otk, neurexin IV or vitellogenin. In one embodiment, the gene involved in survival, maturation, reproduction, aggressiveness, or virulence comprises or consists of bellwether or fez2. In another embodiment, the gene involved in survival, maturation, reproduction, aggressiveness, or virulence comprises or consists of neurexin IV. In another embodiment, the gene involved in promoting a disease state comprises or consists of actin, VATPase, cytochrome p450, hemolin, hunchback, vitellogenin, VEGF, VEGFR1, DDIT4, KRT6A, RRM2, p53, LMP2, LMP7, MECL1, IL-1β or TNF-α. In one embodiment, the disease is inflammatory bowel disease and the disease promoting gene is IL-1β.

The present disclosure also provides a method of producing a yeast cell that produces an increased amount of RNA bioactive molecules, the method comprising downregulating or inactivating an RNA instability gene disclosed herein and/or upregulating or heterologously expressing an RNA stability gene disclosed herein; and expressing at least one heterologous sequence that encodes an RNA bioactive molecule disclosed herein. In an embodiment, the method comprises integrating the at least one heterologous sequence into the yeast genome. In another embodiment, the method comprises introducing at least one plasmid-based heterologous sequence into the yeast. In an embodiment, downregulating or inactivating the RNA instability gene comprises deleting the gene from the yeast genome. In another embodiment, downregulating or inactivating the RNA instability gene comprises modifying it to reduce or abolish its function, such as by truncation, introduction of a stop codon or by point mutation. In yet another embodiment, the yeast may heterologously express factors that degrade or otherwise inactivate the protein product of the RNA instability gene (e.g. a dominant negative allele).

Further provided herein is a method of biocontrol comprising exposing an unwanted organism to a yeast cell that produces increased amounts of an RNA bioactive molecule as disclosed herein, wherein the RNA bioactive molecule reduces the survival, maturation, reproduction, aggressiveness, or virulence of the unwanted organism.

In an embodiment, exposing the organism to the yeast cell comprises feeding the yeast cells to the unwanted organism, optionally fresh, semi-dry or dry yeast.

In one embodiment, the RNA bioactive molecule that reduces the survival, maturation, reproduction, aggressiveness, or virulence of the unwanted organism is an mRNA that encodes for a toxic factor or a negative regulatory factor in a host harboring the unwanted organism.

In another embodiment, the RNA bioactive molecule is an RNAi effector molecule that targets a gene in the unwanted organism that is responsible for survival, maturation, reproduction, aggressiveness, or virulence. In an embodiment, the unwanted organism is a pest, a bacteria, a virus, a fungus or a parasite.

In one embodiment, the unwanted organism is an agricultural pest, such as an insect, and the RNAi effector molecule targets and silences the expression of at least one gene required by the pest for survival, maturation, reproduction, aggressiveness, or virulence. In an embodiment, the unwanted organism is a mosquito or a fly.

In an embodiment, the gene required by the pest is actin, VATPase, cytochrome p450, hemolin, hunchback, bellwether, fez2, bicoid, modsp, boule, gas8, gnbpa1, gnpba3, tubulin, Sac1 Irc, otk, neurexin-IV or vitellogenin. In one embodiment, the gene required by the pest is bellwether or fez2. In another embodiment, the gene required by the pest is neurexin-IV.

Even further provided herein is a method of treating a disease comprising exposing a subject having the disease to a yeast cell that produces increased amounts of an RNA bioactive molecule as disclosed herein, wherein the RNA bioactive molecule treats the disease.

In an embodiment, exposing the subject to the yeast comprises feeding the yeast cells to the subject, optionally as fresh, semi-dry or dry yeast. In another embodiment, exposing the subject to the yeast comprises intravenously, intradermally, intramuscularly, or subcutaneously injecting the yeast cell in the subject. In another embodiment, exposing the subject to the yeast comprises topical application or spraying of a solution of the yeast on the subject.

In one embodiment, the RNA bioactive molecule is an mRNA that encodes a protein that is useful for the treatment of the disease, optionally immune factors that negatively regulate infection, such as stimulatory cytokines for macrophages; an mRNA that encodes a protein that is related to a protein deficiency; or an mRNA that encodes a protein that can elicit an immune response for prevention or treatment of the disease.

In another embodiment, the RNA bioactive molecule is an RNAi effector molecule that targets a disease promoting gene in the subject.

In an embodiment, the subject is a plant or animal, such as livestock, a companion animal or a human.

In one embodiment, the disease promoting gene comprises or consists of VATPase, cytochrome p450, hemolin, hunchback, vitellogenin, VEGF, VEGFR1, DDIT4, KRT6A, RRM2, p53, LMP2, LMP7, MECL1, IL-1β or TNF-α.

In one embodiment, the disease is inflammatory bowel disease and the disease promoting gene is IL-1β.

Also provided is a method of treating an infection in a subject comprising exposing a subject having the infection to a yeast cell that produces increased amount of an RNA bioactive molecule as disclosed herein, wherein the RNA bioactive molecule is useful for treatment of the invention.

In one embodiment, the RNA bioactive molecule is an mRNA that encodes a protein that is useful for the treatment of the infection, optionally immune factors that negatively regulate infection, such as stimulatory cytokines for macrophages; or an mRNA that encodes a protein that can elicit an immune response for prevention or treatment of the infection.

In another embodiment, the RNA bioactive molecule is an RNAi effector molecule as disclosed herein, wherein the RNAi effector molecule targets an organism causing the infection in the subject or targets a host factor that promotes the infection in the subject. In an embodiment, the organism causing the infection is a virus, fungus, parasite or bacteria.

Other features and advantages of the present disclosure will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF THE DRAWINGS

Embodiments are described below in relation to the drawings in which:

FIG. 1A and FIG. 1B show a schematic representation of the RNAi effector reporter construct and expression vector. FIG. 1A. Construct contains a RNAi effector stem loop sequence driven by the S. cerevisiae TEF1 promoter and CYC1 terminator signals. The construct also contains a nourseothricin resistance cassette (natMX6) and is flanked by trp1 homology arms for integration into the yeast genome. FIG. 1B. The reporter construct was then integrated into pRS423-KanMX expression vector using Gibson cloning.

FIG. 2 shows RNAi expression profile screening of RNA processing gene knock out strains. Total RNA from various single gene knockout strains containing the RNAi reporter construct was used as input for quantitative reverse transcriptase PCR. Reporter gene expression is given as fold change relative to the wildtype yeast (white bar) and normalized to the reference gene ACT1.

FIG. 3 shows RNAi reporter expression profiles of select RNA processing single mutant strains. Total RNA from various single gene knockout strains containing the RNAi reporter construct was used as input for quantitative reverse transcriptase PCR. Reporter gene expression is given as fold change relative to the wildtype yeast (white bar) and normalized to the reference gene ALG1. Statistics were calculated using one-way ANOVA with WT sample as control; * P<0.05, ** P<0.01, *** P<0.001, **** P<0.0001.

FIG. 4 shows RNAi reporter expression profiles of select RNA processing double mutant strains. Total RNA from various double gene knockout strains, plus single gene knockout controls, containing the RNAi reporter construct was used as input for quantitative reverse transcriptase PCR. Reporter gene expression is given as fold change relative to the wildtype yeast (white bar) and normalized to the reference gene ALG1. Statistics were calculated using one-way ANOVA with WT sample as control; * P<0.05, ** P<0.01, *** P<0.001, **** P<0.0001.

FIG. 5 shows RNAi reporter expression profiles of candidate RNA stability gene single knockout mutants. Total RNA from various single gene knockout strains containing the RNAi reporter construct was used as input for quantitative reverse transcriptase PCR. Reporter gene expression is given as fold change (mean) relative to the wildtype yeast (white bar) and normalized to the reference gene ALG9. Error bars represent standard error of the sample mean between triplicate samples. Statistics were calculated using two-tailed T-test between each individual sample and the wild type control: * P<0.05, ** P<0.01, *** P<0.001.

FIG. 6 shows RNAi reporter expression profiles of XRN1 and TAF1 overexpressing strains. Total RNA from wild type BY4742 yeast cells bearing an RNAi effector expression construct integrated into the TRP1 locus and overexpressing XRN1 or TAF1 was used as input for quantitative reverse transcriptase PCR. Reporter gene expression is given as fold change (mean) relative to the wildtype yeast (white bar) and normalized to the reference gene 18S rRNA. Error bars represent standard error of the sample mean between triplicate samples. Statistics were calculated using two-tailed T-test between each individual sample and the wild type control: * P<0.05, ** P<0.01, *** P<0.001.

FIG. 7 shows a schematic representation of plasmid-based RNAi-effector expression construct.

FIG. 8 shows RNAi reporter expression profiles of integrated vs. plasmid-based RNAi-effector expression constructs. Total RNA from integrated and plasmid-based RNAi-effector expression constructs in both BY4742 wild type and BY4742 Δrrp6/Δski3 cells was used as input for quantitative reverse transcriptase PCR. Reporter gene expression is given as fold change (mean) relative to the wild type, genome integrated reporter yeast (BY4742 TRP1::blw) and normalized to the reference gene ALG9. Error bars represent standard error of the sample mean between triplicate samples. Statistics were calculated using two-tailed T-test between each individual sample and the reference sample described above: * P<0.05, ** P<0.01, *** P<0.001.

FIG. 9 shows a schematic representation of the RPR1 promoter-driven RNAi-effector expression construct. The RPR1 promoter-driven RNAi-effector expression construct was integrated into the yeast genome at the TRP1 locus.

FIG. 10 shows a schematic representation of the SNR33 promoter-driven RNAi-effector expression construct. The SNR33 promoter-driven RNAi-effector expression construct was maintained episomally on a 2-micron yeast plasmid, pRS343.

FIG. 11 shows RNAi reporter expression profiles of gene knockout strains bearing a genome-integrated RPR1 promoter-driven RNAi-effector expression construct. Total RNA from BY4742 wild type and BY4742 gene-knockout cells, all containing an RPR1 promoter-driven RNAi-effector expression construct integrated into the TRP1 locus, were used as input for quantitative reverse transcriptase PCR. Reporter gene expression is given as fold change (mean) relative to the wild type yeast (white bar) and normalized to the reference gene ALG9. Error bars represent standard error of the sample mean between triplicate samples. Statistics were calculated using two-tailed T-test between each individual sample and the reference sample described above: * P<0.05, ** P<0.01, *** P<0.001.

FIG. 12 shows RNAi reporter expression profiles of low and high copy plasmids bearing an SNR33 promoter-driven RNAi-effector expression construct. Total RNA from BY4742 wild type cells transformed with either low or high copy plasmids containing either a TEF1 promoter-driven or SNR33 promoter-driven RNAi-effector expression construct were used as input for quantitative reverse transcriptase PCR. Reporter gene expression is given as fold change (mean) relative to the wild type yeast and normalized to the reference gene ALG9. Error bars represent standard error of the sample mean between triplicate samples. Statistics were calculated using two-tailed T-test between each individual sample and the reference sample described above: * P<0.05, ** P<0.01, *** P<0.001.

FIG. 13 shows RNAi reporter expression profiles of different RNAi effector constructs. Total RNA from BY4742 wild type cells and BY4742 Δrrp6/Δski3 cells transformed with either genome-integrated or plasmid-based TEF1 promoter-driven RNAi-effector expression constructs were used as input for quantitative reverse transcriptase PCR. Reporter gene expression is given as fold change (mean) relative to the wild type yeast transformed with a single copy TRP1 locus integrated RNAi effector expression construct and normalized to the reference gene ALG9. Error bars represent standard error of the sample mean between triplicate samples. Statistics were calculated using two-tailed T-test between each individual sample and the reference sample described above: * P<0.05, ** P<0.01, *** P<0.001.

FIG. 14 shows survival of D. melanogaster adults during feeding trials with S. cerevisiae expressing hairpin RNA against bellwether (blw) (SEQ ID NO: 33). D. melanogaster adults were fed ad libitum with yeast expressing blw-dsRNA. The number of live adults in each vial was determined at each timepoint, and percentage survival values were calculated relative to the day 2 live flies. Values represent the means and standard deviations of 3 replicates vials, each containing 20 adult flies at time zero. Error bars represent 1 standard error of the mean.

FIG. 15 shows survival of Ae. aegypti larvae during feeding trials with S. cerevisiae hairpin RNA against fez2 (SEQ ID NO: 34). Survival of Aedes aegypti larvae fed on agar pellets containing yeast expressing fez2-dsRNA. The number of larvae after 24 h was determined, to correct for deaths due to handling injuries, and percentage survival values were calculated relative to the day 1 survivors. Values represent the means and standard errors of 4-6 replicates, starting with 40 larvae at time zero. Error bars represent 1 standard error of the mean.

FIG. 16A and FIG. 16B show LhRNA targeting IL-1β (SEQ ID NO: 41) reduced histological evidence of disease in SHIP deficient mice. 6-week-old SHIP deficient mice were treated with either yeast containing lhRNA or control yeast. Ileal cross-sections were fixed and stained with H&E and histological damage was scored in SHIP deficient mice after 10 days (FIG. 16A) or 14 days (FIG. 16B). N=4 mice per group in total.

FIGS. 17A, 17B and 17C show LhRNA targeting IL-1β (SEQ ID NO: 41) reduced disease activity and histological damage in DSS-treated Malt1^{-/-} mice. Malt1^{-/-} mice were subjected to 2% DSS for 6 days and were treated with either yeast containing lhRNA targeting IL-1β or control yeast. (FIG. 17A) Disease activity index was measured daily in mice during DSS treatment. (FIG. 17B) Colon cross-sections were fixed and stained with H&E and histological damage was scored. (FIG. 17C) Survival rate (>15% weight loss=humane end point) was calculated for Malt1^{-/-} mice. N=4 mice/group for one experiment.

FIG. 18 shows in vivo results for D. melanogaster embryos hatched and continually fed on dsRNA producing S. cerevisiae showing that the BY4742 Δski3 Δrrp6 mutant displays enhanced RNAi effector delivery compared to WT BY4742. Data shown are the eclosion rate and sum total of eclosed adults of D. melanogaster embryo's grown on BY4742 Δski3 Δrrp6 containing an empty vector plasmid, BY4742 WT yeast containing the plasmid-expressed long hairpin (lh) targeting eGFP, and BY4742 Δski3 Δrrp6 containing the same plasmid expressed lh eGFP.

### DETAILED DESCRIPTION

Unless otherwise indicated, the definitions and embodiments described in this and other sections are intended to be applicable to all embodiments and aspects of the present disclosure herein described for which they are suitable as would be understood by a person skilled in the art.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

As used herein, the singular forms “a”, “an” and “the” include plural references unless the content clearly dictates otherwise. The modifier “about” used in connection with a quantity is inclusive of the stated value and has the meaning dictated by the context (e.g., it includes the degree of error associated with measurement of the particular quantity). When referring to a period such as a year or annually, it includes a range from 9 months to 15 months. All ranges disclosed herein are inclusive of the endpoints, and the endpoints are independently combinable with each other.

The term “heterologous” as used herein refers to a sequence that is foreign to the host yeast cell.

The term “integrated” sequence as used herein refers to the foreign sequence being inserted into the host yeast genome.

### Yeast

The present disclosure provides a yeast cell comprising an RNA instability gene that is downregulated or inactivated and/or an RNA stability gene that is upregulated or heterologously expressed; and at least one heterologous sequence that encodes an RNA bioactive molecule. In an embodiment, the yeast cell comprises an RNA instability gene that is downregulated or inactivated or an RNA stability gene that is upregulated or heterologously expressed. In another embodiment, the yeast cell comprises an RNA instability gene that is downregulated or inactivated and an RNA stability gene that is upregulated or heterologously expressed.

In one embodiment, the at least one heterologous sequence is integrated into the yeast genome. In a particular embodiment, the at least one heterologous sequence is integrated at the trp locus. In another embodiment, the at least one heterologous sequence is present in the yeast in a plasmid.

In an embodiment, the at least one heterologous sequence comprises a constitutively active promoter for expressing the RNA bioactive molecule. In another embodiment, the at least one heterologous sequence comprises an inducible promoter for expressing the RNA bioactive molecule. In an embodiment, the at least one heterologous sequence comprises an RNA pol II promoter, such as an RNA pol II constitutively active promoter, for example TEF1. In another embodiment, the at least one heterologous sequence comprises an RNA pol III promoter, such as an RNA pol III constitutively active promoter, for example RPR1 or SNR33.

In an embodiment, the yeast is a food-grade yeast. In one embodiment, the yeast is from Saccharomyces. In a particular embodiment, the yeast is Saccharomyces cerevisiae.

In an embodiment, the yeast comprises at least one RNA stability gene that is upregulated or heterologously expressed and at least one RNA instability gene that is downregulated or inactivated.

The RNA stability gene may be any gene from any source that is involved in the production or stabilization of RNA in the yeast, such that upregulation or heterologous expression of said gene results in increased production or stabilization of RNA in the yeast, compared to a yeast where the RNA stability gene is not upregulated or heterologously expressed.

In an embodiment, the RNA stability gene that is upregulated or heterologously expressed comprises or consists of CCR4 or THP1. In another embodiment, the RNA stability gene that is upregulated or heterologously expressed comprises or consists of XRN1 or TAF1.

The term “CCR4” as used herein refers to CCR4 or Carbon Catabolite Repression 4 that may be from any yeast species or source, for example, S. cerevisiae or homologs thereof. S. cerevisiae CCR4 has the nucleic acid sequence as shown in Genbank Gene ID: 851212 or Saccharomyces Genome Database (SGD) No: S000000019 or NCBI Reference Sequence: NM_001178166.1. The term “THP1” as used herein refers to THP1 or Tho2/Hpr1 Phenotype that may be from any yeast species or source, for example, S. cerevisiae or homologs thereof. S. cerevisiae THP1 has the nucleic acid sequence as shown in Genbank Gene ID: 854082 or Saccharomyces Genome Database (SGD) No: S000005433 or NCBI Reference Sequence: NM_001183327.1.

The term “XRN1” as used herein refers to XRN1 or eXoRiboNuclease 1 that may be from any yeast species or source, for example, S. cerevisiae or homologs thereof. S. cerevisiae XRN1 has the nucleic acid sequence as shown in Genbank Gene ID: 852702 or Saccharomyces Genome Database (SGD) No: S000003141 or NCBI Reference Sequence: NM_001181038.1. The term “TAF1” as used herein refers to TAF1 or TATA binding protein-Associated Factor 1 that may be from any yeast species or source, for example, S. cerevisiae or homologs thereof. S. cerevisiae TAF1 has the nucleic acid sequence as shown in Genbank Gene ID: 853191 or Saccharomyces Genome Database (SGD) No: S000003506 or NCBI Reference Sequence: NM_001181403.2.

In an embodiment, the yeast comprises two RNA stability genes that are upregulated or heterologously expressed. In another embodiment, the yeast comprises three RNA stability genes that are upregulated or heterologously expressed. In a further embodiment, the yeast comprises four RNA stability genes that are upregulated or heterologously expressed. In yet a further embodiment, the yeast comprises 5, 6, 7, 8 or more RNA stability genes that are upregulated or heterologously expressed.

In an embodiment, the yeast comprises an expression cassette that is optionally integrated in its genome that codes for the RNA stability gene. In another embodiment, the yeast comprises a plasmid that codes for the RNA stability gene.

The yeast may be used to produce increased quantities of the RNA bioactive molecule or RNA bioactive molecules compared to a yeast where the RNA stability gene (or genes) has not been upregulated or heterologously expressed. In an embodiment, the production is increased by at least 1.25-fold, 2-fold, 5-fold, 10-fold, 50-fold, 100-fold, 500-fold, 1000-fold, 2000-fold or more.

The RNA instability gene may be any gene in the yeast that is involved in degradation or destabilization of RNA, such that downregulation or inactivation of said gene results in increased production or stabilization of RNA or decreased degradation of RNA, compared to a yeast where the RNA instability gene is not downregulated or inactivated.

In an embodiment, the RNA instability gene that is downregulated or inactivated comprises or consists of APN1, DBR1, DCS1, EDC3, HBS1, HTZ1, IPK1, LRP1, MAK10, MAK3, MAK31, MKT1, MPP6, MRT4, NAM7, NMD2, PAP2, POP2, RNH1, RNH203, RPS28A, RRP6, SIR3, SKI2, SKI3, SKI7, SKI8, SLH1, TRF8, or UPF3. In one embodiment, the RNA instability gene comprises or consists of HBS1, IPK1, LRP1, MAK10, MAK3, MAK31, MPP6, NAM7, NMD2, RRP6, SKI2, SKI3, or SKI7. In a particular embodiment, the RNA instability gene comprises or consists of LRP1. In another particular embodiment, the RNA instability gene comprises or consists of RRP6. In yet another particular embodiment, the RNA instability gene comprises or consists of SKI3. In a further particular embodiment, the RNA instability gene comprises or consists of MAK10. In yet a further particular embodiment, the RNA instability gene comprises or consists of MPP6.

The term “APN1” as used herein refers to APN1 or DNA-(apurinic or apyrimidinic site) lyase APN1 that may be from any yeast species or source, for example, S. cerevisiae or homologs thereof. S. cerevisiae APN1 has the nucleic acid sequence as shown in Genbank Gene ID: 853746 or Saccharomyces Genome Database (SGD) No: S000001597 or NCBI Reference Sequence: NM_001179680.1. The term “DBR1” as used herein refers to DBR1 or RNA lariat debranching enzyme that may be from any yeast source, for example, S. cerevisiae or homologs thereof. S. cerevisiae DBR1 has the nucleic acid sequence as shown in Genbank Gene ID: 853708 or SGD No. S000001632 or NCBI Reference Sequence: NM_001179715.1. The term “DCS1” as used herein refers to DCS1 or 5′-(N(7)-methyl 5′-triphosphoguanosine)-(mRNA) diphosphatase that may be from any yeast source, for example, S. cerevisiae or homologs thereof. S. cerevisiae DCS1 has the nucleic acid sequence as shown in Genbank Gene ID: 850974 or SGD No. S000004260 or NCBI Reference Sequence: NM_001182157.1. The term “EDC3” as used herein refers to EDC3 or Enhancer Of mRNA DeCapping that may be from any yeast source, for example S. cerevisiae or homologs thereof. S. cerevisiae EDC3 has the nucleic acid sequence as shown in Genbank Gene ID: 856700 or SGD No. S000000741 or NCBI Reference Sequence: NM_001178830.1. The term “HBS1” as used herein refers to HBS1 or ribosome dissociation factor GTPase HBS1 that may be from any yeast source, for example S. cerevisiae or homologs thereof. S. cerevisiae HBS1 has the nucleic acid sequence as shown in Genbank Gene ID: 853959 or SGD No. S000001792 or NCBI Reference Sequence: NM 001179874.3. The term “HTZ1” as used herein refers to HTZ1 or histone H2AZ that may be from any yeast source, for example S. cerevisiae or homologs thereof. S. cerevisiae HTZ1 has the nucleic acid sequence as shown in Genbank Gene ID: 854150 or SGD No. S000005372 or NCBI Reference Sequence: NM_001183266.1. The term “IPK1” as used herein refers to IPK1 or inositol pentakisphosphate 2-kinase that may be from any yeast source, for example S. cerevisiae or homologs thereof. S. cerevisiae IPK1 has the nucleic acid sequence as shown in Genbank Gene ID: 851910 or SGD No. S000002723 or NCBI Reference Sequence: NM_001180623.3. The term “LRP1” as used herein refers to LRP1 or Like RrP6 that may be from any yeast source, for example S. cerevisiae or homologs thereof. S. cerevisiae LRP1 has the nucleic acid sequence as shown in Genbank Gene ID: 856481 or SGD No. S000001123 or NCBI Reference Sequence: NM 001179211.1. The term “MAK3” as used herein refers to MAK3 or peptide alpha-N-acetyltransferase MAK3 that may be from any yeast source, for example S. cerevisiae or homologs thereof. S. cerevisiae MAK3 has the nucleic acid sequence as shown in Genbank Gene ID: 856163 or SGD No. S000006255 or NCBI Reference Sequence: NM_001184148.1. The term “MAK10” as used herein refers to MAK10 or Maintenance of Killer 10 that may be from any yeast source, for example S. cerevisiae or homologs thereof. S. cerevisiae MAK10 has the nucleic acid sequence as shown in Genbank Gene ID: 856657 or SGD No. S000000779 or NCBI Reference Sequence: NM_001178868.3. The term “MAK31” as used herein refers to MAK31 or Maintenance of Killer 31 that may be from any yeast source, for example S. cerevisiae or homologs thereof. S. cerevisiae MAK31 has the nucleic acid sequence as shown in Genbank Gene ID: 850383 or SGD No. S000000614 or NCBI Reference Sequence: NM_001178734.1. The term “MKT1” as used herein refers to MKT1 or Maintenance of K2 Killer Toxin that may be from any yeast source, for example S. cerevisiae or homologs thereof. S. cerevisiae MKT1 has the nucleic acid sequence as shown in Genbank Gene ID: 855639 or SGD No. S000005029 or NCBI Reference Sequence: NM_001182923.3. The term “MPP6” as used herein refers to MPP6 or M-Phase Phosphoprotein 6 homolog that may be from any yeast source, for example S. cerevisiae or homologs thereof. S. cerevisiae MPP6 has the nucleic acid sequence as shown in Genbank Gene ID: 855758 or SGD No. S000005307 or NCBI Reference Sequence: NM_001183201.3. The term “MRT4” as used herein refers to MRT4 or mRNA Turnover 4 that may be from any yeast source, for example S. cerevisiae or homologs thereof. S. cerevisiae MRT4 has the nucleic acid sequence as shown in Genbank Gene ID: 853860 or SGD No. S000001492 or NCBI Reference Sequence: NM_001179575.1. The term “NAM7” as used herein refers to NAM7 or ATP-dependent RNA helicase NAM7 that may be from any yeast source, for example S. cerevisiae or homologs thereof. S. cerevisiae NAM7 has the nucleic acid sequence as shown in Genbank Gene ID: 855104 or SGD No. S000004685 or NCBI Reference Sequence: NM_001182579.1. The term “NMD2” as used herein refers to NMD2 or Nonsense-mediated MRNA Decay may be from any yeast source, for example S. cerevisiae or homologs thereof. S. cerevisiae NMD2 has the nucleic acid sequence as shown in Genbank Gene ID: 856476 or SGD No. S000001119 or NCBI Reference Sequence: NM_001179207.1. The term “PAP2” as used herein refers to PAP2 or non-canonical poly(A) polymerase PAP2 that may be from any yeast source, for example S. cerevisiae or homologs thereof. S. cerevisiae PAP2 has the nucleic acid sequence as shown in Genbank Gene ID: 854034 or SGD No. S000005475 or NCBI Reference Sequence: NM_001183369.1. The term “POP2” as used herein refers to POP2 or CCR4-NOT core DEDD family RNase subunit POP2 that may be from any yeast source, for example S. cerevisiae or homologs thereof. S. cerevisiae POP2 has the nucleic acid sequence as shown in Genbank Gene ID: 855788 or SGD No. S000005335 or NCBI Reference Sequence: NM_001183229.3. The term “RNH1” as used herein refers to RNH1 or RNA-DNA hybrid ribonuclease that may be from any yeast source, for example S. cerevisiae or homologs thereof. S. cerevisiae RNH1 has the nucleic acid sequence as shown in Genbank Gene ID: 855274 or SGD No. S000004847 or NCBI Reference Sequence: NM_001182741.1. The term “RNH203” as used herein refers to RNH203 or Rnh203p that may be from any yeast source, for example S. cerevisiae or homologs thereof. S. cerevisiae RNH203 has the nucleic acid sequence as shown in Genbank Gene ID: 850847 or SGD No. S000004144 or NCBI Reference Sequence: NM 001182041.1. The term “RPS28A” as used herein refers to RPS28A or ribosomal 40S subunit protein S28A that may be from any yeast source, for example S. cerevisiae or homologs thereof. S. cerevisiae RPS28A has the nucleic acid sequence as shown in Genbank Gene ID: 854338 or SGD No. S000005693 or NCBI Reference Sequence: NM_001183586.1. The term “RRP6” as used herein refers to RRP6 or exosome nuclease subunit RRP6 that may be from any yeast source, for example S. cerevisiae or homologs thereof. S. cerevisiae RRP6 has the nucleic acid sequence as shown in Genbank Gene ID: 854162 or SGD No. S000005527 or NCBI Reference Sequence: NM_001183420.1. The term “SIR3” as used herein refers to SIR3 or chromatin-silencing protein SIR3 that may be from any yeast source, for example S. cerevisiae or homologs thereof. S. cerevisiae SIR3 has the nucleic acid sequence as shown in Genbank Gene ID: 851163 or SGD No. S000004434 or NCBI Reference Sequence: NM_001182330.3. The term “SKI2” as used herein refers to SKI2 or SKI complex RNA helicase subunit SKI2 that may be from any yeast source, for example S. cerevisiae or homologs thereof. S. cerevisiae SKI2 has the nucleic acid sequence as shown in Genbank Gene ID: 851114 or SGD No. S000004390 or NCBI Reference Sequence: NM_001182286.3. The term “SKI3” as used herein refers to SKI3 or SKI complex subunit tetratricopeptide repeat protein SKI3 that may be from any yeast source, for example S. cerevisiae or homologs thereof. S. cerevisiae SKI3 has the nucleic acid sequence as shown in Genbank Gene ID: 856319 or SGD No. S000006393 or NCBI Reference Sequence: NM_001184286.1. The term “SKI7” as used herein refers to SKI7 or Superkiller 7 that may be from any yeast source, for example S. cerevisiae or homologs thereof. S. cerevisiae SKI7 has the nucleic acid sequence as shown in Genbank Gene ID: 854243 or SGD No. S000005602 or NCBI Reference Sequence: NM_001183495.1. The term “SKI8” as used herein refers to SKI8 or SKI complex subunit WD repeat protein SKI8 that may be from any yeast source, for example S. cerevisiae or homologs thereof. S. cerevisiae SKI8 has the nucleic acid sequence as shown in Genbank Gene ID: 852659 or SGD No. S000003181 or NCBI Reference Sequence: NM_001181078.1. The term “SLH1” as used herein refers to SLH1 or putative RNA helicase that may be from any yeast source, for example S. cerevisiae or homologs thereof. S. cerevisiae SLH1 has the nucleic acid sequence as shown in Genbank Gene ID: 853187 or SGD No. S000003503 or NCBI Reference Sequence: NM_001181400.4. The term “TRF5” as used herein refers to TRF5 or non-canonical poly(A) polymerase TRF5 that may be from any yeast source, for example S. cerevisiae or homologs thereof. S. cerevisiae TRF5 has the nucleic acid sequence as shown in Genbank Gene ID: 855417 or SGD No. S000005243 or NCBI Reference Sequence: NM 001183137.1. The term “UPF3” as used herein refers to UPF3 or UP Frameshift that may be from any yeast source, for example S. cerevisiae or homologs thereof. S. cerevisiae UPF3 has the nucleic acid sequence as shown in Genbank Gene ID: 852963 or SGD No. S000003304 or NCBI Reference Sequence: NM_001181201.1.

The term “homolog” as used herein refers to the same gene in a related species such as the same gene in a different yeast strain. Typically homologs share a high degree of sequence identity, such as at least 50%, 60%, 70% or more. The homology between two genes that are derived from species which are more closely related is typically higher than from more distantly related species.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. An optional, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present disclosure. BLAST protein searches can be performed with the XBLAST program parameters set, e.g., to score-50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search, which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another optional, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In an embodiment, the yeast comprises two RNA instability genes that are downregulated or inactivated.

In one embodiment, the RNA instability genes that are downregulated or inactivated in the yeast comprise or consist of RRP6 and SKI3. In another embodiment, the RNA instability genes that are downregulated or inactivated in the yeast comprise or consist of LRP1 and RRP6. In yet another embodiment, the RNA instability genes that are downregulated or inactivated in the yeast comprise or consist of LRP1 and MAK3. In a further embodiment, the RNA instability genes that are downregulated or inactivated in the yeast comprise or consist of LRP1 and SKI2. In yet a further embodiment, the RNA instability genes that are downregulated or inactivated in the yeast comprise or consist of SKI2 and SKI3. In an even further embodiment, the RNA instability genes that are downregulated or inactivated in the yeast comprise or consist of SKI3 and MAK3.

In yet another embodiment, the yeast comprises three RNA instability genes that are downregulated or inactivated. In a further embodiment, the yeast comprises four RNA instability genes that are downregulated or inactivated. In yet a further embodiment, the yeast comprises 5, 6, 7, 8 or more RNA instability genes that are downregulated or inactivated.

In an embodiment, the yeast that comprises the RNA instability gene that is downregulated or inactivated comprises a genome where the RNA instability gene has been deleted. In another embodiment, the yeast that comprises the RNA instability gene that is downregulated or inactivated comprises a genome where the RNA instability gene is downregulated or inactivated by any modification that reduces or abolishes its function, such as by truncation, introduction of a stop codon or by point mutation. In yet another embodiment, the yeast may heterologously express factors that degrade or otherwise inactivate the protein product of the RNA instability gene (e.g. a dominant negative allele).

The yeast may be used to produce increased quantities of the RNA bioactive molecule or RNA bioactive molecules compared to a yeast where the RNA instability gene (or genes) has not been downregulated or inactivated. In an embodiment, the production is increased by at least 1.25-fold, 2-fold, 5-fold, 10-fold, 50-fold, 100-fold, 500-fold, 1000-fold, 2000-fold or more

The RNA bioactive molecule refers to any biologically active RNA molecule, from any source or organism. In one embodiment, the RNA bioactive molecule is an mRNA molecule for producing a protein. In another embodiment, the RNA bioactive molecule is an RNAi effector molecule for inducing an RNA interference response.

In an embodiment, the mRNA bioactive molecule encodes a protein that is useful for the treatment of a disease and/or infection, optionally immune factors that negatively regulate infection, such as stimulatory cytokines for macrophages; a protein that is related to a protein deficiency; or a protein that can elicit an immune response for prevention or treatment of disease and/or infection. Examples of mRNA that would be useful for therapy are known in the art, including without limitation, vascular endothelial growth factor (VEGF) and cystic fibrosis transmembrane conductance regulator (CFTR) (Trepotec et al. 2018), ornithine transcarbamylase (Prieve et al. 2018), glucose-6-phosphate (Roseman et al. 2018), and Influenza hemagluttinins, Ebola virus glycoprotein, RSV-F, Rabies virus glycoprotein, HIV-1 gag, HSV1-tk, hMUT, hEPO, Bcl-2 and ACE-2 (Xiong et al. 2018), and SERPINA1 (Connolly et al. 2018).

In an embodiment, the RNAi effector molecule is siRNA, miRNA, lhRNA, shRNA, dsRNA, or anti-sense RNA. In one embodiment, the RNAi effector molecule is dsRNA. In another embodiment, the RNAi effector molecule is long hairpin RNA (lhRNA).

The terms “RNA interference,” “interfering RNA” or “RNAi” refer to single-stranded RNA or double-stranded RNA (dsRNA) that is capable of reducing or inhibiting expression of a target nucleic acid by mediating the degradation of mRNAs which are complementary to the sequence of the interfering RNA when the interfering RNA is in the same cell as the target gene. Interfering RNA may have substantial or complete identity to the target nucleic acid or may comprise a region of mismatch.

The term “antisense RNA” refers to a single stranded RNA that is complementary to messenger RNA and that hybridizes with the messenger RNA blocking translation into protein.

The term “long hairpin RNA” or “lhRNA” as used herein refers to a long inhibitor RNA that can be used to reduce or inhibit expression of a target nucleic acid by RNA interference. LhRNA are typically single stranded with secondary structure (hairpin) and longer than 60 nucleotides. Total length may be 1000 base pairs or more.

The term “siRNA” or “siRNA oligonucleotide” refers to a short inhibitory RNA that can be used to reduce or inhibit nucleic acid expression of a specific nucleic acid by RNA interference.

The siRNA can be a duplex, a short RNA hairpin (shRNA) or a microRNA (miRNA).

Methods of designing specific nucleic acid molecules that silence gene expression and administering them are known to a person skilled in the art. For example, it is known in the art that efficient silencing is obtained with siRNA duplex complexes paired to have a two nucleotide 3′ overhang. The siRNA can also be chemically modified to increase stability. For example adding two thymidine nucleotides and/or 2′O methylation is thought to add nuclease resistance. Other modifications include the addition of a 2′-O-methyoxyethyl, 2′-O-benzyl, 2′-O-methyl-4-pyridine, C-allyl, 0-allyl, O-alkyl, O-alkylthioalkyl, O-alkoxylalkyl, alkyl, alkylhalo, O-alkylhalo, F, NH2, ONH2, O-silylalkyl, or N-phthaloyl group (see U.S. Pat. No. 7,205,399; Kenski et al. Mol. Ther. Nucl. Acids 1:1-8 (2012); Behlke, Oligonucleotides 18:305-320 (2008)). Other modifications include direct modification of the internucleotide phosphate linkage, for example replacement of a non-bridging oxygen with sulfur, boron (boranophosphate), nitrogen (phosphoramidate) or methyl (methylphosphonate). A person skilled in the art will recognize that other nucleotides can also be added and other modifications can be made. As another example deoxynucleotide residues (e.g. dT) can be employed at the 3′ overhang position to increase stability.

The RNAi effector molecule may be any RNAi effector molecule that targets a gene of interest. In an embodiment, the gene of interest is involved in survival, maturation, reproduction, aggressiveness, or virulence of an unwanted organism, such as a pest, parasite, bacterium, fungus or virus. In another embodiment, the gene of interest is involved in promoting a disease state in an organism.

In an embodiment, the yeast comprises at least two heterologous sequences that encode an RNA bioactive molecule, such that two different RNA bioactive molecules are produced. In another embodiment, the yeast comprises at least three, at least four, at least five or more heterologous sequences that encode an RNA bioactive molecule, such that different RNA bioactive molecules are produced in the yeast.

### Methods

The present disclosure also provides a method of making a yeast cell that produces an increased amount of RNA bioactive molecules, the method comprising downregulating or inactivating an RNA instability gene(s) as disclosed herein or upregulating and/or heterologously expressing an RNA stability gene(s) as disclosed herein in the yeast; and expressing at least one heterologous sequence that encodes the RNA bioactive molecule. In an embodiment, the method comprises downregulating or inactivating an RNA instability gene as disclosed herein or upregulating or heterologously expressing an RNA stability gene as disclosed herein in the yeast. In another embodiment, the method comprises downregulating or inactivating an RNA instability gene as disclosed herein and upregulating or heterologously expressing an RNA stability gene as disclosed herein in the yeast. In an embodiment, at least one RNA stability gene is upregulated or heterologously expressed and at least one RNA instability gene is downregulated or inactivated.

In an embodiment, the method comprises integrating the heterologous sequence into the yeast genome, for example, at the trp locus. In another embodiment, the method comprises inserting a plasmid into the yeast that codes for the heterologous sequence.

In an embodiment, the RNA stability gene that is upregulated or heterologously expressed comprises or consists of CCR4 or THP1. In another embodiment, the RNA stability gene that is upregulated or heterologously expressed comprises or consists of XRN1 or TAF1.

In an embodiment, the RNA instability gene that is downregulated or inactivated comprises or consists of APN1, DBR1, DCS1, EDC3, HBS1, HTZ1, IPK1, LRP1, MAK10, MAK3, MAK31, MKT1, MPP6, MRT4, NAM7, NMD2, PAP2, POP2, RNH1, RNH203, RPS28A, RRP6, SIR3, SKI2, SKI3, SKI7, SKIS, SLH1, TRF5, or UPF3. In one embodiment, the RNA instability gene comprises or consists of HBS1, IPK1, LRP1, MAK10, MAK3, MAK31, MPP6, NAM7, NMD2, RRP6, SKI2, SKI3, or SKI7. In a particular embodiment, the RNA instability gene comprises or consists of LRP1. In another particular embodiment, the RNA instability gene comprises or consists of RRP6. In yet another particular embodiment, the RNA instability gene comprises or consists of SKI3. In a further particular embodiment, the RNA instability gene comprises or consists of MAK10. In yet a further particular embodiment, the RNA instability gene comprises or consists of MPP6.

In an embodiment, the method comprises downregulating or inactivating two RNA instability genes and/or upregulating or heterologously expressing two RNA stability genes.

In one embodiment, the two RNA instability genes comprise or consist of RRP6 and SKI3. In another embodiment, the two RNA instability genes comprise or consist of LRP1 and RRP6. In yet another embodiment, the two RNA instability genes comprise or consist of LRP1 and MAK3. In a further embodiment, the two RNA instability genes comprise or consist of LRP1 and SKI2. In yet a further embodiment, the two RNA instability genes comprise or consist of SKI2 and SKI3. In an even further embodiment, the two RNA instability genes comprise or consist of SKI3 and MAK3.

In yet another embodiment, the method comprises downregulating or inactivating three RNA instability genes and/or upregulating or heterologously expressing three RNA stability genes. In a further embodiment, the method comprises downregulating or inactivating four RNA instability genes and/or upregulating or heterologously expressing four RNA stability genes. In yet a further embodiment, the method comprises downregulating or inactivating 5, 6, 7, 8 or more RNA instability genes and/or upregulating or heterologously expressing 5, 6, 7, 8 or more RNA stability genes.

In an embodiment, downregulating or inactivating the RNA instability gene comprises deleting the RNA instability gene or otherwise modifying the yeast to reduce or abolish its function, for example, by truncation, introduction of a stop codon or by point mutation. A person skilled in the art would readily understand how to make a deletion of a yeast gene. Briefly, gene deletions can be obtained by any mutation, or combination thereof, that result in the partial or complete loss of protein function. For example, suitable mutations may include, but are not limited to, loss of promoter activity, loss of RNA translation, protein truncation, amino acid substitution, loss of coding sequence, etc. Such mutations can be achieved through multiple means of genome modification including, but not limited to, replacing all or a portion of a gene with target DNA encoding the desired mutation via homologous recombination, CRISPR/Cas9 genome editing, or other forms of gene editing.

In an embodiment, heterologously expressing the RNA stability gene or genes comprises integrating an expression cassette comprising the heterologous RNA stability gene or genes into the yeast genome. Briefly, such a cassette would comprise the RNA stability gene operationally linked to promoter and terminator sequences suitable for driving expression of the RNA stability gene. Such promoter and terminator sequences are generally known to those skilled in the art and include, but are not limited to, TEF1, PGK1, TDH3, REV1, RNR2, GAL1, ADH1, etc. The cassette may be integrated into the yeast genome through multiple means of genome modification including, but not limited to, homologous recombination, CRISPR/Cas9 genome editing, or other forms of gene editing. In another embodiment, heterologously expressing the RNA stability gene or genes comprises the use of a plasmid to drive expression of the gene expression cassette(s).

Accordingly, in an embodiment, the at least one heterologous sequence comprises a constitutively active promoter for expressing the RNA bioactive molecule. In another embodiment, the at least one heterologous sequence comprises an inducible promoter for expressing the RNA bioactive molecule. In an embodiment, the at least one heterologous sequence comprises an RNA pol II promoter such as an RNA pol II constitutively active promoter, for example TEF1. In another embodiment, the at least one heterologous sequence comprises an RNA pol III promoter, such as an RNA pol III constitutively active promoter, for example RPR1 or SNR33.

In yet another embodiment, the yeast may heterologously express factors that degrade or otherwise inactivate the protein product of the RNA instability gene (e.g. a dominant negative allele).

The yeast cells disclosed herein are useful as a continuous source or delivery system of RNA bioactive molecules for a variety of applications.

For example, RNA interference molecules have been shown to be an effective biocontrol agent. For example, bacteria have been used to deliver dsRNA to control insects (Zhu et al., 2010; Whitten et al., 2016) and insect vectors of disease (Taracena et al., 2015). The potential of yeast to be used as a biocontrol agent for insects has also been shown in a number of recent publications. For example, common S. cerevisiae expressing shRNA targeting Drosophila suzukii—a major cause of crop loss in soft summer fruit including cherries, blueberries, grapes and apricots—was shown to reduce activity and reproductive fitness (Murphy et al. 2016, WO2017106171A1). More recently, common S. cerevisiae was also engineered as hosts for shRNA expression targeting various genes required for viability of mosquito larvae (Hapairai, Mysore, Chen, & Harper, 2017; Mysore, Hapairai, & Sun, 2017). In all cases common, unoptimized yeast were used, thus the systems were not optimized for dsRNA production and/or delivery. It follows that having a biological delivery system that produces increased amount of RNA may be useful for biocontrol.

Accordingly, herein provided is a method of biocontrol comprising exposing an unwanted organism to a yeast cell that produces increased amounts of a RNA bioactive molecule, such as mRNA encoding a toxic factor or a negative regulatory factor, or an RNAi effector molecule(s) as disclosed herein, wherein the bioactive molecule reduces the survival, maturation, reproduction, aggressiveness, or virulence of the unwanted organism, for example, an RNAi effector molecule(s) targets a gene in the unwanted organism that is responsible for survival, maturation, reproduction, aggressiveness, or virulence. In an embodiment, the unwanted organism is a pest, a bacterium, a virus, a fungus or a parasite.

In an embodiment, exposing the unwanted organism to the yeast cell comprises feeding the yeast cell to the unwanted organism, or feeding the yeast cell to a host organism harboring the unwanted organism (e.g. host organism infected with a bacterium, virus, fungus or parasite).

In one embodiment, the unwanted organism is an agricultural pest, such as an insect, and the RNAi effector molecule(s) targets and silences the expression of at least one gene required by the pest for survival, maturation, reproduction, aggressiveness, or virulence. For example, RNAi effector molecules have been known to target survival genes such as actin, VATPase and cytochrome P450 (Anderson, Sheehan, Eckholm, & Mott, 2011; Chang, Wang, Regev-Yochay, Lipsitch, & Hanage, 2014; Jin, Singh, Li, & Zhang, 2015; X. Li, Zhang, & Zhang, 2011; Lin, Huang, Liu, & Belles, 2017; Murphy, Tabuloc, Cervantes, & Chiu, 2016), maturation genes such as hemolin and hunchback (Yu, Liu, Huang, & Chen, 2016) and reproduction genes such as vitellogenin (Vg) (Ghosh, Hunter, & Park, 2017; Lu, Vinson, & Pietrantonio, 2009; Whitten, Facey, & Del Sol, 2016).

Accordingly, in one embodiment, the pest is a fly and the gene required by the pest for survival is bellwether (blw). Bellwether encodes a subunit of the mitochondrial ATP synthase complex involved in the final enzymatic step of the oxidative phosphorylation pathway (Jacobs et al. 1998). Moreover, bellwether expression is known to regulate Drosophila lifespan in male flies (Garcia et al. 2017).

As shown in Example 9, targeting the gene neurexin IV in flies results in reduced survival. Accordingly, in another embodiment, the pest is a fly and the gene required by the pest for survival is neurexin IV (NrxIV).

In another embodiment, the pest is a mosquito and the gene required by the pest for survival is fez2. Fez2 encodes fasciculation and elongation protein zeta 2 (fez2), which is an essential neuronal factor necessary for normal axonal bundling and elongation within axon bundles (Fujita et al. 2004). Moreover, fez2 knockdown has been shown to significantly decrease viability of mosquito larvae (Hapairai et al. 2017).

mRNA molecules and RNA interference molecules also have applications in the treatment of disease. Accordingly, also provided herein is a method of treating a disease comprising exposing a subject having the disease to a yeast cell that produces increased amounts of an RNA bioactive molecule(s) as disclosed herein, wherein the RNA bioactive molecule(s) is useful for treating the disease. Also provided herein is use of a yeast cell that produces increased amounts of an RNA bioactive molecule(s) as disclosed herein for treating a disease in a subject, wherein the RNA bioactive molecule(s) is useful for treating the disease. Further provided herein is use of a yeast cell that produces increased amounts of an RNA bioactive molecule(s) as disclosed herein in the preparation of a medicament for treating a disease in a subject, wherein the RNA bioactive molecule(s) is useful for treating the disease. Even further provided is a yeast cell that produces increased amounts of an RNA bioactive molecule(s) as disclosed herein for use in treating a disease in a subject, wherein the RNA bioactive molecule(s) is useful for treating the disease.

In an embodiment, the organism is an aquaculture species, livestock, a companion animal, a plant or a human or any other animal. In the case of livestock/aquaculture species and humans or any other animal, the yeast cell containing RNA bioactive molecules can be fed to the organism in either a live or inactivated form. Other routes of administration or use include intravenous, intradermal, intramuscular and subcutaneous injections as well as topical use or spraying of a solution containing the yeast.

In one embodiment, the RNA bioactive molecule is an mRNA that encodes a protein that is useful for the treatment of the disease, an mRNA that encodes a protein that is related to a protein deficiency or an mRNA that encodes a protein that can elicit an immune response for prevention or treatment of the disease.

In an embodiment, the mRNA bioactive molecule encodes a protein that is useful for the treatment of a disease and/or infection, a protein that is related to a protein deficiency or a protein that can elicit an immune response for prevention or treatment of disease and/or infection. Examples of mRNA that would be useful for therapy are known in the art, including without limitation, vascular endothelial growth factor (VEGF) and cystic fibrosis transmembrane conductance regulator (CFTR) (Trepotec et al. 2018), ornithine transcarbamylase (Prieve et al. 2018), glucose-6-phosphate (Roseman et al. 2018), and Influenza hemagluttinins, Ebola virus glycoprotein, RSV-F, Rabies virus glycoprotein, HIV-1 gag, HSV1-tk, hMUT, hEPO, Bcl-2 and ACE-2 (Xiong et al. 2018), and SERPINA1 (Connolly et al. 2018).

In another embodiment, the RNA bioactive molecule is an RNAi effector molecule that targets a disease promoting gene in the subject. In an embodiment, the disease is a disease affecting the gut of the subject. In an embodiment, exposing the subject to the yeast comprises feeding the yeast cells to the subject.

Once delivered to an organism, RNAi typically enters the organism's cells via endosomes, RNAi effectors are released into the cell, and then proceed to downregulate target disease genes through commonly accepted functional RISC RNA-protein complexes readily known to those skilled in the art, thereby eliminating or protecting the organism from diseases or pests (Bradford et al., 2017).

In an embodiment, the disease (e.g. bacterial, viral, fungal or other parasite) promoting gene that is targeted is selected from one of the following classes including, but not limited to, native disease genes required for replication and/or survival, native disease genes required for virulence, host genes required for disease state (e.g. host factors responsible for infection), or host genes preventing immune system clearance of the disease (e.g. host factors attenuating immune response to the disease), and host genes that promote disease state.

Actin, VATPase and cytochrome P450 have been shown to be genes involved in survival (Anderson et al., 2011; Chang et al., 2014; Jin et al., 2015; Li et al. 2011; Lin et al., 2017; Murphy et al., 2016). Accordingly, in one embodiment, the disease promoting gene is actin, VATPase or cytochrome p450.

Hemolin and hunchback have been shown to be genes involved in maturation (Yu, Liu, Huang, & Chen, 2016). Accordingly, in another embodiment, the disease promoting gene is hemolin or hunchback.

Vitellogenin has been shown to be a gene involved in reproduction (Ghosh et al., 2017; Lu et al., 2009; Whitten et al., 2016). Accordingly, in another embodiment, the disease promoting gene is vitellogenin.

VEGF, VEGFR1, and DDIT4 have been shown to play a role in age-related macular degeneration (Tiemann & Rossi, 2009). Accordingly, in an embodiment, the disease promoting gene is VEGF, VEGFR1, or DDIT4.

KRT6A has been shown to play a role in pachyonychia congenita (Tiemann & Rossi, 2009). Accordingly, in another embodiment, the disease promoting gene is KRT6A.

RRM2 has been shown to play a role in solid tumour formation (Tiemann & Rossi, 2009). Accordingly, in another embodiment, the disease promoting gene is RRM2.

p53 has been shown to play a role in acute renal failure (Tiemann & Rossi, 2009). Accordingly, in another embodiment, the disease promoting gene is p53.

LMP2, LMP7, and MECL1 have been shown to play a role in metastatic melanoma (Tiemann & Rossi, 2009). Accordingly, in another embodiment, the disease promoting gene is LMP2, LMP7, or MECL1.

TNF-α has been shown to play a role in colon inflammation (Laroui et al., 2011). Accordingly, in another embodiment, the disease promoting gene is TNF-α.

IL-1β is a pro-inflammatory cytokine, which is known to be a primary regulator of inflammation (Coccia et al. 2012). IL-1β plays a key role in the development of IBD by activating multiple types of immune cells. Progression of intestinal inflammation in patients with IBD is associated with increased levels of IL-1β production (Coccia et al. 2012). Accordingly, in another embodiment, the disease promoting gene is IL-1β, for example, for treating IBD.

RNA bioactive molecules also have applications in fighting infection. Interfering RNA can target genes within an infectious organism in order to decrease the infectivity or survival of the infectious organism and mRNA molecules can encode proteins that are useful in treating the infection or proteins that elicit an immune response against the infection.

Accordingly, also provided herein is a method of treating or preventing an infection in a subject comprising exposing a subject having the infection, or susceptible to the infection, to a yeast cell that produces increased amount of an RNA bioactive molecule(s) as disclosed herein, wherein the RNA bioactive molecule(s) is useful for treating or preventing the infection. Further provided is use of a yeast cell that produces increased amount of an RNA bioactive molecule(s) as disclosed herein for treating or preventing an infection in a subject, wherein the RNA bioactive molecule(s) is useful for treating or preventing the infection. Even further provided is use of a yeast cell that produces increased amount of an RNAi effector molecule(s) as disclosed herein in the preparation of a medicament for treating or preventing an infection in a subject, wherein the RNA bioactive molecule(s) is useful for treating or preventing the infection. Also provided is use of a yeast cell that produces increased amount of an RNA bioactive molecule(s) as disclosed herein for use in treating or preventing an infection in a subject, wherein the RNA bioactive molecule(s) is useful for treating or preventing the infection.

In an embodiment, the yeast cell is exposed to the subject or used orally.

In an embodiment, the organism causing the infection is a virus, fungus, parasite or bacterium.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Reporter Strain Development

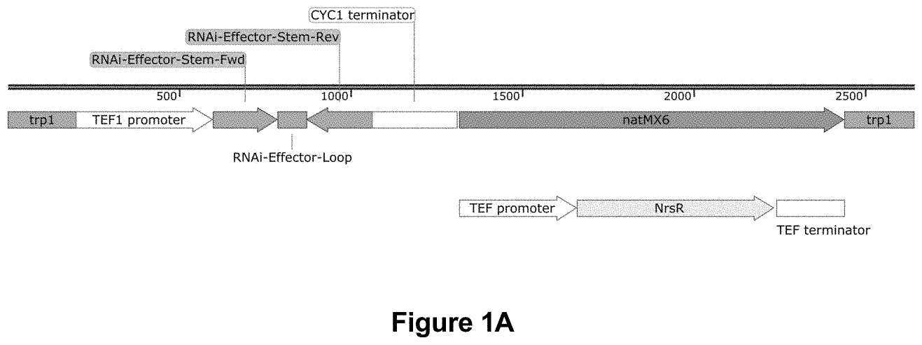

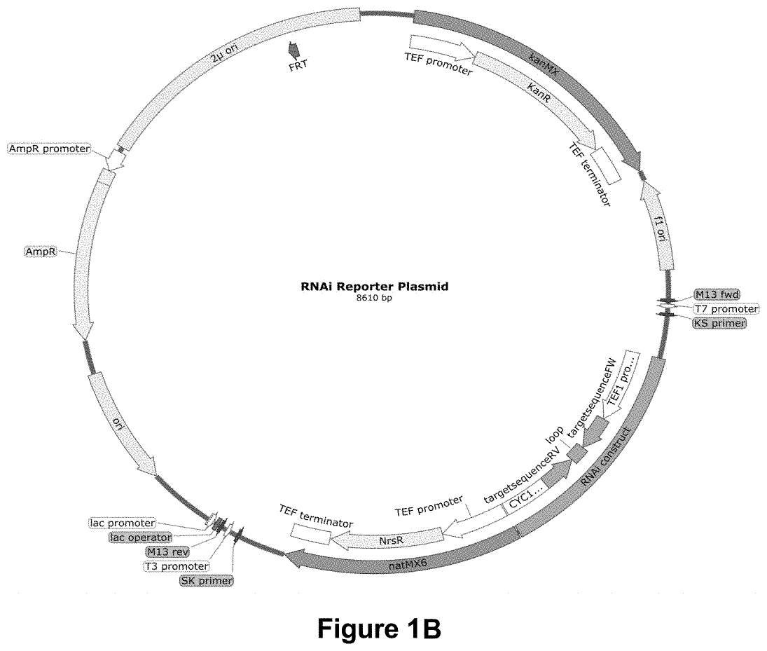

To be able to assess the impact of yeast modifications on RNAi effector expression, a reporter RNAi reporter gene system was developed containing the constitutive strong promoter (TEF1), short hairpin DNA sequence targeting D. suzukii tubulin (˜200 bp stem sequences and 74 bp loop sequence), CYC1 terminator, NatMX resistance marker cassette and TRP1 flanking regions. This cassette was assembled in expression vector pRS423-KanMX using Gibson cloning (SEQ ID NO:1). All the fragments required for the Gibson reaction were PCR amplified (SEQ ID 3, 4, 5, 6, 7, 8) and purified except the backbone vector (digested by EcoRV) and reporter gene cassette (digested by KpnI and SaII). The assembled 2.6 kb reporter system (SEQ ID NO:2) was harvested by restriction enzyme digestion (Bst1107Z) followed by DNA gel purification. A schematic diagram of this construct is shown in FIG. 1. The purified DNA fragment was used as donor DNA for integrating the reporter system into the TRP1 locus of the haploid laboratory S. cerevisiae strain Y7092 (MAT alpha, can1delta::STE2pr-Sp_his5 lyp1delta his3delta1 leu2de1ta0 ura3delta0 met15delta0) using CRISPR/Cas9 technology (DiCarlo, Norville, Mali, & Rios, 2013) or homologous recombination (SEQ ID NO: 9 and 10).

The RNAi reporter construct was integrated into the genome of a haploid S. cerevisiae laboratory strain (Y7092), after which the reporter-containing query strain was crossed to the Stanford Yeast Deletion Genome Project collection (Winzeler, Shoemaker, & Astromoff, 1999). Following re-isolation of stable haploid strains, a set of 5000+ yeast deletion mutants were obtained, each of which contained a singular copy of the RNAi reporter constructs.

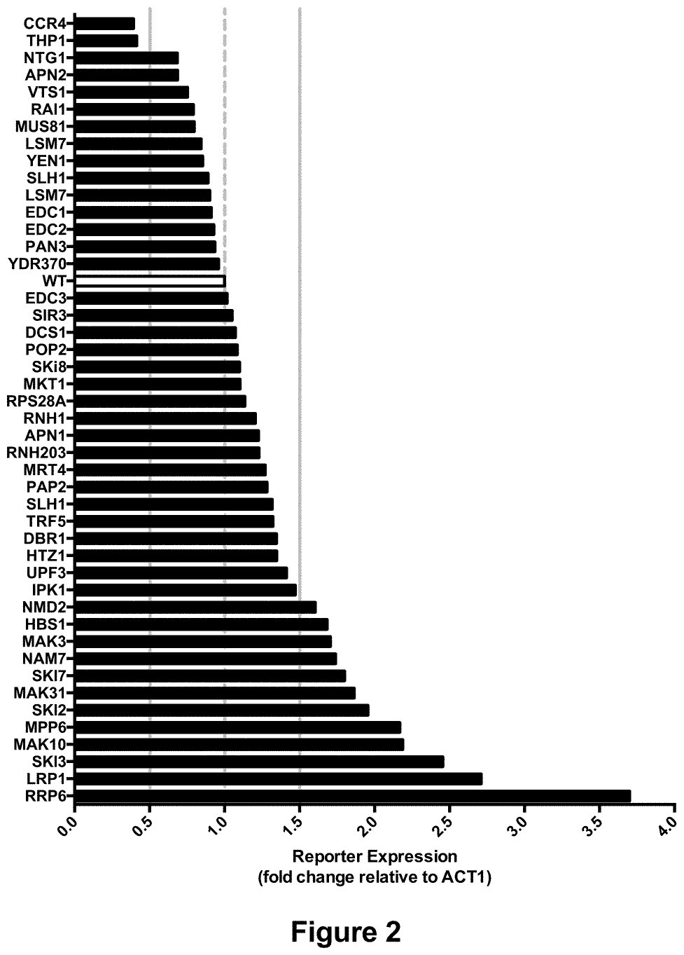

Using the research literature on yeast RNA processing as a guideline, a set of 350+ gene knockouts were shortlisted to test for their ability to increase steady state levels of the RNAi reporter. From this list, the RNAi reporter expression, via RT-qPCR, was assayed. In brief, total RNA was isolated using the hot acidic phenol-chloroform extraction protocol (Köhrer & Domdey, 1991), purified, and reverse transcribed into cDNA. Quantitative PCR was performed using either ACT1 or ALG1 as housekeeping genes (SEQ ID NO: 28, 29, 30, 31, 71, 72). The 46 most interesting knockouts derived from the short list of gene ontology and genetic interaction information were screened for reporter expression (FIG. 2).

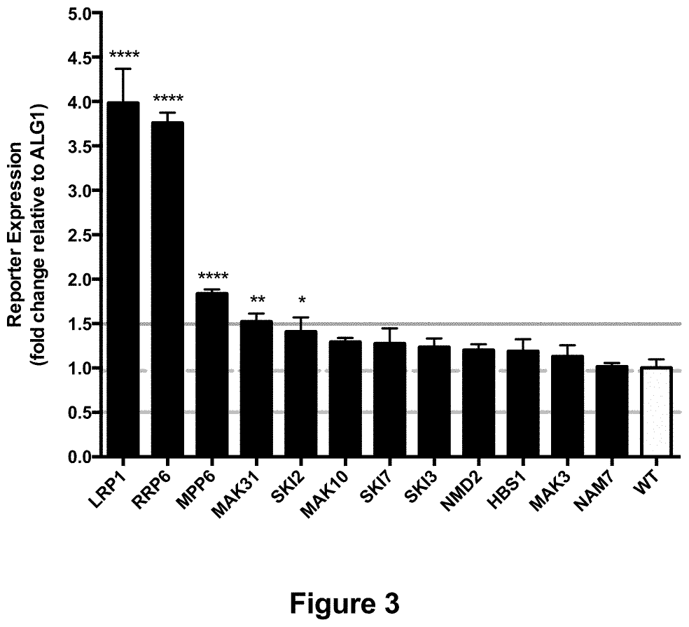

The most promising candidates—those with fold change in reporter expression less than 0.5 or greater than 1.5—were then analyzed in technical triplicate and normalized to the wild type Y7092 with genome integrated reporter gene construct (FIG. 3). A number of genes were identified that, when knocked out, resulted in statistically higher expression of the RNAi reporter construct. More specifically, disruption of RRP6, LRP1, and/or MPP6 (all essential components of the nuclear ribonucleic exosome complex), as well as members of the SKI ‘Super Killer’ (SKI2, SKI3, and SKI7) and MAK ‘Maintenance of Killer’ (MAK3, MAK10, MAK31) gene families resulted in 1.5-fold increases in reporter gene expression.

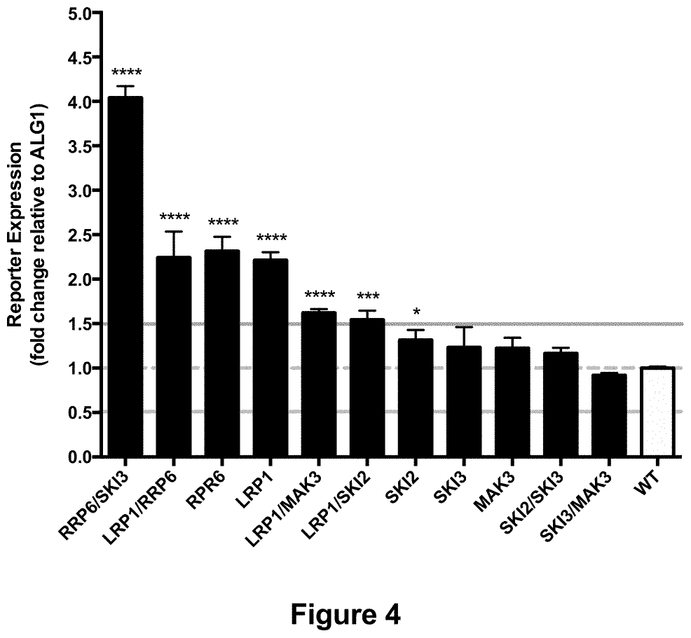

For select genes from this group (LRP1, RRP6, SKI2, SKI3, MAK3), double mutant haploid strains were constructed in which each strain contained one copy of the RNAi reporter construct, as well as two gene knockouts (FIG. 4). To generate the double mutants, hygromycin B resistance cassettes were amplified using primers (SEQ ID NO: 10, 11, 12, 13, 14, 15, 16, 17, 18, 19, 20, 21, 22, 23, 24, 25) flanked by 50 bp target gene homologous sequence. The purified DNA cassettes were transformed into selected single mutants using PEG3350/LiAc (Gietz & Schiestl, 2007). The transformants were selected on YEG plates containing both G418 and hygromycin and then confirmed genetically using primers upstream of target gene and inside antibiotic resistance gene (SEQ ID NO: 26 and 27). As seen in FIG. 3, one particular gene knockout combination (RRP6/SKI3) was observed that showed a >4-fold increase in reporter gene expression compared to wildtype and was substantially higher than either of the single knockout constituents.

In all experiments the effect of the gene knockouts on the expression of reporter construct appears to be specific, at least in part, to the reporter construct rather than affecting global transcription/RNA levels. This unexpected result, as evidenced by fold change in reporter gene expression >1 (relative to the transcription of the reference gene ACT1/ALG1), suggests, without wishing to be bound by theory, that specific combinations of gene knockouts are useful for facilitating the high-level expression of heterologous RNA constructs, such as RNAi effectors.

### Discussion

Taken together, the data shown in FIGS. 2, 3 and 4 indicate that the exploitation of gene knockouts can be used for the optimization of RNAi effector expression in yeast. In this way, it would be expected that such modifications could be purposed for the optimized expression of any RNAi effector sequence. Thus, such strain(s) would be platform strains applicable to any end use target including insects (e.g. agricultural pests, disease vectors), animals (e.g. livestock, aquaculture, and humans).

### Example 2: RNA Stability Penes

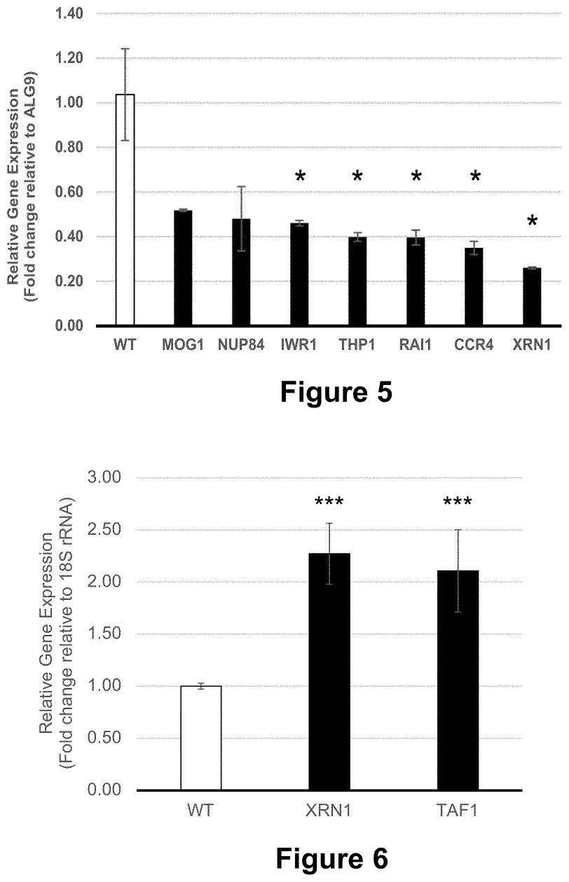

To identify RNA stability genes, a candidate set of seven deletion mutants involved in RNA stability were screened, each containing a genome-integrated RNAi effector reporter construct. Strains were screened by RT-qPCR, in which total RNA was extracted by hot acidic phenol-chloroform, purified, and reverse transcribed into cDNA. Quantitative PCR measuring RNAi effector reporter expression was performed using ALG9 as a housekeeping gene for normalization of data (SEQ ID NO: 30, 31, 71, 72). Results of the RT-qPCR screening are shown in FIG. 5.

Briefly, all of the deletion strains exhibited reporter expression at levels <50% (0.5× fold change), relative to a wild type strain containing the RNAi reporter construct. Of note, XRN1 showed the lowest levels of reporter gene expression at 26% of wild type (FIG. 5).

Based on this, XRN1 (which encodes a dual function mRNA stability factor) was chosen as an RNA stability gene for overexpression. In addition, TAF1, which could not be screened as a deletion due to its essential nature, was chosen to be overexpressed. TAF1 encodes a subunit of core general transcription factors and promotes RNA polymerase II transcription initiation.

To test the effect of RNA stability gene overexpression on RNAi effector expression, each of XRN1 and TAF1 was expressed from a strong constitutive promoter in wild type cells bearing a genome-integrated RNAi effector reporter construct. Strains were screened by RT-qPCR, in which total RNA was extracted by hot acidic phenol-chloroform, purified, and reverse transcribed into cDNA. Quantitative PCR was performed using 18S rRNA as a housekeeping gene (SEQ ID NO: 71, 72, 73 74). Results of the RT-qPCR screening are shown in FIG. 6.

Relative to wild type cells, cells overexpressing XRN1 and TAF1 expressed significantly more RNAi effector, with relative levels of the reporter increased by 2.27- and 2.11-fold, respectively. These data confirm the role of XRN1 and TAF1 as RNA stability genes suitable for overexpression to increase levels of RNAi effector molecule expression in yeast.

### Example 3: Plasmid Based RNAi Effector Expression

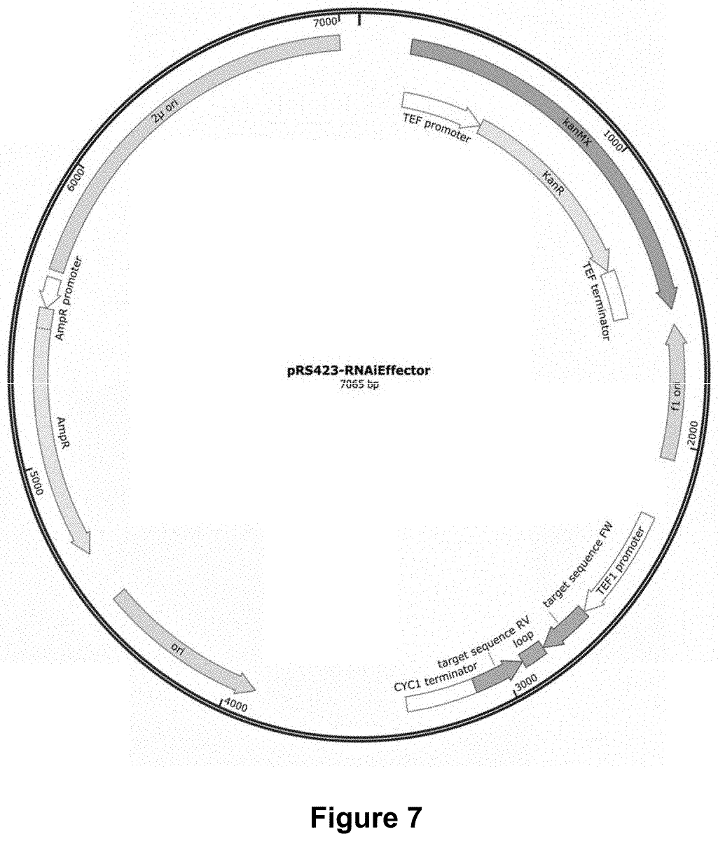

RNAi effectors may be expressed from genome integrated constructs or episomal (non-chromosomal) constructs, e.g. plasmids. To demonstrate the utility of a plasmid-based RNAi effector expression construct, a plasmid containing the same RNAi-effector construct as in the genome integrated version (FIG. 1A) was created, but carried on a high-copy (2 micron) plasmid instead (FIG. 7) (SEQ ID NO: 44).

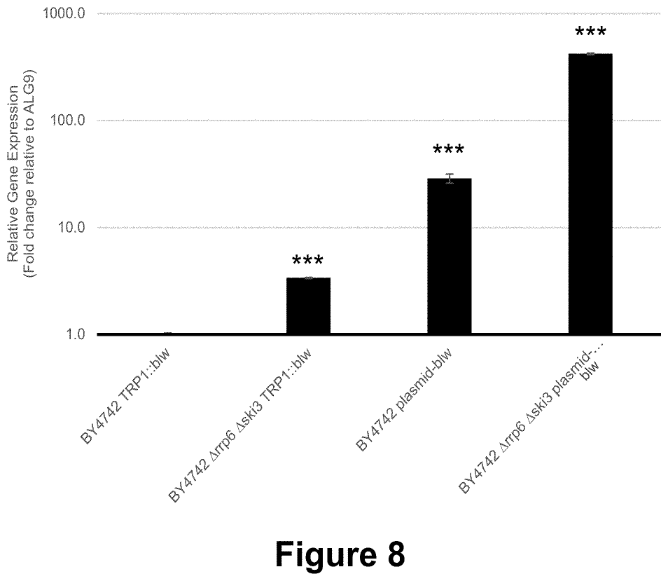

To test the plasmid-based RNAi-effector expression construct, as well as its interaction with previously identified RNA instability genes (RRP6/SKI3) that, when knocked out, were able to significantly upregulate genome-integrated RNAi effector reporter gene expression, the plasmid-based RNAi-effector expression construct was transformed into both wild type and Δrrp6/Δski3 cells. Resultant strains were screened by RT-qPCR, in which total RNA was extracted by hot acidic phenol-chloroform, purified, and reverse transcribed into cDNA. Quantitative PCR was performed using ALG9 as a housekeeping gene (SEQ ID NO: 30, 31, 59, 60). Results of the RT-qPCR screening are shown in FIG. 8.

As shown in FIG. 8, the Δrrp6/Δski3 mutation significantly increased integrated reporter gene expression by 3.4-fold, relative to wild type cells. On the other hand, the plasmid-based system expressed 28.8-fold more RNAi-effector reporter, relative to wild type cells. Lastly, the Δrrp6/Δski3 mutation synergistically increased expression of the plasmid-based reporter, by 420-fold and 14.6-fold compared to wild type cells with the integrated-construct and plasmid-construct, respectively (FIG. 8).

### Example 4: RNA Polymerase III-Based Expression

RNAi effectors may be expressed as different forms (e.g. siRNA, miRNA, dsRNA, shRNA, lhRNA, or anti-sense RNA). As such, RNAi effectors may be expressed from multiple different classes of cellular promoters, including RNA polymerase II promoters and RNA polymerase III promoters. As shown above, RNAi effectors may be expressed from both genome-integrated and plasmid-based RNA polymerase II promoter constructs (FIGS. 1-8) and RNA instability and stability gene modifications increase levels of RNAi effector expression (FIGS. 1-8).

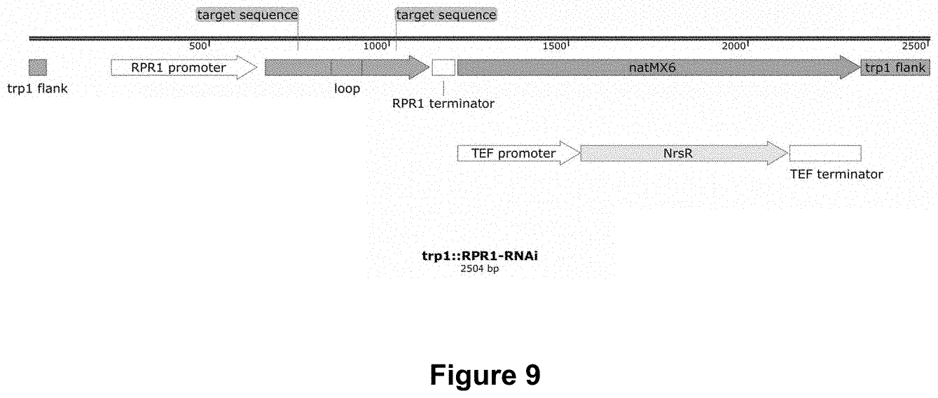

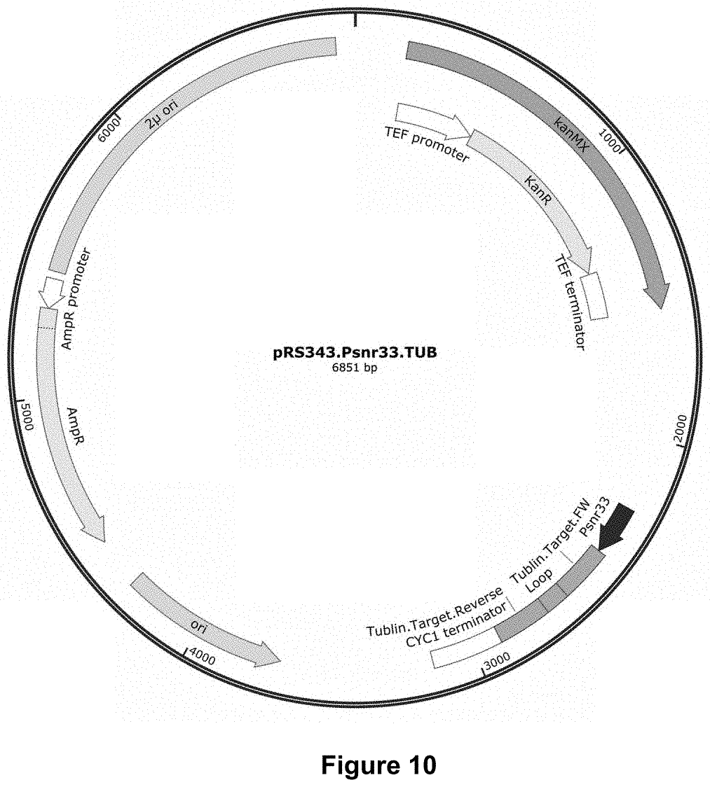

To demonstrate that RNAi effectors can also be expressed from RNA polymerase III promoters, expression constructs with either the yeast RPR1 (FIG. 9) and SNR33 (FIG. 10) promoters, both RNA polymerase III promoters, were constructed driving expression of an RNAi effector sequence (SEQ ID NO: 45 and 46). Of note, the yeast RPR1 gene encodes the RNA component of the nuclear RNase P Ribonucleoprotein, while the yeast SNR33 promoter encodes a small nucleolar protein involved in rRNA processing. RPR1 refers to RNase P Ribonucleoprotein 1 that may be from any yeast species or source, for example, S. cerevisiae or homologs thereof, and has the nucleic acid sequence as shown in Genbank Gene ID: 9164884 or SGD No. S000006490 or NCBI Reference Sequence: NR_132166.1. SNR33 refers to Small Nucleolar RNA 33 that may be from any yeast species or source, for example, S. cerevisiae or homologs thereof, and has the nucleic acid sequence as shown in Genbank Gene ID: 9164874 or SGD No. S000007298 or NCBI Reference Sequence: NR_132156.1.

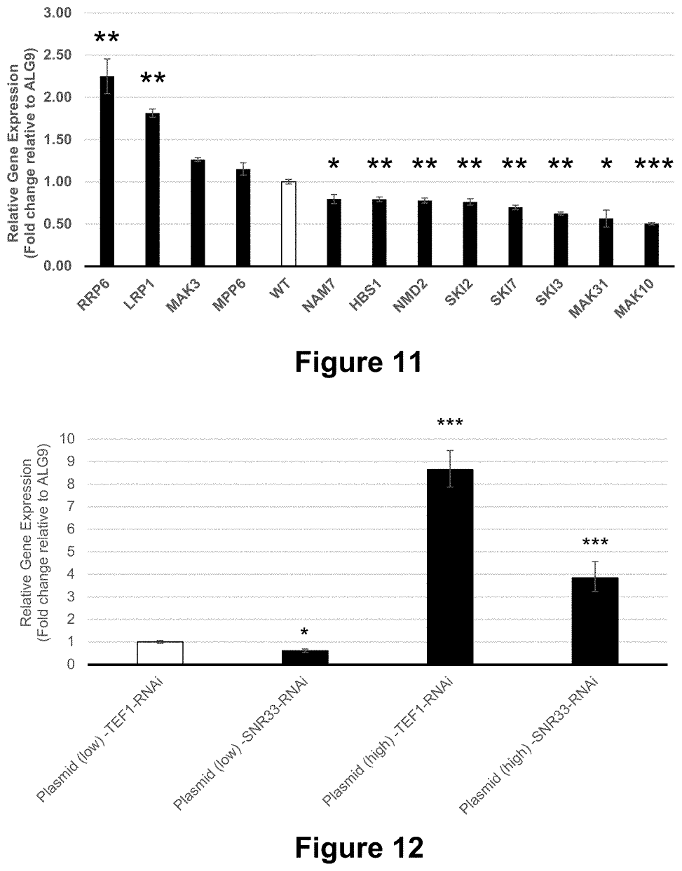

To test the RPR1 promoter-driven RNAi-effector expression construct, the construct was integrated into the yeast genome at the TRP1 locus. Using this reporter strain, a panel of 12 RNA instability and stability gene knockouts were screened in parallel with the wild type reporter strain to determine the effects of RNA stability/instability gene modifications on RPR1 promoter-driven RNAi-effector gene expression. Strains were screened by RT-qPCR, in which total RNA was extracted by hot acidic phenol-chloroform, purified, and reverse transcribed into cDNA. Quantitative PCR was performed using ALG9 as a housekeeping gene (SEQ ID NO: 30, 31, 71, 72). As shown in FIG. 11, the RPR1 RNA polymerase III promoter was suitable for expression of the RNAi effector, as reflected by detectable gene expression in wild type cells bearing the reporter. Furthermore, previously identified RNA instability gene modifications (i.e. LRP1 and RRP6) significantly upregulated reporter gene expression, 1.81- to 2.25-fold in this system, respectively (FIG. 11).

To test the SNR33 promoter-driven RNAi-effector expression construct, both low and high copy plasmids bearing the SNR33 promoter-driven RNAi-effector expression construct were created. These plasmids were transformed into wild type yeast strains alongside low and high copy plasmids with the previously characterized TEF1 promoter-driven RNAi-effector expression construct. Strains were screened by RT-qPCR, in which total RNA was extracted by hot acidic phenol-chloroform, purified, and reverse transcribed into cDNA. Quantitative PCR was performed using ALG9 as a housekeeping gene (SEQ ID NO: 30, 31, 71, 72). As shown in FIG. 12, in both the low and high copy plasmid constructs, the SNR33 promoter-driven RNAi-effector was able to be expressed to levels approximately 50% of that of the TEF1 promoter-driven RNAi-effector. This indicates that, like the RPR1 promoter, the SNR33 promoter is capable of expressing RNAi-effectors in yeast.

### Example 5: Applicability to Multiple, Distinct RNAi Effectors

Having demonstrated that RNAi effector genes can be expressed in yeast from either wild type or RNA instability/stability mutant cells (FIGS. 1-7), and from either a genome integrated or plasmid-based expression construct (FIG. 8), next the capacity of these systems to support high-level expression of a variety of different, biologically-relevant RNAi effectors was tested.

To do so, TEF1 promoter-driven RNAi-effector expression constructs were constructed for the following genes: bicoid (SEQ ID NO: 32) and bellwether (SEQ ID NO: 33) from Drosophila melanogaster, fez2 (SEQ ID NO: 34), gas8 (SEQ ID NO: 35), gnbpa1 (SEQ ID NO: 36), gnbpa3 (SEQ ID NO: 37), boule (SEQ ID NO: 38), and modsp (SEQ ID NO: 39) from Aedes aegypti, and IL1B-1 (SEQ ID NO: 40), IL1B-2 (SEQ ID NO: 41), and IL1B-3 (SEQ ID NO: 42) from Mus musculus, as well as EGFP (SEQ ID NO: 43). Using these constructs, both genome-integrated and plasmid-based versions were created that were then transformed into either wild type yeast cells or Δrrp6/Δski3 mutant yeast cells. All strains were screened by RT-qPCR, in which total RNA was extracted by hot acidic phenol-chloroform, purified, and reverse transcribed into cDNA. Quantitative PCR for each RNAi effector gene was performed using ALG9 as a housekeeping gene (SEQ ID 47, 48, 49, 50, 51, 52, 53, 54, 55, 56, 57, 58, 59, 60, 61, 62, 63, 64, 65, 66, 67, 68, 69, 70, 30, 31).

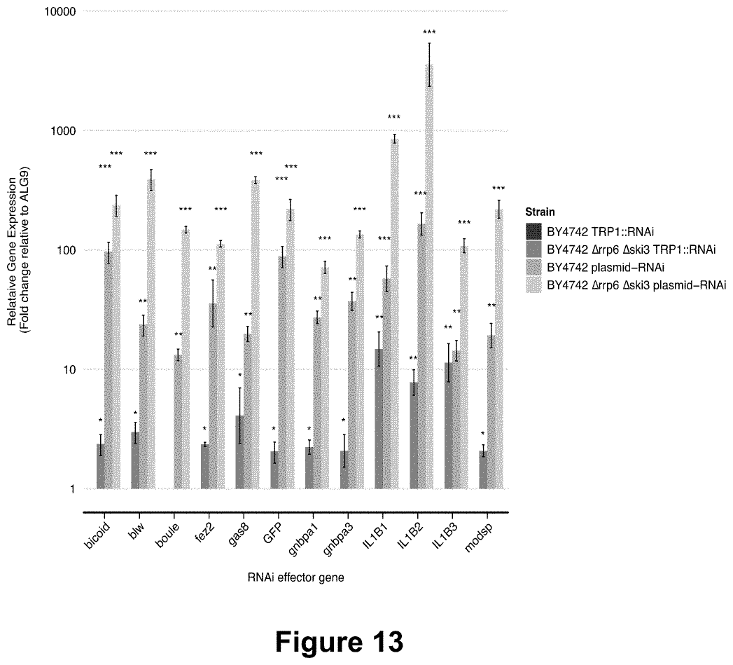

As shown in FIG. 13, for all RNAi effector genes, the Δrrp6/Δski3 mutant cells resulted in significantly higher levels of RNAi effector gene expression, relative to wild type cells. This was true for both genome-integrated and plasmid-based RNAi effector expression constructs. Across all RNAi effector genes, the average increase in expression level was 4.3- and 9.74-fold for the genome-integrated and plasmid-based expression constructs, respectively.

### Example 6: Feeding Based, Biological Activity in Insects: Drosophila Melanogaster

To test the insecticidal activity of the yeast-RNAi effector production and delivery system in an insect model system, D. melanogaster was used in a feeding and survival assay. Importantly D. melanogaster is a model organism for Dipteran insects and is closely related to a number of pest species, including Drosophila suzukii (Spotted Wing Drosophila) which is a fruit crop pest and is a serious economic threat to soft summer fruit (e.g. cherries, blueberries, raspberries, blackberries, peaches, nectarines, apricots, grapes, and others). D. suzukii has also been studied in the past with regards to RNAi based biocontrol (Murphy et al. 2016).

To induce an insecticidal effect in D. melanogaster, yeast expressing RNAi effectors targeting the D. melanogaster gene bellwether (blw) were developed (SEQ ID NO: 33). Bellwether encodes a subunit of the mitochondrial ATP synthase complex involved in the final enzymatic step of the oxidative phosphorylation pathway. It is an important protein involved in general energy metabolism and its knockdown was expected to have a detrimental effect in flies.

### Materials and Methods

### Fly Maintenance

The wildtype D. melanogaster strain used in this study was Canton-S. The flies were reared in standard cornmeal media at 25° C. under non-crowded conditions.

### Fly Feeding Experiments

Starvation media (SM-5% sucrose, 2% agarose) laced with yeast paste expressing RNAi targeting bellwether was used for the survival assay. Each vial had 5 mL of food and approximately 200 uL of yeast paste. Three different yeast strains were used in the experiment: BY4742 Δrrp6/Δski3, BY4742 plasmid-blw, and BY4742 Δrrp6/Δski3 plasmid-blw. For each treatment, 3 replicate vials of flies were used. For all the treatments, 20 black pupae were placed in each experimental vial containing starvation media (SM) and yeast paste (pupae were picked from food vials raised in standard cornmeal media). The flies eclosed (emerging as adults) within 24 hours from being placed in vials were flipped into fresh vials with SM and yeast. They were flipped every two days and the number of flies dying in each treatment was noted until their numbers sufficiently dwindled. The flies were always flipped into food vials with starvation media laced with the appropriate yeast paste. All the experiments were conducted in a humidity-controlled incubator at 25° C. in 12-hour light:dark cycle environment. Of note, fresh yeast paste was used at each stage to maintain consistency in yeast RNAi effector levels. Starvation media was also made fresh every time the flies were flipped.

Results

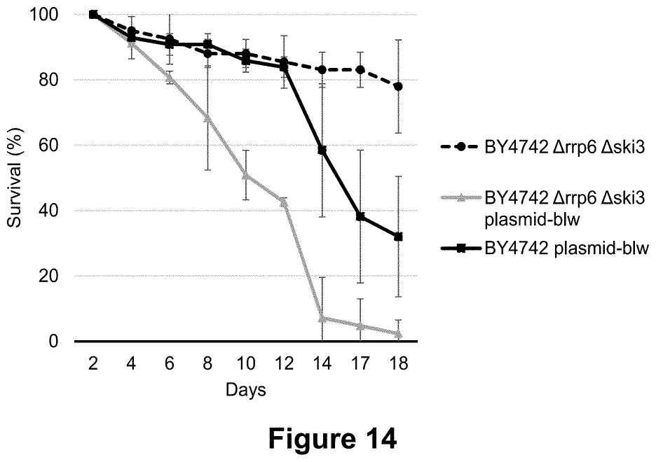

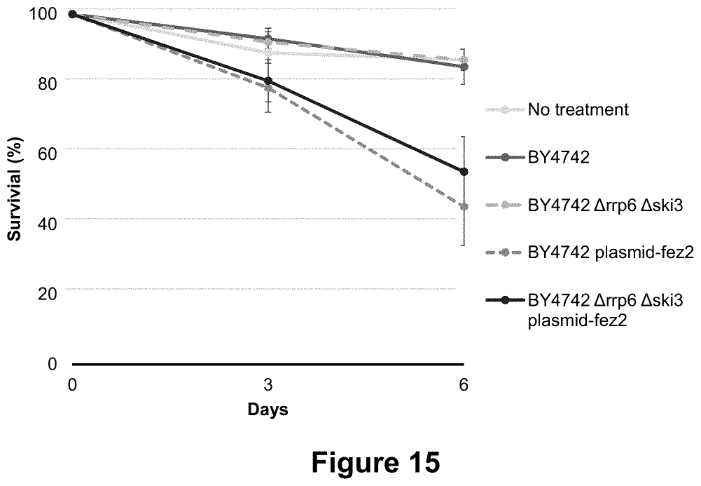

Adult D. melanogaster were fed ad libitum for 18 days with the following yeast strains, BY4742 Δrrp6/Δski3, BY4742 plasmid-blw, and BY4742 Δrrp6/Δski3 plasmid-blw. The number of live adults was determined at each timepoint (every 2 days), and percentage survival values were calculated relative to the starting point. As shown in FIG. 14, compared to the negative control not expressing blw RNAi (BY4742 Δrrp6/Δski3), both the yeast expressing blw RNAi (BY4742 plasmid-blw and BY4742 Δrrp6/Δski3 plasmid-blw) induced an insecticidal effect in the flies. Comparing the wildtype yeast expressing blw RNAi and the Δrrp6/Δski3 modified yeast expressing blw RNAi, we observed a substantial increase in insecticidal activity with <10% survival in BY4742 Δrrp6/Δski3 plasmid-blw treated flies after day 14 (as compared to nearly 60% survival in BY4742 plasmid-blw treated flies at this timepoint). At the end of the study (day 18), we observed <3% survival in BY4742 Δrrp6/Δski3 plasmid-blw treated flies, compared to 32% survival in BY4742 plasmid-blw treated flies (FIG. 14). Taken together these results indicate that yeast strains modified to increase levels of RNAi effectors by modulating levels of RNA stability and/or instability genes can be used successfully to elicit an insecticidal effect in D. melanogaster.

### Example 7: Feeding Based, Biological Activity in Insects: Aedes aegypti

### Introduction

The mosquito Aedes aegypti is a serious vector of a range of debilitating viruses, including dengue, chikungunya, and Zika virus. Currently, this mosquito is controlled by the application of broad-spectrum chemical pesticides. Through their overuse, many of these insecticides are no longer effective, as the mosquitoes have developed resistance. There are also increasing concerns about the negative impacts of these chemicals on non-target species. For these reasons, new environmentally-friendly insecticides must be developed.

A new range of species-specific pesticides is currently under development using double-stranded RNA (dsRNA). DsRNA, when it enters the cell, can induce sequence-specific knockdown of a targeted gene's expression. Because each species has its own unique gene sequences, dsRNAs that are designed to target genes essential for a mosquito's growth or development can potentially be used to selectively kill mosquitoes, without adversely affecting other species. This technology promises to dramatically reduce the environmental impact of mosquito larvicides. The high cost of producing dsRNA has prevented widespread adoption of RNAi, as have the challenges in stabilizing dsRNA against degradation in the aquatic environments where mosquitoes breed. To cheaply produce dsRNA stabilized within cells, yeast strains, in which high concentrations of dsRNA accumulate, were used. Yeasts are a typical food source for larval mosquitoes, and have been shown to be acceptable in dengue-endemic communities (Duman-Scheel et al. 2018).

RNAi can be used against a variety of gene targets in the mosquito, including genes specific to the brain (Hapairai et al. 2017) and cuticle (Lopez et al. 2019), and ubiquitously expressed genes (Whyard et al. 2009). In the experiments presented here, the neuronal gene fasciculation and elongation protein zeta 2 (fez2) was investigated, which has been used previously in a yeast-based RNAi insecticide formulation (Hapairai et al. 2017).

### Methods

### Hairpin RNA Construct Development

A yeast RNAi system was developed containing the constitutive strong promoter (TEF1), a short hairpin DNA sequence targeting Ae. aegypti fez2 (˜200 bp stem sequences and 74 bp loop sequence (SEQ ID NO: 34)), a CYC1 terminator, a NatMX resistance marker cassette and TRP1 flanking regions. This cassette was assembled in the expression vector pRS423-KanMX using Gibson cloning. All the fragments required for the Gibson reaction were PCR amplified and purified, excluding the backbone vector (digested by EcoRV) and reporter gene cassette (digested by Kpnl and Sall). The assembled 2.6 kb reporter system was harvested by restriction enzyme digestion (Bst1107Z) followed by DNA gel purification. The purified DNA fragment was used as donor DNA for integrating the reporter system into the TRP1 locus of the haploid laboratory S. cerevisiae strain BY4742.

The following strains were used in this study: BY4742, BY4742 Δrrp6 Δski3, BY4742 plasmid-fez2, BY4742 Δrrp6 Δski3 plasmid-fez2.

### Mosquito Rearing and Feeding

Ae. aegypti were reared under standard laboratory conditions with a 16:8 light: dark cycle and 65% humidity. Adults were fed on EDTA-treated rat blood, and eggs were collected on wet paper towels and kept moist in plastic bags. Eggs were hatched in boiled ddH₂O by bubbling nitrogen for 5 minutes. Within 2 hours of hatching, 40 larvae were placed in 90 mm petri dishes with 20 mL of ddH₂O and yeast feeding pellets.

Yeast feeding pellets were prepared by inoculating 7 mL of YEG media with a loopful of each strain and incubating with shaking overnight at 30° C. This starter broth was used to inoculate 300 mL of YEG media which was incubated overnight at 30° C. Cells were pelleted at 2000×g for 5 min and resuspended in 2.5 mL of 0.7% molten agar per gram of wet mass. This suspension was heat-killed at 80° C. for 20 minutes, vortexed briefly and poured into 10 mL open-barrel syringes. After solidification, two 0.5 mL feeding pellets were cut from the syringe using a clean cover-slip and added directly to petri dishes with mosquito larvae. For all treatments, fresh pellets were added on day 3.

Mortality was recorded on days 3 and 6 by removing mosquitoes from the water with a transfer pipette. Larvae were scored as dead if no movement was observed during processing. The developmental stage of each individual was also recorded.

### Results

Compared to two negative control yeast strains lacking fez2 RNAi effector expression (BY4742 and BY4742 Δrrp6 Δski3), as well as a no treatment control, increased mortality was observed in mosquitoes fed yeast strains expressing fez2 RNAi effectors (BY4742 plasmid-fez2 and BY4742 Δrrp6 Δski3 plasmid-fez2). In both cases, the wildtype and Δrrp6 Δski3 strains expressing fez2 RNAi effectors decreased survival of mosquitoes to approximately 50% (FIG. 14).

These findings indicate that yeast strains modified to increase levels of RNAi effectors by modulating levels of RNA stability and/or instability genes can be used successfully to elicit an insecticidal effect in Ae. aegypti. In this way, the modification of yeast strains does not negatively affect the functionality of the fez2 RNAi effector. It is noted however that the increased levels of fez2 RNAi effector expression observed previously in the Δrrp6 Δski3 yeast (FIG. 13—3.16 fold increase in expression in BY4742 Δrrp6 Δski3 plasmid-fez2 yeast relative to BY4742 plasmid-fez2 yeast), did not translate into increased insecticidal activity. Without wishing to be bound by theory, this is likely because the choice of gene target may have a major impact on the yeast RNAi effector expression levels required to induce an insecticidal effect. Indeed, fez2 is known to be highly insecticidal in mosquitos due to its highly essential gene function (Whyard et al. 2009). Therefore, it can be expected that even modest levels of knockdown would be capable of inducing an insecticidal effect. Further experimentation using lower doses of yeast expressing fez2 RNAi effectors, or indeed yeast targeting other less-critical genes, would be expected to demonstrate an improved insecticidal effect when comparing Δrrp6 Δski3 yeast and wildtype yeast.

### Example 8: Feeding Based, Biological Activity in Animals: Mus musculus and Inflammatory Bowel Disease

Inflammatory bowel disease (IBD), encompassing Crohn's disease and ulcerative colitis, is characterized by chronic, relapsing and remitting, or progressive inflammation of the intestine. Patients with IBD suffer from intestinal inflammation and experience symptoms such as pain, nausea, and diarrhea. Crohn's disease can occur in any part of the gastrointestinal tract, whereas ulcerative colitis is restricted to the colon. The immune response is critical in regulating host homeostasis during development of IBD and production of cytokines by immune cells contributes to intestinal inflammation. This is especially evident for production of the pro-inflammatory cytokine IL-1β, which is the master regulator of inflammation (Coccia et al. 2012). IL-1β plays a key role in the development of IBD by activating multiple types of immune cells. Progression of intestinal inflammation in patients with IBD is associated with increased levels of IL-1β production (Coccia et al. 2012). To better understand the effects of different factors involved in IBD pathogenesis, murine models have been used to model intestinal inflammation. Though none of the murine models available completely represent the features of IBD in humans, they have been crucial for investigating the contribution of various factors important for pathogenesis of IBD.

### A Mouse Model of Crohn's Disease-Like Intestinal Inflammation

The Src homology 2 domain-containing inositolpolyphosphate 5′-phosphatase (SHIP) is a hematopoietic-specific negative regulator of the phosphatidylinositol-3-kinase (PI3K) pathway. SHIP blunts PI3K activity by removing the 5′ phosphate group from class IA PI3K-generated phosphatidylinositol 3,4,5-triphosphate, an important second messenger in the cell membrane. SHIP expression levels and activity are reduced in the inflamed intestinal tissue from people with Crohn's disease. Similar to humans, SHIP deficient mice develop spontaneous intestinal Crohn's disease-like inflammation. Ileal inflammation is caused by increased production of macrophage-derived IL-1β (Ngoh et al. 2016).

### A Mouse Model Mimicking Ulcerative Colitis

The mucosa-associated lymphoid tissue lymphoma translocation 1 (MALT1) is a ubiquitously expressed protein and is one of the components critical for activation of NFκB, a family of inducible transcription factors that regulates the expression of a wide variety of genes involved in immune and inflammatory responses. Malt1 deficiency in humans causes dramatic inflammation along the gastrointestinal tract (McKinnon et al. 2014). Malt1 deficiency (Malt1^{-/-}) in mice does not result in spontaneous intestinal inflammation but it does exacerbate dextran sodium sulfate (DSS)-induced inflammation (Monajemi et al. 2018). Of note, DSS-induced colitis is a short-term, acute model of intestinal inflammation that occurs in the colon. DSS-induced colitis in Malt1^{-/-} mice is caused by increased production of IL-1β (Monajemi et al. 2018).

### Material and Methods

### DSS-Induced Colitis

Colitis was induced in Malt1^{-/-} mice by adding 2% DSS to their drinking water for 6 days. Mice were monitored daily to measure disease activity index (DAI). DAI was scored on a scale of 0-12 calculated as a sum of the 0-4 score for each of the following parameters: weight loss 0-4, stool consistency 0-4, and rectal bleeding 0-4. A score of 0=no weight loss, normal stool consistency, no rectal bleeding; 1=1-3% weight loss, loose stool, and detectable blood by HEMDETECT paper (Beckman Coulter, Mississauga, Canada); 2=3-6% weight loss, very loose stool, and visible blood in stool; 3=6-9% weight loss, diarrhoea, and occult blood in stool; and 4=more than 9% weight loss, no formed stool, and extensive blood in stool and blood visible at the anus. Colons were harvested from mice and fixed in 10% formalin overnight.

Long Hairpin RNA (lhRNA) Treatment

Malt1^{-/-} mice were orogastrically gavaged (feeding tube) with yeast containing lhRNA constructs (SEQ ID NO: 41) on days 0, 2, and 4 during development of DSS-induced colitis. SHIP^{-/-} were orogastrically gavaged on days 0 and 2 with a yeast concentration of 1×10⁹ yeast/mL and harvested on Day 10. SHIP^{-/-} were also orogastrically gavaged on days 0, 4, 8, and 12 with a yeast concentration of 2×10⁸ yeast/mL and harvested on Day 14. Administered fluid volumes of 5 mL/kg body weight were determined for each mouse. Control mice were gavaged with 5 mL/kg body weight of control yeast, as a vehicle control.

The following strains were used in this study: BY4742 Δrrp6 Δski3 empty plasmid (control), and BY4742 Δrrp6 Δski3 plasmid-IL1 B-2 (test).

### Histology Analysis

After autopsy, tissue sections were embedded in paraffin, and cross-sections were stained with H&E. Histological damage was scored using a 16-point scale by 2 individuals blinded to the experimental conditions. Scoring included: loss of architecture 0-4; immune cell infiltration 0-4; goblet cell depletion 0-2; ulceration 0-2; edema 0-2; and muscle thickening 0-2.

### Results

Yeast Harbouring lhRNA Decreased Histological Damage in SHIP Deficient Mice

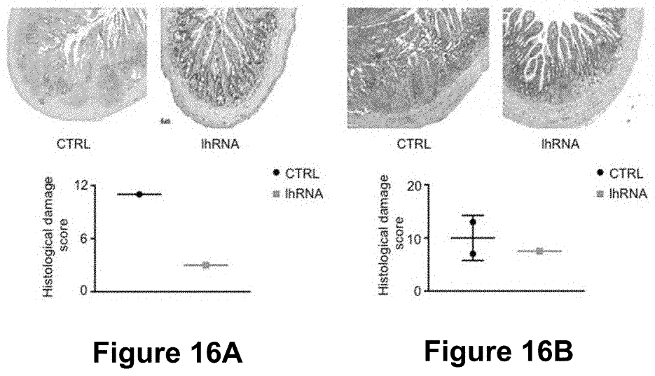

It was asked whether blocking IL-1β production by yeast containing lhRNA targeting IL-1β (SEQ ID NO: 41) in reduced development of spontaneous ileitis in SHIP deficient mice. Development of gross inflammation in the distal ileum of SHIP deficient mice is evident by at six weeks of age (McLarren et al. 2011). 6-week-old SHIP deficient mice were treated with yeast containing lhRNA for either 10 or 14 days. Ilea were fixed for histological analysis. Histological damage was scored by assessing loss of architecture, immune cell infiltration, goblet cell depletion, ulceration, edema 0-2, and muscle thickening. These facets were reduced in SHIP deficient mice treated with lhRNA compared to sham-treated mice (FIGS. 16A and 16B), thus resulting in a lower (better) histological damage score.

### LhRNA Targeting IL-1 f3 Ameliorates DSS-Induced Colitis in MeV Deficient Mice

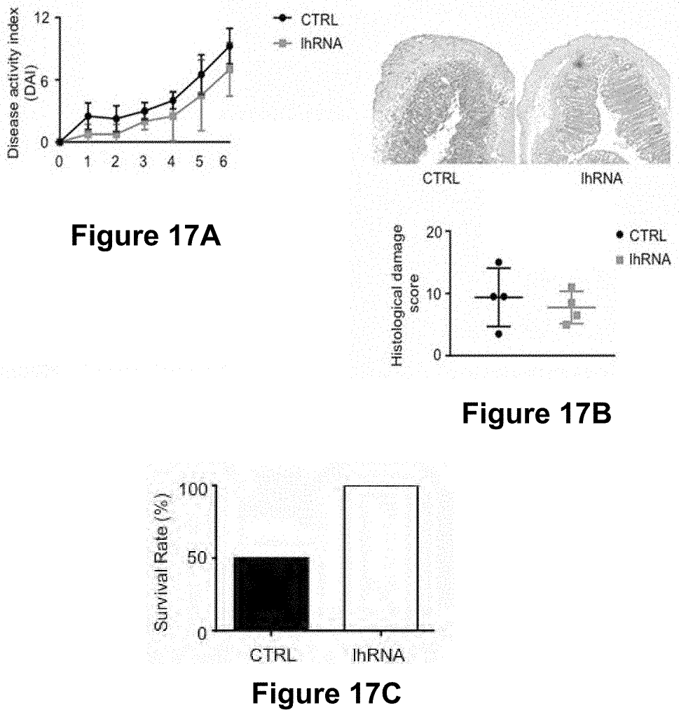

It was asked whether blocking IL-1β production by yeast containing lhRNA reduces DSS-induced intestinal inflammation in Malt1^{-/-} mice. Malt1^{-/-} mice were subjected to 2% DSS treatment to induce colitis and treated with yeast containing lhRNA (SEQ ID NO: 41) or control yeast. DAIs were monitored daily and after 6 days of treatment with DSS, mice were euthanized, and colons were harvested. Yeast containing lhRNA treatment decreased DAI modestly (FIG. 17A). Distal colons were fixed for histological analysis. There was also modest improvement in histological damage observed in mice treated with yeast containing lhRNA (FIG. 17B), as assessed by loss of architecture, immune cell infiltration, goblet cell depletion, ulceration, edema, and muscle thickening. Finally, survival rate was calculated based on humane endpoint (>15% weight loss) for Malt1^{-/-} mice treated with yeast containing lhRNA. Treatment with yeast containing lhRNA increased survival in Malt1^{-/-} mice compared to sham treated mice (FIG. 17C).

### Example 9: Another Example of Feeding Based, Biological Activity in Insects: Drosophila melanogaster

To evaluate the efficacy of RNAi effector delivery via engineered yeast strains to the fruit fly species Drosophila melanogaster, an eclosion assay was developed and implemented. The specific fly strain used was one in which the eGFP coding sequence (as described above in Example 5) was fused in-frame with the essential gene Neurexin IV (NrxIV::eGFP). (There are two transcript variants of NrxIV: isoform A=NM_079310.3 and isoform B=NM_168491.3; The eGFP trap construct was spliced into both isoforms. The location of the eGFP insertion is in a region which was spliced into both of them. The sequence difference between the two forms is fairly small); the transcripts produced in this gene fusion are translated into functional proteins, however contain the eGFP coding sequencing within them.

Materials and Methods

Developmental Rate Assay in Drosophila Melanogaster.

To collect eggs for the eclosion assay, crosses were set up between homozygous NrxIV::GFP females and males at a 2:1 ratio, with about 50 adults in each cage. Prior to egg collection, petri plates with grape juice medium coated with yeast paste (Fleischmann's active dry yeast #2192) were provided to acclimate the adults to the cage. The plates were replaced every day for three consecutive days. Three days later, flies were allowed to dump eggs onto a fresh yeast plate, 2h later the yeast plate was discarded (to ensure collection of stage-matched eggs), and replaced by a fresh yeast plate where cuts were made on the medium so as to increase surfaces available for egg laying (collection-plate).

Flies were allowed to lay eggs for 6 h before the collection-plate was withdrawn and eggs were collected and transferred into vials (95 mm height×25 mm diameter) containing ˜5 ml of 2% agar medium (with 0.1 g/100 ml methylparaben) laced with 200 ul of yeast samples (with 0.03 g/ml sucrose, 0.1 g/100 ml methylparaben and 6 ul/ml propionic acid). Using a brush, 50 eggs were transferred onto a small mesh. The mesh carrying 50 eggs, was subsequently placed onto the layer of yeast in each vial. The vials were placed at 25° C. and 70% humidity under a constant 12 h light/dark cycle. Starting from the 9th day after the egg collection, vials were monitored for the appearance of the first adult. Subsequently, the number of adult flies eclosing from each vial were counted every 12 h for the next three days.

Results

The target RNAi effector used in these assays was a 203 bp hairpin sequence targeting this eGFP sequence (eGFP long-hairpin), expressed in both WT BY4742 and BY4742 Δski3 Δrrp6 yeast. This is a TEF1 promoter-driven RNAi-effector expression construct for eGFP similar to the one used in Example 5 but only containing the first 203 nucleotides of SEQ ID NO:43.

For the eclosion assay, 50 stage-matched D. melanogaster eggs were placed on a layer of test yeast, on which they hatched and fed from for their entire life; the larvae proceeded through their life-cycle to form pupae, from which they then emerged (eclosed) as adult flies. In this assay, both the rate of eclosion and total number of adults eclosed was monitored over time.

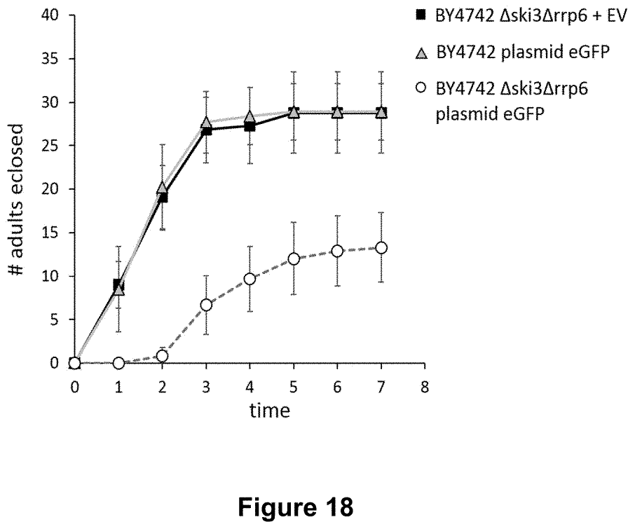

Flies raised on BY4742 Δski3 Δrrp6 yeast containing an empty expression plasmid (EV; negative control) had an average number of eclosed adult flies of ˜30, with the majority of eclosions occurring sharply within the first three timepoints. Similarly, flies raised on BY4742 containing the eGFP long-hairpin expressing plasmid showed a virtually identical eclosion rate and total number of eclosed adults to the negative control, whereas flies reared on optimized yeast, BY4742 Δski3 Δrrp6 containing the same eGFP long-hairpin expressing plasmid, showed a substantial decrease in the rate of eclosion, with flies emerging at later timepoints and more slowly overall, as well as >50% reduction of the total number of eclosed adults at the experimental endpoint, indicating a significant amount of insect death (FIG. 18). Overall, these results indicate that 1) biologically functional RNAi effector molecules can be delivered to D. melanogaster via feeding on S. cerevisiae yeast expressing a dsRNA and 2) in this model, the Δski3 Δrrp6 mutations, and subsequent increase in dsRNA levels in this strain, enables RNAi effector delivery function in this Dipteran species.

While the present disclosure has been described with reference to what are presently considered to be the examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Primers and sequences

| | | SEQ |
|---|---|---|
| Primer | Sequence | ID NO: |
| TRP up stream | GGTCGACGGTATCGATAAGCTTGATGTATACGTGTGCCCAATAGAAAGAGAAC | 3 |
| sequence with 343tail | | |
| FW | | |
| TRP up stream | GGAGTAGAAACATTTTGAAGCTATGGGTACCCTCCATGCAGTTGGACGATATC | 4 |
| sequence with reporter | | |
| tail RV | | |
| NatMX with reporter tail | TGGGACGCTCGAAGGCTTTAATTTGCGTCGACGACATGGAGGCCCAGAATAC | 5 |
| FW | | |
| NatMX with TRP down | CTTGGTATTCTTGCAGTATAGCGACCAGCATTC | 6 |
| stream tail RV | | |
| TRP down FW with | GGTCGCTATACTGCAAGAATACCAAGAGTTCCTCG | 7 |
| NatMX tail | | |
| TRP down RV with 343 | ATCCCCCGGGCTGCAGGAATTCGATGTATACCTCTTGCCTTCCAACCCAG | 8 |
| tail | | |
| guide RNA general RV | GATCATTTATCTTTCACTGCGGAG | 9 |
| TRP1 guide RNA FW | AACTGCATGGAGATGAGTCGGTTTTAGAGCTAGAAATAGCAAG | 10 |
| HPH with SKI3 tail FW | GTCAAGAAAGACACTAAGAACACAGAAAAGAAACACGAAGAGCAGAGGAAATGA | 11 |
| | CATGGAGGCCCAGAATAC | |
| HPH with SKI3 tail RV | GGGAAGTTTTCCAAATGGCATGATTACTCTATACAGCTGATAAACTCTGTCCAGT | 12 |
| | ATAGCGACCAGCATTCAC | |
| Ski3 confirmation FW | CCTGCGTAAGTTGGTTAAACG | 13 |
| HPH with MAK3 tail | GATTACAAGATAAAAAAGCCACTACTACAGAAAAGGCGTTGGGTCAGGACGACATGG | 14 |
| FW | AGGCCCAGAATAC | |
| HPH with MAK3 tail | GCTTTATTATCTCTCTCCTTTCTATTCCTCTTTTCTCTACTGCCCTTTTTCTCAGTAT | 15 |
| RV | AGCGACCAGCATTCAC | |
| MAK3 confirmation FW | GATAAAAAGGCTCTCCATGGC | 16 |
| HPH with LRP tail FW | GCCTACAGTAGAAATCGATTAATATAAACATATATCTAGCAACGTAACGGAGGACA | 17 |
| | TGGAGGCCCAGAATAC | |
| HPH with LRP tail RV | CTCACATCACCTTTAATCATTTTTTCACTCATGTACCAGTATACGTCGACCCAGTAT | 18 |
| | AGCGACCAGCATTCAC | |
| LRP confirmation FW | CGAATTCTCGTGCAGTTTTTC | 19 |
| RRP6 deletion HPH | GATAGACGAAATAGGAACAACAAACAGCTTATAAGCACCCAATAAGTGCGGACATG | 20 |
| with RPR 6 tail FW | GAGGCCCAGAATAC | |
| RRP6 deletion HPH | GCATGGGGGAGCCATAACTCCATGACACAGATATTCGATTAGATGAATTTAGCAGTA | 21 |
| with RPR 6 tail RV | TAGCGACCAGCATTCAC | |
| RRP6 confirmation | ACCCAAAAATATGAGGGCATC | 22 |
| primer FW | | |
| SKI2 deletion HPH with | GCCACATAGTTCTTTCCGATATGAACAACCTAACTCACAAAATTTACTGTACGA | 23 |
| SKi2 tail FW | CATGGAGGCCCAGAATAC | |
| SKI2 deletion HPH with | CTATGTATACGTGTGTGTGTGTGTGTGCAATAAGAGTTCGAAAACATTAACCA | 24 |
| SKi2 tail RV | GTATAGCGACCAGCATTCAC | |
| SKi2 confirmation | ATCACGACGGACGAAGTATTG | 25 |
| primer | | |
| KanMX_up_check RV | CCCATATAAATCAGCATCCATG | 26 |
| HgyMX_up_check RV | CGATCAGAAACTTCTCGACAG | 27 |
| ACT1_qPCR_Fwd | CGTCTGGATTGGTGGTTCTATC | 28 |
| ACT1_qPCR_Rev | GGACCACTTTCGTCGTATTCTT | 29 |
| ALG9 qPCR FW | CACGGATAGTGGCTTTGGTGAACAATTAC | 30 |
| ALG9 qPCR RV | TATGATTATCTGGCAGCAGGAAAGAACTTGGG | 31 |
| il1beta_1qpcr_F | TGAACTCAACTGTGAAATGCCAC | 47 |
| il1beta_lqpcr_R | TCATCAGGACAGCCCAGGTC | 48 |
| il1beta_2_qpcr_F | GTACAAGGAGAACCAAGCAACG | 49 |
| il1beta_2_qPCR_R | TAAGATTTCACACAGATCAGCCG | 50 |
| il1beta_3_qPCR_F | GGAAACAACAGTGGTCAGGACA | 51 |
| il1beta_3_qPCR_R | AGGAGTCCCCTGGAGATTGAG | 52 |
| Bicoid qPCR Fw | CCGAATCTGAAACAAATGGTCTG | 53 |
| Bicoid qPCR RV | CTATGCCAAAGTGTCTGACATAATC | 54 |
| BLWqPCR FW | TGTGGTCTTCGGTAACGATAAG | 55 |
| BLWqPCR RV | GACCCAGCAGCTCATCAC | 56 |
| EGFP qPCR FW | GCTGACCCTGAAGTTCATCT | 57 |
| EGFP qPCR RV | AGAAGTCGTGCTGCTTCAT | 58 |
| BouleF1 | GACCTCTTCCGCTGTCTTTATC | 59 |
| BouleR1 | CGTTACTCGGATCAGTAGTGTATTT | 60 |
| Gas8F1 | ATACTGGAGCTGCAACAGAAG | 61 |
| Gas8R1 | GACCTCTTCCGCTGTCTTTATC | 62 |
| Fez2F1 | GAAGATGAGGCCGTTGCTAA | 63 |
| Fez2R1 | GACCTCTTCCGCTGTCTTTATC | 64 |
| GNBPA1F1 | CGAGCATTTCAGCGATAACTTT | 65 |
| GNBPA1R1 | CTCAGGGTAGCATACTGGAATC | 66 |
| GNBPA3F1 | TGTCATATTGACCCGGAAGAAG | 67 |
| GNBPA3R1 | CGAACGGAGCCATTCGATTA | 68 |
| MODSPF1 | CTTACGTGTATCGACGGTTCTT | 69 |
| MODSPR1 | TGGCATTTCGACCTCCAATAA | 70 |
| tubulin qPCR FW | ACGGACGGTCAGGTACTAGC | 71 |
| tubulin qPCR RV | TGCGAGTCTTATAAACAATGTGCT | 72 |
| 18s_qPCR_F | CAGTGAAACTGCGAATGGC | 73 |
| 18s_qPCR_R | GAATCATCAAAGAGTCCGAAGAC | 74 |

SEQ ID NO: 1: RNAiEffector
ACTAGTGTGTGCCCAATAGAAAGAGAACAATTGACCCGGTTATTGCAAGGAAAATTTCAA
GTCTTGTAAAAGCATATAAAAATAGTTCAGGCACTCCGAAATACTTGGTTGGCGTGTTTC
GTAATCAACCTAAGGAGGATGTTTTGGCTCTGGTCAATGATTACGGCATTGATATCGTCC
AACTGCATGGAGGGTACCCATAGCTTCAAAATGTTTCTACTCCTTTTTTACTCTTCCAGAT
TTTCTCGGACTCCGCGCATCGCCGTACCACTTCAAAACACCCAAGCACAGCATACTAAAT
TTCCCCTCTTTCTTCCTCTAGGGTGTCGTTAATTACCCGTACTAAAGGTTTGGAAAAGAA
AAAAGAGACCGCCTCGTTTCTTTTTCTTCGTCGAAAAAGGCAATAAAAATTTTTATCACGT
TTCTTTTTCTTGAAAATTTTTTTTTTGATTTTTTTCTCTTTCGATGACCTCCCATTGATATTT
AAGTTAATAAACGGTCTTCAATTTCTCAAGTTTCAGTTTCATTTTTCTTGTTCTATTACAAC
TTTTTTTACTTCTTGCTCATTAGAAAGAAAGCATAGCAATCTAATCTAAGTCTAGAACGCT
AAGTCGGAGGACGGACGGTCAGGTACTAGCGGCGGTGTCTAGTTTGCTCTTGCCATCA
ACAATGCGTGCCATGCCTTTTCTCGAATGTATTTTACAATTTCTGAAGACGTCGGGATTG
GAAATCCCAAAGTATTAATAAGCACATTGTTTATAAGACTCGCATGTATGTTAATACTGTG
GATCCGTGAGTTTCTATTCGCAGTCGGCTGATCTGTGTGAAATCTTAATAAAGGGTCCAA
TTACCAATTTGAAACTCAGGAATTCACAGTATTAACATACATGCGAGTCTTATAAACAATG
TGCTTATTAATACTTTGGGATTTCCAATCCCGACGTCTTCAGAAATTGTAAAATACATTCG
AGAAAAGGCATGGCACGCATTGTTGATGGCAAGAGCAAACTAGACACCGCCGCTAGTAC
CTGACCGTCCGTCCTCCGACTTAGCGTAAGCTTTCATGTAATTAGTTATGTCACGCTTAC
ATTCACGCCCTCCCCCCACATCCGCTCTAACCGAAAAGGAAGGAGTTAGACAACCTGAA
GTCTAGGTCCCTATTTATTTTTTTATAGTTATGTTAGTATTAAGAACGTTATTTATATTTCAA
ATTTTTCTTTTTTTTCTGTACAGACGCGTGTACGCATGTAACATTATACTGAAAACCTTGC
TTGAGAAGGTTTTGGGACGCTCGAAGGCTTTAATTTGCGTCGACGGACATGGAGGCCCA
GAATACCCTCCTTGACAGTCTTGACGTGCGCAGCTCAGGGGCATGATGTGACTGTCGCC
CGTACATTTAGCCCATACATCCCCATGTATAATCATTTGCATCCATACATTTTGATGGCCG
CACGGCGCGAAGCAAAAATTACGGCTCCTCGCTGCAGACCTGCGAGCAGGGAAACGCT
CCCCTCACAGACGCGTTGAATTGTCCCCACGCCGCGCCCCTGTAGAGAAATATAAAAGG
TTAGGATTTGCCACTGAGGTTCTTCTTTCATATACTTCCTTTTAAAATCTTGCTAGGATAC
AGTTCTCACATCACATCCGAACATAAACAACCATGGGTACCACTCTTGACGACACGGCTT
ACCGGTACCGCACCAGTGTCCCGGGGGACGCCGAGGCCATCGAGGCACTGGATGGGT
CCTTCACCACCGACACCGTCTTCCGCGTCACCGCCACCGGGGACGGCTTCACCCTGCG
GGAGGTGCCGGTGGACCCGCCCCTGACCAAGGTGTTCCCCGACGACGAATCGGACGA
CGAATCGGACGACGGGGAGGACGGCGACCCGGACTCCCGGACGTTCGTCGCGTACGG
GGACGACGGCGACCTGGCGGGCTTCGTGGTCATCTCGTACTCGGCGTGGAACCGCCG
GCTGACCGTCGAGGACATCGAGGTCGCCCCGGAGCACCGGGGGCACGGGGTCGGGC
GCGCGTTGATGGGGCTCGCGACGGAGTTCGCCGGCGAGCGGGGCGCCGGGCACCTCT
GGCTGGAGGTCACCAACGTCAACGCACCGGCGATCCACGCGTACCGGCGGATGGGGT
TCACCCTCTGCGGCCTGGACACCGCCCTGTACGACGGCACCGCCTCGGACGGCGAGC
GGCAGGCGCTCTACATGAGCATGCCCTGCCCCTAATCAGTACTGACAATAAAAAGATTC
TTGTTTTCAAGAACTTGTCATTTGTATAGTTTTTTTATATTGTAGTTGTTCTATTTTAATCAA
ATGTTAGCGTGATTTATATTTTTTTTCGCCTCGACATCATCTGCCCAGATGCGAAGTTAAG
TGCGCAGAAAGTAATATCATGCGTCAATCGTATGTGAATGCTGGTCGCTATACTGGTCGA
CCAAGAATACCAAGAGTTCCTCGGTTTGCCAGTTATTAAAAGACTCGTATTTCCAAAAGA
CTGCAACATACTACTCAGTGCAGCTTCACAGAAACCTCATTCGTTTATTCCCTTGTTTGAT
TCAGAAGCAGGTGGGACAGGTGAACTTTTGGATTGGAACTCGATTTCTGACTGGGTTGG
AAGGCAAGAGGAGCTC
SEQ ID NO: 2: pRS423-RNAiEffector
TCGCGCGTTTCGGTGATGACGGTGAAAACCTCTGACACATGCAGCTCCCGGAGACGGT
CACAGCTTGTCTGTAAGCGGATGCCGGGAGCAGACAAGCCCGTCAGGGCGCGTCAGC
GGGTGTTGGCGGGTGTCGGGGCTGGCTTAACTATGCGGCATCAGAGCAGATTGTACTG
AGAGTGCACCATAGACATGGAGGCCCAGAATACCCTCCTTGACAGTCTTGACGTGCGCA
GCTCAGGGGCATGATGTGACTGTCGCCCGTACATTTAGCCCATACATCCCCATGTATAA
TCATTTGCATCCATACATTTTGATGGCCGCACGGCGCGAAGCAAAAATTACGGCTCCTC
GCTGCAGACCTGCGAGCAGGGAAACGCTCCCCTCACAGACGCGTTGAATTGTCCCCAC
GCCGCGCCCCTGTAGAGAAATATAAAAGGTTAGGATTTGCCACTGAGGTTCTTCTTTCAT
ATACTTCCTTTTAAAATCTTGCTAGGATACAGTTCTCACATCACATCCGAACATAAACAAC
CATGGGTAAGGAAAAGACTCACGTTTCGAGGCCGCGATTAAATTCCAACATGGATGCTG
ATTTATATGGGTATAAATGGGCTCGCGATAATGTCGGGCAATCAGGTGCGACAATCTATC
GATTGTATGGGAAGCCCGATGCGCCAGAGTTGTTTCTGAAACATGGCAAAGGTAGCGTT
GCCAATGATGTTACAGATGAGATGGTCAGACTAAACTGGCTGACGGAATTTATGCCTCTT
CCGACCATCAAGCATTTTATCCGTACTCCTGATGATGCATGGTTACTCACCACTGCGATC
CCCGGCAAAACAGCATTCCAGGTATTAGAAGAATATCCTGATTCAGGTGAAAATATTGTT
GATGCGCTGGCAGTGTTCCTGCGCCGGTTGCATTCGATTCCTGTTTGTAATTGTCCTTTT
AACAGCGATCGCGTATTTCGTCTCGCTCAGGCGCAATCACGAATGAATAACGGTTTGGT
TGATGCGAGTGATTTTGATGACGAGCGTAATGGCTGGCCTGTTGAACAAGTCTGGAAAG
AAATGCATAAGCTTTTGCCATTCTCACCGGATTCAGTCGTCACTCATGGTGATTTCTCAC
TTGATAACCTTATTTTTGACGAGGGGAAATTAATAGGTTGTATTGATGTTGGACGAGTCG
GAATCGCAGACCGATACCAGGATCTTGCCATCCTATGGAACTGCCTCGGTGAGTTTTCT
CCTTCATTACAGAAACGGCTTTTTCAAAAATATGGTATTGATAATCCTGATATGAATAAAT
TGCAGTTTCATTTGATGCTCGATGAGTTTTTCTAATCAGTACTGACAATAAAAAGATTCTT
GTTTTCAAGAACTTGTCATTTGTATAGTTTTTTTATATTGTAGTTGTTCTATTTTAATCAAAT
GTTAGCGTGATTTATATTTTTTTTCGCCTCGACATCATCTGCCCAGATGCGAAGTTAAGT
GCGCAGAAAGTAATATCATGCGTCAATCGTATGTGAATGCTGGTCGCTATACTGTATGCG
GTGTGAAATACCGCACAGATGCGTAAGGAGAAAATACCGCATCAGGAAATTGTAAACGT
TAATATTTTGTTAAAATTCGCGTTAAATTTTTGTTAAATCAGCTCATTTTTTAACCAATAGG
CCGAAATCGGCAAAATCCCTTATAAATCAAAAGAATAGACCGAGATAGGGTTGAGTGTTG
TTCCAGTTTGGAACAAGAGTCCACTATTAAAGAACGTGGACTCCAACGTCAAAGGGCGA
AAAACCGTCTATCAGGGCGATGGCCCACTACGTGAACCATCACCCTAATCAAGTTTTTTG
GGGTCGAGGTGCCGTAAAGCACTAAATCGGAACCCTAAAGGGAGCCCCCGATTTAGAG
CTTGACGGGGAAAGCCGGCGAACGTGGCGAGAAAGGAAGGGAAGAAAGCGAAAGGAG
CGGGCGCTAGGGCGCTGGCAAGTGTAGCGGTCACGCTGCGCGTAACCACCACACCCG
CCGCGCTTAATGCGCCGCTACAGGGCGCGTCGCGCCATTCGCCATTCAGGCTGCGCAA
CTGTTGGGAAGGGCGATCGGTGCGGGCCTCTTCGCTATTACGCCAGCTGGCGAAAGGG
GGATGTGCTGCAAGGCGATTAAGTTGGGTAACGCCAGGGTTTTCCCAGTCACGACGTTG
TAAAACGACGGCCAGTGAGCGCGCGTAATACGACTCACTATAGGGCGAATTGGGTACC
GGGCCCCCCCTCGAGGTCGACGGTATCGATAAGCTTGATACTAGTGTGTGCCCAATAGA
AAGAGAACAATTGACCCGGTTATTGCAAGGAAAATTTCAAGTCTTGTAAAAGCATATAAA
AATAGTTCAGGCACTCCGAAATACTTGGTTGGCGTGTTTCGTAATCAACCTAAGGAGGAT
GTTTTGGCTCTGGTCAATGATTACGGCATTGATATCGTCCAACTGCATGGAGGGTACCC
ATAGCTTCAAAATGTTTCTACTCCTTTTTTACTCTTCCAGATTTTCTCGGACTCCGCGCAT
CGCCGTACCACTTCAAAACACCCAAGCACAGCATACTAAATTTCCCCTCTTTCTTCCTCT
AGGGTGTCGTTAATTACCCGTACTAAAGGTTTGGAAAAGAAAAAAGAGACCGCCTCGTTT
CTTTTTCTTCGTCGAAAAAGGCAATAAAAATTTTTATCACGTTTCTTTTTCTTGAAAATTTT
TTTTTTGATTTTTTTCTCTTTCGATGACCTCCCATTGATATTTAAGTTAATAAACGGTCTTC
AATTTCTCAAGTTTCAGTTTCATTTTTCTTGTTCTATTACAACTTTTTTTACTTCTTGCTCAT
TAGAAAGAAAGCATAGCAATCTAATCTAAGTCTAGAACGCTAAGTCGGAGGACGGACGG
TCAGGTACTAGCGGCGGTGTCTAGTTTGCTCTTGCCATCAACAATGCGTGCCATGCCTT
TTCTCGAATGTATTTTACAATTTCTGAAGACGTCGGGATTGGAAATCCCAAAGTATTAATA
AGCACATTGTTTATAAGACTCGCATGTATGTTAATACTGTGGATCCGTGAGTTTCTATTCG
CAGTCGGCTGATCTGTGTGAAATCTTAATAAAGGGTCCAATTACCAATTTGAAACTCAGG
AATTCACAGTATTAACATACATGCGAGTCTTATAAACAATGTGCTTATTAATACTTTGGGA
TTTCCAATCCCGACGTCTTCAGAAATTGTAAAATACATTCGAGAAAAGGCATGGCACGCA
TTGTTGATGGCAAGAGCAAACTAGACACCGCCGCTAGTACCTGACCGTCCGTCCTCCGA
CTTAGCGTAAGCTTTCATGTAATTAGTTATGTCACGCTTACATTCACGCCCTCCCCCCAC
ATCCGCTCTAACCGAAAAGGAAGGAGTTAGACAACCTGAAGTCTAGGTCCCTATTTATTT
TTTTATAGTTATGTTAGTATTAAGAACGTTATTTATATTTCAAATTTTTCTTTTTTTTCTGTA
CAGACGCGTGTACGCATGTAACATTATACTGAAAACCTTGCTTGAGAAGGTTTTGGGAC
GCTCGAAGGCTTTAATTTGCGTCGACGGACATGGAGGCCCAGAATACCCTCCTTGACAG
TCTTGACGTGCGCAGCTCAGGGGCATGATGTGACTGTCGCCCGTACATTTAGCCCATAC
ATCCCCATGTATAATCATTTGCATCCATACATTTTGATGGCCGCACGGCGCGAAGCAAAA
ATTACGGCTCCTCGCTGCAGACCTGCGAGCAGGGAAACGCTCCCCTCACAGACGCGTT
GAATTGTCCCCACGCCGCGCCCCTGTAGAGAAATATAAAAGGTTAGGATTTGCCACTGA
GGTTCTTCTTTCATATACTTCCTTTTAAAATCTTGCTAGGATACAGTTCTCACATCACATC
CGAACATAAACAACCATGGGTACCACTCTTGACGACACGGCTTACCGGTACCGCACCAG
TGTCCCGGGGGACGCCGAGGCCATCGAGGCACTGGATGGGTCCTTCACCACCGACAC
CGTCTTCCGCGTCACCGCCACCGGGGACGGCTTCACCCTGCGGGAGGTGCCGGTGGA
CCCGCCCCTGACCAAGGTGTTCCCCGACGACGAATCGGACGACGAATCGGACGACGG
GGAGGACGGCGACCCGGACTCCCGGACGTTCGTCGCGTACGGGGACGACGGCGACCT
GGCGGGCTTCGTGGTCATCTCGTACTCGGCGTGGAACCGCCGGCTGACCGTCGAGGA
CATCGAGGTCGCCCCGGAGCACCGGGGGCACGGGGTCGGGCGCGCGTTGATGGGGC
TCGCGACGGAGTTCGCCGGCGAGCGGGGCGCCGGGCACCTCTGGCTGGAGGTCACCA
ACGTCAACGCACCGGCGATCCACGCGTACCGGCGGATGGGGTTCACCCTCTGCGGCCT
GGACACCGCCCTGTACGACGGCACCGCCTCGGACGGCGAGCGGCAGGCGCTCTACAT
GAGCATGCCCTGCCCCTAATCAGTACTGACAATAAAAAGATTCTTGTTTTCAAGAACTTG
TCATTTGTATAGTTTTTTTATATTGTAGTTGTTCTATTTTAATCAAATGTTAGCGTGATTTAT
ATTTTTTTTCGCCTCGACATCATCTGCCCAGATGCGAAGTTAAGTGCGCAGAAAGTAATA
TCATGCGTCAATCGTATGTGAATGCTGGTCGCTATACTGGTCGACCAAGAATACCAAGA
GTTCCTCGGTTTGCCAGTTATTAAAAGACTCGTATTTCCAAAAGACTGCAACATACTACTC
AGTGCAGCTTCACAGAAACCTCATTCGTTTATTCCCTTGTTTGATTCAGAAGCAGGTGGG
ACAGGTGAACTTTTGGATTGGAACTCGATTTCTGACTGGGTTGGAAGGCAAGAGGAGCT
CATCGAATTCCTGCAGCCCGGGGGATCCACTAGTTCTAGAGCGGCCGCCACCGCGGTG
GAGCTCCAGCTTTTGTTCCCTTTAGTGAGGGTTAATTGCGCGCTTGGCGTAATCATGGTC
ATAGCTGTTTCCTGTGTGAAATTGTTATCCGCTCACAATTCCACACAACATAGGAGCCGG
AAGCATAAAGTGTAAAGCCTGGGGTGCCTAATGAGTGAGGTAACTCACATTAATTGCGTT
GCGCTCACTGCCCGCTTTCCAGTCGGGAAACCTGTCGTGCCAGCTGCATTAATGAATCG
GCCAACGCGCGGGGAGAGGCGGTTTGCGTATTGGGCGCTCTTCCGCTTCCTCGCTCAC
TGACTCGCTGCGCTCGGTCGTTCGGCTGCGGCGAGCGGTATCAGCTCACTCAAAGGCG
GTAATACGGTTATCCACAGAATCAGGGGATAACGCAGGAAAGAACATGTGAGCAAAAGG
CCAGCAAAAGGCCAGGAACCGTAAAAAGGCCGCGTTGCTGGCGTTTTTCCATAGGCTCC
GCCCCCCTGACGAGCATCACAAAAATCGACGCTCAAGTCAGAGGTGGCGAAACCCGAC
AGGACTATAAAGATACCAGGCGTTTCCCCCTGGAAGCTCCCTCGTGCGCTCTCCTGTTC
CGACCCTGCCGCTTACCGGATACCTGTCCGCCTTTCTCCCTTCGGGAAGCGTGGCGCTT
TCTCATAGCTCACGCTGTAGGTATCTCAGTTCGGTGTAGGTCGTTCGCTCCAAGCTGGG
CTGTGTGCACGAACCCCCCGTTCAGCCCGACCGCTGCGCCTTATCCGGTAACTATCGTC
TTGAGTCCAACCCGGTAAGACACGACTTATCGCCACTGGCAGCAGCCACTGGTAACAGG
ATTAGCAGAGCGAGGTATGTAGGCGGTGCTACAGAGTTCTTGAAGTGGTGGCCTAACTA
CGGCTACACTAGAAGGACAGTATTTGGTATCTGCGCTCTGCTGAAGCCAGTTACCTTCG
GAAAAAGAGTTGGTAGCTCTTGATCCGGCAAACAAACCACCGCTGGTAGCGGTGGTTTT
TTTGTTTGCAAGCAGCAGATTACGCGCAGAAAAAAAGGATCTCAAGAAGATCCTTTGATC
TTTTCTACGGGGTCTGACGCTCAGTGGAACGAAAACTCACGTTAAGGGATTTTGGTCAT
GAGATTATCAAAAAGGATCTTCACCTAGATCCTTTTAAATTAAAAATGAAGTTTTAAATCAA
TCTAAAGTATATATGAGTAAACTTGGTCTGACAGTTACCAATGCTTAATCAGTGAGGCAC
CTATCTCAGCGATCTGTCTATTTCGTTCATCCATAGTTGCCTGACTCCCCGTCGTGTAGA
TAACTACGATACGGGAGGGCTTACCATCTGGCCCCAGTGCTGCAATGATACCGCGAGAC
CCACGCTCACCGGCTCCAGATTTATCAGCAATAAACCAGCCAGCCGGAAGGGCCGAGC
GCAGAAGTGGTCCTGCAACTTTATCCGCCTCCATCCAGTCTATTAATTGTTGCCGGGAA
GCTAGAGTAAGTAGTTCGCCAGTTAATAGTTTGCGCAACGTTGTTGCCATTGCTACAGGC
ATCGTGGTGTCACGCTCGTCGTTTGGTATGGCTTCATTCAGCTCCGGTTCCCAACGATC
AAGGCGAGTTACATGATCCCCCATGTTGTGCAAAAAAGCGGTTAGCTCCTTCGGTCCTC
CGATCGTTGTCAGAAGTAAGTTGGCCGCAGTGTTATCACTCATGGTTATGGCAGCACTG
CATAATTCTCTTACTGTCATGCCATCCGTAAGATGCTTTTCTGTGACTGGTGAGTACTCAA
CCAAGTCATTCTGAGAATAGTGTATGCGGCGACCGAGTTGCTCTTGCCCGGCGTCAATA
CGGGATAATACCGCGCCACATAGCAGAACTTTAAAAGTGCTCATCATTGGAAAACGTTCT
TCGGGGCGAAAACTCTCAAGGATCTTACCGCTGTTGAGATCCAGTTCGATGTAACCCAC
TCGTGCACCCAACTGATCTTCAGCATCTTTTACTTTCACCAGCGTTTCTGGGTGAGCAAA
AACAGGAAGGCAAAATGCCGCAAAAAAGGGAATAAGGGCGACACGGAAATGTTGAATAC
TCATACTCTTCCTTTTTCAATATTATTGAAGCATTTATCAGGGTTATTGTCTCATGAGCGG
ATACATATTTGAATGTATTTAGAAAAATAAACAAATAGGGGTTCCGCGCACATTTCCCCGA
AAAGTGCCACCTGAACGAAGCATCTGTGCTTCATTTTGTAGAACAAAAATGCAACGCGAG
AGCGCTAATTTTTCAAACAAAGAATCTGAGCTGCATTTTTACAGAACAGAAATGCAACGC
GAAAGCGCTATTTTACCAACGAAGAATCTGTGCTTCATTTTTGTAAAACAAAAATGCAACG
CGAGAGCGCTAATTTTTCAAACAAAGAATCTGAGCTGCATTTTTACAGAACAGAAATGCA
ACGCGAGAGCGCTATTTTACCAACAAAGAATCTATACTTCTTTTTTGTTCTACAAAAATGC
ATCCCGAGAGCGCTATTTTTCTAACAAAGCATCTTAGATTACTTTTTTTCTCCTTTGTGCG
CTCTATAATGCAGTCTCTTGATAACTTTTTGCACTGTAGGTCCGTTAAGGTTAGAAGAAG
GCTACTTTGGTGTCTATTTTCTCTTCCATAAAAAAAGCCTGACTCCACTTCCCGCGTTTAC
TGATTACTAGCGAAGCTGCGGGTGCATTTTTTCAAGATAAAGGCATCCCCGATTATATTC
TATACCGATGTGGATTGCGCATACTTTGTGAACAGAAAGTGATAGCGTTGATGATTCTTC
ATTGGTCAGAAAATTATGAACGGTTTCTTCTATTTTGTCTCTATATACTACGTATAGGAAA
TGTTTACATTTTCGTATTGTTTTCGATTCACTCTATGAATAGTTCTTACTACAATTTTTTTGT
CTAAAGAGTAATACTAGAGATAAACATAAAAAATGTAGAGGTCGAGTTTAGATGCAAGTT
CAAGGAGCGAAAGGTGGATGGGTAGGTTATATAGGGATATAGCACAGAGATATATAGCA
AAGAGATACTTTTGAGCAATGTTTGTGGAAGCGGTATTCGCAATATTTTAGTAGCTCGTT
ACAGTCCGGTGCGTTTTTGGTTTTTTGAAAGTGCGTCTTCAGAGCGCTTTTGGTTTTCAA
AAGCGCTCTGAAGTTCCTATACTTTCTAGAGAATAGGAACTTCGGAATAGGAACTTCAAA
GCGTTTCCGAAAACGAGCGCTTCCGAAAATGCAACGCGAGCTGCGCACATACAGCTCAC
TGTTCACGTCGCACCTATATCTGCGTGTTGCCTGTATATATATATACATGAGAAGAACGG
CATAGTGCGTGTTTATGCTTAAATGCGTACTTATATGCGTCTATTTATGTAGGATGAAAGG
TAGTCTAGTACCTCCTGTGATATTATCCCATTCCATGCGGGGTATCGTATGCTTCCTTCA
GCACTACCCTTTAGCTGTTCTATATGCTGCCACTCCTCAATTGGATTAGTCTCATCCTTCA
ATGCTATCATTTCCTTTGATATTGGATCATCTAAGAAACCATTATTATCATGACATTAACCT
ATAAAAATAGGCGTATCACGAGGCCCTTTCGTC
SEQ ID 32: Bicoid
AGTTATTCCGTTTGGCAGCAAAAAATCTCCGAATCTGAAACAAATGGTCT
GCATTGATTGAAAATACAATTTGCTGACTATTCTTGGTCAAAGAATGCGC
AAATGTTTGATTATGTCAGACACTTTGGCATAGCATAGAAATTGAAAATAT
CATATCAAATATTATTGTTTAAATGTTCGATCTTTAAGGGTAATCATTGGG
ATCCGTGAGTTTCTATTCGCAGTCGGCTGATCTGTGTGAAATCTTAATAA
AGGGTCCAATTACCAATTTGAAACTCAGGAATTCCAATGATTACCCTTAA
AGATCGAACATTTAAACAATAATATTTGATATGATATTTTCAATTTCTATGC
TATGCCAAAGTGTCTGACATAATCAAACATTTGCGCATTCTTTGACCAAG
AATAGTCAGCAAATTGTATTTTCAATCAATGCAGACCATTTGTTTCAGATT
CGGAGATTTTTTGCTGCCAAACGGAATAACT
SEQ ID 33: Bellwether
TTAACTTGGAGCCCGACAACGTCGGTGTTGTGGTCTTCGGTAACGATAA
GCTGATCAAGCAGGGCGATATCGTCAAGCGTACCGGTGCCATCGTGGAT
GTGCCCGTCGGTGATGAGCTGCTGGGTCGCGTCGTCGATGCCCTGGGA
AATGCCATCGACGGCAAGGGTGCCATCAACACCAAGGACCGTTTCCGTG
TGGGAATCAAGGGATCCGTGAGTTTCTATTCGCAGTCGGCTGATCTGTG
TGAAATCTTAATAAAGGGTCCAATTACCAATTTGAAACTCAGGAATTCCTT
GATTCCCACACGGAAACGGTCCTTGGTGTTGATGGCACCCTTGCCGTCG
ATGGCATTTCCCAGGGCATCGACGACGCGACCCAGCAGCTCATCACCG
ACGGGCACATCCACGATGGCACCGGTACGCTTGACGATATCGCCCTGCT
TGATCAGCTTATCGTTACCGAAGACCACAACACCGACGTTGTCGGGCTC
CAAGTTAA
SEQ ID 34: Fez2
CTCCGAAGATGAGGCCGTTGCTAACGATTTGGATATGCACGCATTGATT
CTGGGCGGCCTTCACACTGACAATGATCCGATAAAGACAGCGGAAGAG
GTCATCAAGGAAATTGACGATATTATGGACGAAAGCGCCTCCGAAGACG
GCATTGTTGGTAACGAAATCATGGAAAAAGCCAAAGAAGTTCTTGGATCT
CCCCGGATCCGTGAGTTTCTATTCGCAGTCGGCTGATCTGTGTGAAATC
TTAATAAAGGGTCCAATTACCAATTTGAAACTCAGGAATTCGGGGAGATC
CAAGAACTTCTTTGGCTTTTTCCATGATTTCGTTACCAACAATGCCGTCTT
CGGAGGCGCTTTCGTCCATAATATCGTCAATTTCCTTGATGACCTCTTCC
GCTGTCTTTATCGGATCATTGTCAGTGTGAAGGCCGCCCAGAATCAATG
CGTGCATATCCAAATCGTTAGCAACGGCCTCATCTTCGGAG
SEQ ID 35: gas8
CCTGCAGATGCGCTGCGAGAAGCTGGTCGAAGAACGCGATCAGCTGAA
GAATATGTTCGAGAAGTCTATACTGGAGCTGCAACAGAAGTCAGGTTTGA
AAAATTCCTTATTGGAGCGAAAACTAGAATACATCGAGAAGCAAACGGAA
CAACGGGAAGCCATTTTAGGGGAGGTGTTATCGCTTGCCGGAATCGAAC
CGCGGATCCGTGAGTTTCTATTCGCAGTCGGCTGATCTGTGTGAAATCTT
AATAAAGGGTCCAATTACCAATTTGAAACTCAGGAATTCGCGGTTCGATT
CCGGCAAGCGATAACACCTCCCCTAAAATGGCTTCCCGTTGTTCCGTTT
GCTTCTCGATGTATTCTAGTTTTCGCTCCAATAAGGAATTTTTCAAACCTG
ACTTCTGTTGCAGCTCCAGTATAGACTTCTCGAACATATTCTTCAGCTGA
TCGCGTTCTTCGACCAGCTTCTCGCAGCGCATCTGCAGG
SEQ ID 36: gnbpa1
CGAGCATTTCAGCGATAACTTTCATACCTATGGACTTGTGTGGAAGCCG
GACAGCATCGCTCTGACCGTGGATGGATTCCAGTATGCTACCCTGAGGG
ATCGGTTCAAGCCGTACGGTGCGGCCAACAATTTGACCCAGGCGAATTT
GTGGAATCCGGACAATGCCATGTCACCGTTTGATCGAGAGTTTTACATAT
CGCGGATCCGTGAGTTTCTATTCGCAGTCGGCTGATCTGTGTGAAATCTT
AATAAAGGGTCCAATTACCAATTTGAAACTCAGGAATTCGCGATATGTAA
AACTCTCGATCAAACGGTGACATGGCATTGTCCGGATTCCACAAATTCGC
CTGGGTCAAATTGTTGGCCGCACCGTACGGCTTGAACCGATCCCTCAGG
GTAGCATACTGGAATCCATCCACGGTCAGAGCGATGCTGTCCGGCTTCC
ACACAAGTCCATAGGTATGAAAGTTATCGCTGAAATGCTCG
SEQ ID 37: gnbpa3
CCCGGAAGGAGTGTACATGGAAGTGGACGATGAAGTGTACTGTCATATT
GACCCGGAAGAAGGCTTCTACAACGAGGTGAAAGCGACGAAACCGCAA
TTTGCAAACCTTTGGAGATTGAGCGGTAATCGAATGGCTCCGTTCGATAA
GGAGTTCTTCATTAGTTTGGGCGTCGGTGTGGGTGGTCACTACGACTTC
CACCGGATCCGTGAGTTTCTATTCGCAGTCGGCTGATCTGTGTGAAATCT
TAATAAAGGGTCCAATTACCAATTTGAAACTCAGGAATTCGGTGGAAGTC
GTAGTGACCACCCACACCGACGCCCAAACTAATGAAGAACTCCTTATCG
AACGGAGCCATTCGATTACCGCTCAATCTCCAAAGGTTTGCAAATTGCG
GTTTCGTCGCTTTCACCTCGTTGTAGAAGCCTTCTTCCGGGTCAATATGA
CAGTACACTTCATCGTCCACTTCCATGTACACTCCTTCCGGG
SEQ ID 38: boule
AACCATTGTTGAGCGATATTATCATTATTACACTAGTGATCATATTATAAC
TTATTAACAAACTATTTGTAGCGTAGTGATGATGGAGAGAGGAGTATCGA
AGAAGAGGCAGGAGAAGCAAGTCAGATAAATATTAGGAAAGTATGCGAA
AAACACGTGAATAAAAAAAATACACTACTGATCCGAGTAACGGTAGCTGG
GGATCCGTGAGTTTCTATTCGCAGTCGGCTGATCTGTGTGAAATCTTAAT
AAAGGGTCCAATTACCAATTTGAAACTCAGGAATTCCCAGCTACCGTTAC
TCGGATCAGTAGTGTATTTTTTTTATTCACGTGTTTTTCGCATACTTTCCT
AATATTTATCTGACTTGCTTCTCCTGCCTCTTCTTCGATACTCCTCTCTCC
ATCATCACTACGCTACAAATAGTTTGTTAATAAGTTATAATATGATCACTA
GTGTAATAATGATAATATCGCTCAACAATGGTT
SEQ ID 39: modsp
TGAAACTCTTACGTGTATCGACGGTTCTTGGGACAGTTCAGTGTTTCGAT
GTGAGCCCACCTGTGGAACACCAACGCCAGATGCTGAAGCATACATTAT
TGGAGGTCGAAATGCCACCATAACGGAGGTCCCATGGCATACTGGAATA
TATCGAAATCTGGAAACAGACACCATCGAAGATCTTCGATCAGAAGATTG
GCGGATCCGTGAGTTTCTATTCGCAGTCGGCTGATCTGTGTGAAATCTTA
ATAAAGGGTCCAATTACCAATTTGAAACTCAGGAATTCGCCAATCTTCTG
ATCGAAGATCTTCGATGGTGTCTGTTTCCAGATTTCGATATATTCCAGTAT
GCCATGGGACCTCCGTTATGGTGGCATTTCGACCTCCAATAATGTATGCT
TCAGCATCTGGCGTTGGTGTTCCACAGGTGGGCTCACATCGAAACACTG
AACTGTCCCAAGAACCGTCGATACACGTAAGAGTTTCA
SEQ ID 40: IL-1β-1
TGAACTCAACTGTGAAATGCCACCTTTTGACAGTGATGAGAATGACCTGT
TCTTTGAAGTTGACGGACCCCAAAAGATGAAGGGCTGCTTCCAAACCTTT
GACCTGGGCTGTCCTGATGAGAGCATCCAGCTTCAAATCTCGCAGCAGC
ACATCAACAAGAGCTTCAGGCAGGCAGTATCACTCATTGTGGCTGTGGA
GAGGATCCGTGAGTTTCTATTCGCAGTCGGCTGATCTGTGTGAAATCTTA
ATAAAGGGTCCAATTACCAATTTGAAACTCAGGAATTCTCTCCACAGCCA
CAATGAGTGATACTGCCTGCCTGAAGCTCTTGTTGATGTGCTGCTGCGA
GATTTGAAGCTGGATGCTCTCATCAGGACAGCCCAGGTCAAAGGTTTGG
AAGCAGCCCTTCATCTTTTGGGGTCCGTCAACTTCAAAGAACAGGTCATT
CTCATCACTGTCAAAAGGTGGCATTTCACAGTTGAGTTCA
SEQ ID 41: IL-1β-2
GCTCCGAGATGAACAACAAAAAAGCCTCGTGCTGTCGGACCCATATGAG
CTGAAAGCTCTCCACCTCAATGGACAGAATATCAACCAACAAGTGATATT
CTCCATGAGCTTTGTACAAGGAGAACCAAGCAACGACAAAATACCTGTG
GCCTTGGGCCTCAAAGGAAAGAATCTATACCTGTCCTGTGTAATGAAAGA
CGGGATCCGTGAGTTTCTATTCGCAGTCGGCTGATCTGTGTGAAATCTTA
ATAAAGGGTCCAATTACCAATTTGAAACTCAGGAATTCCGTCTTTCATTAC
ACAGGACAGGTATAGATTCTTTCCTTTGAGGCCCAAGGCCACAGGTATTT
TGTCGTTGCTTGGTTCTCCTTGTACAAAGCTCATGGAGAATATCACTTGT
TGGTTGATATTCTGTCCATTGAGGTGGAGAGCTTTCAGCTCATATGGGTC
CGACAGCACGAGGCTTTTTTGTTGTTCATCTCGGAGC
SEQ ID 42: IL-1β-3
GTCTTCCTGGGAAACAACAGTGGTCAGGACATAATTGACTTCACCATGG
AATCCGTGTCTTCCTAAAGTATGGGCTGGACTGTTTCTAATGCCTTCCCC
AGGGCATGTTAAGGAGCTCCCTTTTCGTGAATGAGCAGACAGCTCAATC
TCCAGGGGACTCCTTAGTCCTCGGCCAAGACAGGTCGCTCAGGGTCAC
AAGAGGATCCGTGAGTTTCTATTCGCAGTCGGCTGATCTGTGTGAAATCT
TAATAAAGGGTCCAATTACCAATTTGAAACTCAGGAATTCTCTTGTGACC
CTGAGCGACCTGTCTTGGCCGAGGACTAAGGAGTCCCCTGGAGATTGA
GCTGTCTGCTCATTCACGAAAAGGGAGCTCCTTAACATGCCCTGGGGAA
GGCATTAGAAACAGTCCAGCCCATACTTTAGGAAGACACGGATTCCATG
GTGAAGTCAATTATGTCCTGACCACTGTTGTTTCCCAGGAAGAC
SEQ ID 43: EGFP
CGGCCACAAGTTCAGCGTGTCCGGCGAGGGCGAGGGCGATGCCACCTA
CGGCAAGCTGACCCTGAAGTTCATCTGCACCACCGGCAAGCTGCCCGT
GCCCTGGCCCACCCTCGTGACCACCCTGACCTACGGCGTGCAGTGCTT
CAGCCGCTACCCCGACCACATGAAGCAGCACGACTTCTTCAAGTCCGCC
ATGCCCGAAGGGATCCGTGAGTTTCTATTCGCAGTCGGCTGATCTGTGT
GAAATCTTAATAAAGGGTCCAATTACCAATTTGAAACTCAGGAATTCCTTC
GGGCATGGCGGACTTGAAGAAGTCGTGCTGCTTCATGTGGTCGGGGTA
GCGGCTGAAGCACTGCACGCCGTAGGTCAGGGTGGTCACGAGGGTGG
GCCAGGGCACGGGCAGCTTGCCGGTGGTGCAGATGAACTTCAGGGTCA
GCTTGCCGTAGGTGGCATCGCCCTCGCCCTCGCCGGACACGCTGAACT
TGTGGCCG
SEQ ID 44: pRS423-HC-RNAiEffector
TCGCGCGTTTCGGTGATGACGGTGAAAACCTCTGACACATGCAGCTCCC
GGAGACGGTCACAGCTTGTCTGTAAGCGGATGCCGGGAGCAGACAAGC
CCGTCAGGGCGCGTCAGCGGGTGTTGGCGGGTGTCGGGGCTGGCTTA
ACTATGCGGCATCAGAGCAGATTGTACTGAGAGTGCACCATAGGTTAGG
ATTTGCCACTGAGGTTCTTCTTTCATATACTTCCTTTTAAAATCTTGCTAG
GATACAGTTCTCACATCACATCCGAACATAAACAACCATGGGTAAGGAAA
AGACTCACGTTTCGAGGCCGCGATTAAATTCCAACATGGATGCTGATTTA
TATGGGTATAAATGGGCTCGCGATAATGTCGGGCAATCAGGTGCGACAA
TCTATCGATTGTATGGGAAGCCCGATGCGCCAGAGTTGTTTCTGAAACAT
GGCAAAGGTAGCGTTGCCAATGATGTTACAGATGAGATGGTCAGACTAA
ACTGGCTGACGGAATTTATGCCTCTTCCGACCATCAAGCATTTTATCCGT
ACTCCTGATGATGCATGGTTACTCACCACTGCGATCCCCGGCAAAACAG
CATTCCAGGTATTAGAAGAATATCCTGATTCAGGTGAAAATATTGTTGAT
GCGCTGGCAGTGTTCCTGCGCCGGTTGCATTCGATTCCTGTTTGTAATT
GTCCTTTTAACAGCGATCGCGTATTTCGTCTCGCTCAGGCGCAATCACG
AATGAATAACGGTTTGGTTGATGCGAGTGATTTTGATGACGAGCGTAATG
GCTGGCCTGTTGAACAAGTCTGGAAAGAAATGCATAAGCTTTTGCCATTC
TCACCGGATTCAGTCGTCACTCATGGTGATTTCTCACTTGATAACCTTATT
TTTGACGAGGGGAAATTAATAGGTTGTATTGATGTTGGACGAGTCGGAAT
CGCAGACCGATACCAGGATCTTGCCATCCTATGGAACTGCCTCGGTGAG
TTTTCTCCTTCATTACAGAAACGGCTTTTTCAAAAATATGGTATTGATAAT
CCTGATATGAATAAATTGCAGTTTCATTTGATGCTCGATGAGTTTTTCTAA
TCAGTACTGACAATAAAAAGATTCTTGTTTTCAAGAACTTGTCATTTGTAT
AGTTTTTTTATATTGTAGTTGTTCTATTTTAATCAAATGTTAGCGTGATTTA
TATTTTTTTTCGCCTCGACATCATCTGCCCAGATGCGAAGTTAAGTGCGC
AGAAAGTAATATCATGCGTCAATCGTATGTGAATGCTGGTCGCTATACTG
TATGCGGTGTGAAATACCGCACAGATGCGTAAGGAGAAAATACCGCATC
AGGAAATTGTAAACGTTAATATTTTGTTAAAATTCGCGTTAAATTTTTGTTA
AATCAGCTCATTTTTTAACCAATAGGCCGAAATCGGCAAAATCCCTTATAA
ATCAAAAGAATAGACCGAGATAGGGTTGAGTGTTGTTCCAGTTTGGAACA
AGAGTCCACTATTAAAGAACGTGGACTCCAACGTCAAAGGGCGAAAAAC
CGTCTATCAGGGCGATGGCCCACTACGTGAACCATCACCCTAATCAAGT
TTTTTGGGGTCGAGGTGCCGTAAAGCACTAAATCGGAACCCTAAAGGGA
GCCCCCGATTTAGAGCTTGACGGGGAAAGCCGGCGAACGTGGCGAGAA
AGGAAGGGAAGAAAGCGAAAGGAGCGGGCGCTAGGGCGCTGGCAAGT
GTAGCGGTCACGCTGCGCGTAACCACCACACCCGCCGCGCTTAATGCG
CCGCTACAGGGCGCGTCGCGCCATTCGCCATTCAGGCTGCGCAACTGT
TGGGAAGGGCGATCGGTGCGGGCCTCTTCGCTATTACGCCAGCTGGCG
AAAGGGGGATGTGCTGCAAGGCGATTAAGTTGGGTAACGCCAGGGTTTT
CCCAGTCACGACGTTGTAAAACGACGGCCAGTGAGCGCGCGTAATACG
ACTCACTATAGGGCGAATTGGGTACCATAGCTTCAAAATGTTTCTACTCC
TTTTTTACTCTTCCAGATTTTCTCGGACTCCGCGCATCGCCGTACCACTT
CAAAACACCCAAGCACAGCATACTAAATTTCCCCTCTTTCTTCCTCTAGG
GTGTCGTTAATTACCCGTACTAAAGGTTTGGAAAAGAAAAAAGAGACCGC
CTCGTTTCTTTTTCTTCGTCGAAAAAGGCAATAAAAATTTTTATCACGTTT
CTTTTTCTTGAAAATTTTTTTTTTGATTTTTTTCTCTTTCGATGACCTCCCA
TTGATATTTAAGTTAATAAACGGTCTTCAATTTCTCAAGTTTCAGTTTCATT
TTTCTTGTTCTATTACAACTTTTTTTACTTCTTGCTCATTAGAAAGAAAGCA
TAGCAATCTAATCTAAGTCTAGAACGCTAAGTCGGAGGACGGACGGTCA
GGTACTAGCGGCGGTGTCTAGTTTGCTCTTGCCATCAACAATGCGTGCC
ATGCCTTTTCTCGAATGTATTTTACAATTTCTGAAGACGTCGGGATTGGA
AATCCCAAAGTATTAATAAGCACATTGTTTATAAGACTCGCATGTATGTTA
ATACTGTGGATCCGTGAGTTTCTATTCGCAGTCGGCTGATCTGTGTGAAA
TCTTAATAAAGGGTCCAATTACCAATTTGAAACTCAGGAATTCACAGTATT
AACATACATGCGAGTCTTATAAACAATGTGCTTATTAATACTTTGGGATTT
CCAATCCCGACGTCTTCAGAAATTGTAAAATACATTCGAGAAAAGGCATG
GCACGCATTGTTGATGGCAAGAGCAAACTAGACACCGCCGCTAGTACCT
GACCGTCCGTCCTCCGACTTAGCGTAAGCTTTCATGTAATTAGTTATGTC
ACGCTTACATTCACGCCCTCCCCCCACATCCGCTCTAACCGAAAAGGAA
GGAGTTAGACAACCTGAAGTCTAGGTCCCTATTTATTTTTTTATAGTTATG
TTAGTATTAAGAACGTTATTTATATTTCAAATTTTTCTTTTTTTTCTGTACAG
ACGCGTGTACGCATGTAACATTATACTGAAAACCTTGCTTGAGAAGGTTT
TGGGACGCTCGAAGGCTTTAATTTGCGTCGACGGTATCGATAAGCTTGA
TATCGAATTCCTGCAGCCCGGGGGATCCACTAGTTCTAGAGCGGCCGCC
ACCGCGGTGGAGCTCCAGCTTTTGTTCCCTTTAGTGAGGGTTAATTGCG
CGCTTGGCGTAATCATGGTCATAGCTGTTTCCTGTGTGAAATTGTTATCC
GCTCACAATTCCACACAACATAGGAGCCGGAAGCATAAAGTGTAAAGCC
TGGGGTGCCTAATGAGTGAGGTAACTCACATTAATTGCGTTGCGCTCAC
TGCCCGCTTTCCAGTCGGGAAACCTGTCGTGCCAGCTGCATTAATGAAT
CGGCCAACGCGCGGGGAGAGGCGGTTTGCGTATTGGGCGCTCTTCCGC
TTCCTCGCTCACTGACTCGCTGCGCTCGGTCGTTCGGCTGCGGCGAGC
GGTATCAGCTCACTCAAAGGCGGTAATACGGTTATCCACAGAATCAGGG
GATAACGCAGGAAAGAACATGTGAGCAAAAGGCCAGCAAAAGGCCAGG
AACCGTAAAAAGGCCGCGTTGCTGGCGTTTTTCCATAGGCTCCGCCCCC
CTGACGAGCATCACAAAAATCGACGCTCAAGTCAGAGGTGGCGAAACCC
GACAGGACTATAAAGATACCAGGCGTTTCCCCCTGGAAGCTCCCTCGTG
CGCTCTCCTGTTCCGACCCTGCCGCTTACCGGATACCTGTCCGCCTTTC
TCCCTTCGGGAAGCGTGGCGCTTTCTCATAGCTCACGCTGTAGGTATCT
CAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGT
CCAACCCGGTAAGACACGACTTATCGCCACTGGCAGCAGCCACTGGTAA
CAGGATTAGCAGAGCGAGGTATGTAGGCGGTGCTACAGAGTTCTTGAAG
TGGTGGCCTAACTACGGCTACACTAGAAGGACAGTATTTGGTATCTGCG
CTCTGCTGAAGCCAGTTACCTTCGGAAAAAGAGTTGGTAGCTCTTGATCC
GGCAAACAAACCACCGCTGGTAGCGGTGGTTTTTTTGTTTGCAAGCAGC
AGATTACGCGCAGAAAAAAAGGATCTCAAGAAGATCCTTTGATCTTTTCT
ACGGGGTCTGACGCTCAGTGGAACGAAAACTCACGTTAAGGGATTTTGG
TCATGAGATTATCAAAAAGGATCTTCACCTAGATCCTTTTAAATTAAAAAT
GAAGTTTTAAATCAATCTAAAGTATATATGAGTAAACTTGGTCTGACAGTT
ACCAATGCTTAATCAGTGAGGCACCTATCTCAGCGATCTGTCTATTTCGT
TCATCCATAGTTGCCTGACTCCCCGTCGTGTAGATAACTACGATACGGG
AGGGCTTACCATCTGGCCCCAGTGCTGCAATGATACCGCGAGACCCAC
GCTCACCGGCTCCAGATTTATCAGCAATAAACCAGCCAGCCGGAAGGGC
CGAGCGCAGAAGTGGTCCTGCAACTTTATCCGCCTCCATCCAGTCTATT
AATTGTTGCCGGGAAGCTAGAGTAAGTAGTTCGCCAGTTAATAGTTTGCG
CAACGTTGTTGCCATTGCTACAGGCATCGTGGTGTCACGCTCGTCGTTT
GGTATGGCTTCATTCAGCTCCGGTTCCCAACGATCAAGGCGAGTTACAT
GATCCCCCATGTTGTGCAAAAAAGCGGTTAGCTCCTTCGGTCCTCCGAT
CGTTGTCAGAAGTAAGTTGGCCGCAGTGTTATCACTCATGGTTATGGCA
GCACTGCATAATTCTCTTACTGTCATGCCATCCGTAAGATGCTTTTCTGT
GACTGGTGAGTACTCAACCAAGTCATTCTGAGAATAGTGTATGCGGCGA
CCGAGTTGCTCTTGCCCGGCGTCAATACGGGATAATACCGCGCCACATA
GCAGAACTTTAAAAGTGCTCATCATTGGAAAACGTTCTTCGGGGCGAAAA
CTCTCAAGGATCTTACCGCTGTTGAGATCCAGTTCGATGTAACCCACTCG
TGCACCCAACTGATCTTCAGCATCTTTTACTTTCACCAGCGTTTCTGGGT
GAGCAAAAACAGGAAGGCAAAATGCCGCAAAAAAGGGAATAAGGGCGA
CACGGAAATGTTGAATACTCATACTCTTCCTTTTTCAATATTATTGAAGCA
TTTATCAGGGTTATTGTCTCATGAGCGGATACATATTTGAATGTATTTAGA
AAAATAAACAAATAGGGGTTCCGCGCACATTTCCCCGAAAAGTGCCACC
TGAACGAAGCATCTGTGCTTCATTTTGTAGAACAAAAATGCAACGCGAGA
GCGCTAATTTTTCAAACAAAGAATCTGAGCTGCATTTTTACAGAACAGAA
ATGCAACGCGAAAGCGCTATTTTACCAACGAAGAATCTGTGCTTCATTTT
TGTAAAACAAAAATGCAACGCGAGAGCGCTAATTTTTCAAACAAAGAATC
TGAGCTGCATTTTTACAGAACAGAAATGCAACGCGAGAGCGCTATTTTAC
CAACAAAGAATCTATACTTCTTTTTTGTTCTACAAAAATGCATCCCGAGAG
CGCTATTTTTCTAACAAAGCATCTTAGATTACTTTTTTTCTCCTTTGTGCG
CTCTATAATGCAGTCTCTTGATAACTTTTTGCACTGTAGGTCCGTTAAGGT
TAGAAGAAGGCTACTTTGGTGTCTATTTTCTCTTCCATAAAAAAAGCCTGA
CTCCACTTCCCGCGTTTACTGATTACTAGCGAAGCTGCGGGTGCATTTTT
TCAAGATAAAGGCATCCCCGATTATATTCTATACCGATGTGGATTGCGCA
TACTTTGTGAACAGAAAGTGATAGCGTTGATGATTCTTCATTGGTCAGAA
AATTATGAACGGTTTCTTCTATTTTGTCTCTATATACTACGTATAGGAAAT
GTTTACATTTTCGTATTGTTTTCGATTCACTCTATGAATAGTTCTTACTACA
ATTTTTTTGTCTAAAGAGTAATACTAGAGATAAACATAAAAAATGTAGAGG
TCGAGTTTAGATGCAAGTTCAAGGAGCGAAAGGTGGATGGGTAGGTTAT
ATAGGGATATAGCACAGAGATATATAGCAAAGAGATACTTTTGAGCAATG
TTTGTGGAAGCGGTATTCGCAATATTTTAGTAGCTCGTTACAGTCCGGTG
CGTTTTTGGTTTTTTGAAAGTGCGTCTTCAGAGCGCTTTTGGTTTTCAAAA
GCGCTCTGAAGTTCCTATACTTTCTAGAGAATAGGAACTTCGGAATAGGA
ACTTCAAAGCGTTTCCGAAAACGAGCGCTTCCGAAAATGCAACGCGAGC
TGCGCACATACAGCTCACTGTTCACGTCGCACCTATATCTGCGTGTTGC
CTGTATATATATATACATGAGAAGAACGGCATAGTGCGTGTTTATGCTTA
AATGCGTACTTATATGCGTCTATTTATGTAGGATGAAAGGTAGTCTAGTA
CCTCCTGTGATATTATCCCATTCCATGCGGGGTATCGTATGCTTCCTTCA
GCACTACCCTTTAGCTGTTCTATATGCTGCCACTCCTCAATTGGATTAGT
CTCATCCTTCAATGCTATCATTTCCTTTGATATTGGATCATCTAAGAAACC
ATTATTATCATGACATTAACCTATAAAAATAGGCGTATCACGAGGCCCTTT
CGTC
SEQ ID 45: TRP1::RPR1-RNAi
CGTCGACGGTATCGATAAGCTTGATGTGTGCCCAATAGAAAGAGAACAA
TTGACCCGGTTATTGCAAGGAAAATTTCAAGTCTTGTAAAAGCATATAAAA
ATAGTTCAGGCACTCCGAAATACTTGGTTGGCGTGTTTCGTAATCAACCT
AAGGAGGATGTTTTGGCTCTGGTCAATGATTACGGCATTGATATCGTCCA
ACTGCATGGAGCTCGGTACCCGAGTTAAAGATCTGCCAATTGAACATAA
CATGGTAGTTACATATACTAGTAATATGGTTCGGCACACATTAAAAGTATA
AAAACTATCTGAATTACGAATTACATATATTGGTCATAAAAATCAATCAAT
CATCGTGTGTTTTATATGTCTCTTATCTAAGTATAAGAATATCCATAGTTA
ATATTCACTTACGCTACCTTTTAACCTGTAATCATTGTCAACAGGATATGT
TAACGACCCACATTGATAAACGCTAGTATTTCTTTTTCCTCTTCTTATTGG
CCGGCTGTCTCTATACTCCCCTATAGTCTGTTTCTTTTCGTTTCGATTGTT
TTACGTTTGAGGCCTCGTGGCGCACATGGTACGCTGTGGTGCTCGCGG
CTGGGAACGAAACTCTGGGAGCTGCGATTGGCAGCAATCTAATCTAAGT
CTAGAACGCTAAGTCGGAGGACGGACGGTCAGGTACTAGCGGCGGTGT
CTAGTTTGCTCTTGCCATCAACAATGCGTGCCATGCCTTTTCTCGAATGT
ATTTTACAATTTCTGAAGACGTCGGGATTGGAAATCCCAAAGTATTAATAA
GCACATTGTTTATAAGACTCGCATGTATGTTAATACTGTGGATCCGTGAG
TTTCTATTCGCAGTCGGCTGATCTGTGTGAAATCTTAATAAAGGGTCCAA
TTACCAATTTGAAACTCAGGAATTCACAGTATTAACATACATGCGAGTCTT
ATAAACAATGTGCTTATTAATACTTTGGGATTTCCAATCCCGACGTCTTCA
GAAATTGTAAAATACATTCGAGAAAAGGCATGGCACGCATTGTTGATGGC
AAGAGCAAACTAGACACCGCCGCTAGTACCTGACCGTCCGTCCTCCGAC
TTAGCGTAAGCTTTCATGTCCATATCCAACTTCCAATTTAATCTTTCTTTTT
TAATTTTCACTTATTTGCGATACAGAAAGAGGGGATCCGACATGGAGGCC
CAGAATACCCTCCTTGACAGTCTTGACGTGCGCAGCTCAGGGGCATGAT
GTGACTGTCGCCCGTACATTTAGCCCATACATCCCCATGTATAATCATTT
GCATCCATACATTTTGATGGCCGCACGGCGCGAAGCAAAAATTACGGCT
CCTCGCTGCAGACCTGCGAGCAGGGAAACGCTCCCCTCACAGACGCGT
TGAATTGTCCCCACGCCGCGCCCCTGTAGAGAAATATAAAAGGTTAGGA
TTTGCCACTGAGGTTCTTCTTTCATATACTTCCTTTTAAAATCTTGCTAGG
ATACAGTTCTCACATCACATCCGAACATAAACAACCATGGGTACCACTCT
TGACGACACGGCTTACCGGTACCGCACCAGTGTCCCGGGGGACGCCGA
GGCCATCGAGGCACTGGATGGGTCCTTCACCACCGACACCGTCTTCCG
CGTCACCGCCACCGGGGACGGCTTCACCCTGCGGGAGGTGCCGGTGG
ACCCGCCCCTGACCAAGGTGTTCCCCGACGACGAATCGGACGACGAAT
CGGACGACGGGGAGGACGGCGACCCGGACTCCCGGACGTTCGTCGCG
TACGGGGACGACGGCGACCTGGCGGGCTTCGTGGTCATCTCGTACTCG
GCGTGGAACCGCCGGCTGACCGTCGAGGACATCGAGGTCGCCCCGGA
GCACCGGGGGCACGGGGTCGGGCGCGCGTTGATGGGGCTCGCGACGG
AGTTCGCCGGCGAGCGGGGCGCCGGGCACCTCTGGCTGGAGGTCACC
AACGTCAACGCACCGGCGATCCACGCGTACCGGCGGATGGGGTTCACC
CTCTGCGGCCTGGACACCGCCCTGTACGACGGCACCGCCTCGGACGGC
GAGCGGCAGGCGCTCTACATGAGCATGCCCTGCCCCTAATCAGTACTGA
CAATAAAAAGATTCTTGTTTTCAAGAACTTGTCATTTGTATAGTTTTTTTAT
ATTGTAGTTGTTCTATTTTAATCAAATGTTAGCGTGATTTATATTTTTTTTC
GCCTCGACATCATCTGCCCAGATGCGAAGTTAAGTGCGCAGAAAGTAAT
ATCATGCGTCAATCGTATGTGAATGCTGGTCGCTATACTGCAAGAATACC
AAGAGTTCCTCGGTTTGCCAGTTATTAAAAGACTCGTATTTCCAAAAGAC
TGCAACATACTACTCAGTGCAGCTTCACAGAAACCTCATTCGTTTATTCC
CTTGTTTGATTCAGAAGCAGGTGGGACAGGTGAACTTTTGGATTGGAACT
CGATTTCTGACTGGGTTGGAAGGCAAGAG
SEQ ID 46: pRS343-Psnr33TUB
TCGCGCGTTTCGGTGATGACGGTGAAAACCTCTGACACATGCAGCTCCC
GGAGACGGTCACAGCTTGTCTGTAAGCGGATGCCGGGAGCAGACAAGC
CCGTCAGGGCGCGTCAGCGGGTGTTGGCGGGTGTCGGGGCTGGCTTA
ACTATGCGGCATCAGAGCAGATTGTACTGAGAGTGCACCATAGACATGG
AGGCCCAGAATACCCTCCTTGACAGTCTTGACGTGCGCAGCTCAGGGG
CATGATGTGACTGTCGCCCGTACATTTAGCCCATACATCCCCATGTATAA
TCATTTGCATCCATACATTTTGATGGCCGCACGGCGCGAAGCAAAAATTA
CGGCTCCTCGCTGCAGACCTGCGAGCAGGGAAACGCTCCCCTCACAGA
CGCGTTGAATTGTCCCCACGCCGCGCCCCTGTAGAGAAATATAAAAGGT
TAGGATTTGCCACTGAGGTTCTTCTTTCATATACTTCCTTTTAAAATCTTG
CTAGGATACAGTTCTCACATCACATCCGAACATAAACAACCATGGGTAAG
GAAAAGACTCACGTTTCGAGGCCGCGATTAAATTCCAACATGGATGCTG
ATTTATATGGGTATAAATGGGCTCGCGATAATGTCGGGCAATCAGGTGC
GACAATCTATCGATTGTATGGGAAGCCCGATGCGCCAGAGTTGTTTCTG
AAACATGGCAAAGGTAGCGTTGCCAATGATGTTACAGATGAGATGGTCA
GACTAAACTGGCTGACGGAATTTATGCCTCTTCCGACCATCAAGCATTTT
ATCCGTACTCCTGATGATGCATGGTTACTCACCACTGCGATCCCCGGCA
AAACAGCATTCCAGGTATTAGAAGAATATCCTGATTCAGGTGAAAATATT
GTTGATGCGCTGGCAGTGTTCCTGCGCCGGTTGCATTCGATTCCTGTTT
GTAATTGTCCTTTTAACAGCGATCGCGTATTTCGTCTCGCTCAGGCGCAA
TCACGAATGAATAACGGTTTGGTTGATGCGAGTGATTTTGATGACGAGC
GTAATGGCTGGCCTGTTGAACAAGTCTGGAAAGAAATGCATAAGCTTTTG
CCATTCTCACCGGATTCAGTCGTCACTCATGGTGATTTCTCACTTGATAA
CCTTATTTTTGACGAGGGGAAATTAATAGGTTGTATTGATGTTGGACGAG
TCGGAATCGCAGACCGATACCAGGATCTTGCCATCCTATGGAACTGCCT
CGGTGAGTTTTCTCCTTCATTACAGAAACGGCTTTTTCAAAAATATGGTAT
TGATAATCCTGATATGAATAAATTGCAGTTTCATTTGATGCTCGATGAGTT
TTTCTAATCAGTACTGACAATAAAAAGATTCTTGTTTTCAAGAACTTGTCA
TTTGTATAGTTTTTTTATATTGTAGTTGTTCTATTTTAATCAAATGTTAGCG
TGATTTATATTTTTTTTCGCCTCGACATCATCTGCCCAGATGCGAAGTTAA
GTGCGCAGAAAGTAATATCATGCGTCAATCGTATGTGAATGCTGGTCGC
TATACTGTATGCGGTGTGAAATACCGCACAGATGCGTAAGGAGAAAATA
CCGCATCAGGAAATTGTAAACGTTAATATTTTGTTAAAATTCGCGTTAAAT
TTTTGTTAAATCAGCTCATTTTTTAACCAATAGGCCGAAATCGGCAAAATC
CCTTATAAATCAAAAGAATAGACCGAGATAGGGTTGAGTGTTGTTCCAGT
TTGGAACAAGAGTCCACTATTAAAGAACGTGGACTCCAACGTCAAAGGG
CGAAAAACCGTCTATCAGGGCGATGGCCCACTACGTGAACCATCACCCT
AATCAAGTTTTTTGGGGTCGAGGTGCCGTAAAGCACTAAATCGGAACCC
TAAAGGGAGCCCCCGATTTAGAGCTTGACGGGGAAAGCCGGCGAACGT
GGCGAGAAAGGAAGGGAAGAAAGCGAAAGGAGCGGGCGCTAGGGCGC
TGGCAAGTGTAGCGGTCACGCTGCGCGTAACCACCACACCCGCCGCGC
TTAATGCGCCGCTACAGGGCGCGTCGCGCCATTCGCCATTCAGGCTGC
GCAACTGTTGGGAAGGGCGATCGGTGCGGGCCTCTTCGCTATTACGCC
AGCTGGCGAAAGGGGGATGTGCTGCAAGGCGATTAAGTTGGGTAACGC
CAGGGTTTTCCCAGTCACGACGTTGTAAAACGACGGCCAGTGAGCGCG
CGTAATACGACTCACTATAGGGCGAATTGGGTACCGGGCCcccggttcgattcc
gggcttgcgcatcttttttactttatatactattttttttttttttctttttcccaaattttttcatgaaaaat
ttggcggaacggtacataagaatagaagagattcgttatgaaaattttctactctctttcacattttttttttc
ataagaattaaaaaaattCTAGAACGCTAAGTCGGAGGACGGACGGTCAGGTACTAGCGGCGGT
GTCTAGTTTGCTCTTGCCATCAACAATGCGTGCCATGCCTTTTCTCGAAT
GTATTTTACAATTTCTGAAGACGTCGGGATTGGAAATCCCAAAGTATTAAT
AAGCACATTGTTTATAAGACTCGCATGTATGTTAATACTGTGGATCCGTG
AGTTTCTATTCGCAGTCGGCTGATCTGTGTGAAATCTTAATAAAGGGTCC
AATTACCAATTTGAAACTCAGGAATTCACAGTATTAACATACATGCGAGTC
TTATAAACAATGTGCTTATTAATACTTTGGGATTTCCAATCCCGACGTCTT
CAGAAATTGTAAAATACATTCGAGAAAAGGCATGGCACGCATTGTTGATG
GCAAGAGCAAACTAGACACCGCCGCTAGTACCTGACCGTCCGTCCTCCG
ACTTAGCGTAAGCTTTCATGTAATTAGTTATGTCACGCTTACATTCACGCC
CTCCCCCCACATCCGCTCTAACCGAAAAGGAAGGAGTTAGACAACCTGA
AGTCTAGGTCCCTATTTATTTTTTTATAGTTATGTTAGTATTAAGAACGTTA
TTTATATTTCAAATTTTTCTTTTTTTTCTGTACAGACGCGTGTACGCATGTA
ACATTATACTGAAAACCTTGCTTGAGAAGGTTTTGGGACGCTCGAAGGCT
TTAATTTGCGTCGACGGTATCGATAAGCTTGATATCGAATTCCTGCAGCC
CGGGGGATCCACTAGTTCTAGAGCGGCCGCCACCGCGGTGGAGCTCCA
GCTTTTGTTCCCTTTAGTGAGGGTTAATTGCGCGCTTGGCGTAATCATGG
TCATAGCTGTTTCCTGTGTGAAATTGTTATCCGCTCACAATTCCACACAA
CATAGGAGCCGGAAGCATAAAGTGTAAAGCCTGGGGTGCCTAATGAGTG
AGGTAACTCACATTAATTGCGTTGCGCTCACTGCCCGCTTTCCAGTCGG
GAAACCTGTCGTGCCAGCTGCATTAATGAATCGGCCAACGCGCGGGGA
GAGGCGGTTTGCGTATTGGGCGCTCTTCCGCTTCCTCGCTCACTGACTC
GCTGCGCTCGGTCGTTCGGCTGCGGCGAGCGGTATCAGCTCACTCAAA
GGCGGTAATACGGTTATCCACAGAATCAGGGGATAACGCAGGAAAGAAC
ATGTGAGCAAAAGGCCAGCAAAAGGCCAGGAACCGTAAAAAGGCCGCG
TTGCTGGCGTTTTTCCATAGGCTCCGCCCCCCTGACGAGCATCACAAAA
ATCGACGCTCAAGTCAGAGGTGGCGAAACCCGACAGGACTATAAAGATA
CCAGGCGTTTCCCCCTGGAAGCTCCCTCGTGCGCTCTCCTGTTCCGACC
CTGCCGCTTACCGGATACCTGTCCGCCTTTCTCCCTTCGGGAAGCGTGG
CGCTTTCTCATAGCTCACGCTGTAGGTATCTCAGTTCGGTGTAGGTCGTT
CGCTCCAAGCTGGGCTGTGTGCACGAACCCCCCGTTCAGCCCGACCGC
TGCGCCTTATCCGGTAACTATCGTCTTGAGTCCAACCCGGTAAGACACG
ACTTATCGCCACTGGCAGCAGCCACTGGTAACAGGATTAGCAGAGCGAG
GTATGTAGGCGGTGCTACAGAGTTCTTGAAGTGGTGGCCTAACTACGGC
TACACTAGAAGGACAGTATTTGGTATCTGCGCTCTGCTGAAGCCAGTTAC
CTTCGGAAAAAGAGTTGGTAGCTCTTGATCCGGCAAACAAACCACCGCT
GGTAGCGGTGGTTTTTTTGTTTGCAAGCAGCAGATTACGCGCAGAAAAA
AAGGATCTCAAGAAGATCCTTTGATCTTTTCTACGGGGTCTGACGCTCAG
TGGAACGAAAACTCACGTTAAGGGATTTTGGTCATGAGATTATCAAAAAG
GATCTTCACCTAGATCCTTTTAAATTAAAAATGAAGTTTTAAATCAATCTAA
AGTATATATGAGTAAACTTGGTCTGACAGTTACCAATGCTTAATCAGTGA
GGCACCTATCTCAGCGATCTGTCTATTTCGTTCATCCATAGTTGCCTGAC
TCCCCGTCGTGTAGATAACTACGATACGGGAGGGCTTACCATCTGGCCC
CAGTGCTGCAATGATACCGCGAGACCCACGCTCACCGGCTCCAGATTTA
TCAGCAATAAACCAGCCAGCCGGAAGGGCCGAGCGCAGAAGTGGTCCT
GCAACTTTATCCGCCTCCATCCAGTCTATTAATTGTTGCCGGGAAGCTAG
AGTAAGTAGTTCGCCAGTTAATAGTTTGCGCAACGTTGTTGCCATTGCTA
CAGGCATCGTGGTGTCACGCTCGTCGTTTGGTATGGCTTCATTCAGCTC
CGGTTCCCAACGATCAAGGCGAGTTACATGATCCCCCATGTTGTGCAAA
AAAGCGGTTAGCTCCTTCGGTCCTCCGATCGTTGTCAGAAGTAAGTTGG
CCGCAGTGTTATCACTCATGGTTATGGCAGCACTGCATAATTCTCTTACT
GTCATGCCATCCGTAAGATGCTTTTCTGTGACTGGTGAGTACTCAACCAA
GTCATTCTGAGAATAGTGTATGCGGCGACCGAGTTGCTCTTGCCCGGCG
TCAATACGGGATAATACCGCGCCACATAGCAGAACTTTAAAAGTGCTCAT
CATTGGAAAACGTTCTTCGGGGCGAAAACTCTCAAGGATCTTACCGCTG
TTGAGATCCAGTTCGATGTAACCCACTCGTGCACCCAACTGATCTTCAGC
ATCTTTTACTTTCACCAGCGTTTCTGGGTGAGCAAAAACAGGAAGGCAAA
ATGCCGCAAAAAAGGGAATAAGGGCGACACGGAAATGTTGAATACTCAT
ACTCTTCCTTTTTCAATATTATTGAAGCATTTATCAGGGTTATTGTCTCAT
GAGCGGATACATATTTGAATGTATTTAGAAAAATAAACAAATAGGGGTTC
CGCGCACATTTCCCCGAAAAGTGCCACCTGAACGAAGCATCTGTGCTTC
ATTTTGTAGAACAAAAATGCAACGCGAGAGCGCTAATTTTTCAAACAAAG
AATCTGAGCTGCATTTTTACAGAACAGAAATGCAACGCGAAAGCGCTATT
TTACCAACGAAGAATCTGTGCTTCATTTTTGTAAAACAAAAATGCAACGC
GAGAGCGCTAATTTTTCAAACAAAGAATCTGAGCTGCATTTTTACAGAAC
AGAAATGCAACGCGAGAGCGCTATTTTACCAACAAAGAATCTATACTTCT
TTTTTGTTCTACAAAAATGCATCCCGAGAGCGCTATTTTTCTAACAAAGCA
TCTTAGATTACTTTTTTTCTCCTTTGTGCGCTCTATAATGCAGTCTCTTGA
TAACTTTTTGCACTGTAGGTCCGTTAAGGTTAGAAGAAGGCTACTTTGGT
GTCTATTTTCTCTTCCATAAAAAAAGCCTGACTCCACTTCCCGCGTTTACT
GATTACTAGCGAAGCTGCGGGTGCATTTTTTCAAGATAAAGGCATCCCC
GATTATATTCTATACCGATGTGGATTGCGCATACTTTGTGAACAGAAAGT
GATAGCGTTGATGATTCTTCATTGGTCAGAAAATTATGAACGGTTTCTTCT
ATTTTGTCTCTATATACTACGTATAGGAAATGTTTACATTTTCGTATTGTTT
TCGATTCACTCTATGAATAGTTCTTACTACAATTTTTTTGTCTAAAGAGTAA
TACTAGAGATAAACATAAAAAATGTAGAGGTCGAGTTTAGATGCAAGTTC
AAGGAGCGAAAGGTGGATGGGTAGGTTATATAGGGATATAGCACAGAGA
TATATAGCAAAGAGATACTTTTGAGCAATGTTTGTGGAAGCGGTATTCGC
AATATTTTAGTAGCTCGTTACAGTCCGGTGCGTTTTTGGTTTTTTGAAAGT
GCGTCTTCAGAGCGCTTTTGGTTTTCAAAAGCGCTCTGAAGTTCCTATAC
TTTCTAGAGAATAGGAACTTCGGAATAGGAACTTCAAAGCGTTTCCGAAA
ACGAGCGCTTCCGAAAATGCAACGCGAGCTGCGCACATACAGCTCACTG
TTCACGTCGCACCTATATCTGCGTGTTGCCTGTATATATATATACATGAG
AAGAACGGCATAGTGCGTGTTTATGCTTAAATGCGTACTTATATGCGTCT
ATTTATGTAGGATGAAAGGTAGTCTAGTACCTCCTGTGATATTATCCCATT
CCATGCGGGGTATCGTATGCTTCCTTCAGCACTACCCTTTAGCTGTTCTA
TATGCTGCCACTCCTCAATTGGATTAGTCTCATCCTTCAATGCTATCATTT
CCTTTGATATTGGATCATCTAAGAAACCATTATTATCATGACATTAACCTA
TAAAAATAGGCGTATCACGAGGCCCTTTCGTC

### REFERENCES

Anderson, K. E., Sheehan, T. H., Eckholm, B. J., & Mott, B. M. (2011). An emerging paradigm of colony health: microbial balance of the honey bee and hive (Apis mellifera). Insectes Sociaux 58:431.
Atwood, D., & Paisley, C. (2017). Pesticides industry sales and usage 2008-2012 market estimates. United States Environmental Protection Agency. https://www.epa.gov/sites/production/files/2017-01/documents/pesticides-industry-sales-usage-2016_0.pdf
Beketov, M. A., & Kefford, B. J. (2013). Pesticides reduce regional biodiversity of stream invertebrates. Proceedings of the National Academy of Sciences, 110(27), 11039. http://doi.org/10.1073/pnas.1305618110.
Bradford, B. J., Cooper, C. A., Tizard, M. L., & Doran, T. J. (2017). RNA interference-based technology: what role in animal agriculture? Animal Production Science, 57(1), 1. http://doi.org/10.1071/an15437.
Butler, D. (2010). Food: the growing problem. Nature. 466: 546-547.
Chang, Q., Wang, W., Regev-Yochay, G., Lipsitch, M., & Hanage, W. P. (2014). Antibiotics in agriculture and the risk to human health: how worried should we be? Evolutionary Applications, 8(3), 240-247. http://doi.org/10.1111/eva.12185
Coccia, M., et al., IL-1beta mediates chronic intestinal inflammation by promoting the accumulation of IL-17A secreting innate lymphoid cells and CD4(+) Th17 cells. J Exp Med, 2012. 209(9): p. 1595-609.
Connolly, B., Isaacs, C., Cheng, L., Asrani, K. H., & Subramanian, R. R. (2018). SERPINA1 mRNA as a Treatment for Alpha-1 Antitrypsin Deficiency. Journal of nucleic acids, 2018.
DiCarlo, J. E., Norville, J. E., Mali, P., Rios, X., Aach, J., & Church, G. M. (2013). Genome engineering in Saccharomyces cerevisiae using CRISPR-Cas systems. Nucleic acids research, 41(7), 4336-4343.
Drummond, R. O., Lambert, G., Smalley Jr, H. E., & Terrill, C. E. (1981). Estimated losses of livestock to pests [USA]. CRC Handbook of Pest Management in Agriculture (USA).
Duman-Scheel, M., Eggleson, K. K., Achee, N. L., Grieco, J. P. & Hapairai, L. K. Mosquito control practices and perceptions: An analysis of economic stakeholders during the Zika epidemic in Belize, Central America. PloS One 13, e0201075 (2018).
Fire, A., Kostas, S., Montgomery, M., Timmons, L., Xu, S., Tabara, H., . . . & Mello, C. C. (2003). U.S. Pat. No. 6,506,559. Washington, DC: U.S. Patent and Trademark Office.
Fujita, T., Ikuta, J., Hamada, J., Okajima, T., Tatematsu, K., Tanizawa, K., & Kuroda, S. I. (2004). Identification of a tissue-non-specific homologue of axonal fasciculation and elongation protein zeta-1. Biochemical and biophysical research communications, 313(3), 738-744.
Garcia, J. F., Carbone, M. A., Mackay, T. F., & Anholt, R. R. (2017). Regulation of Drosophila Lifespan by bellwether Promoter Alleles. Scientific reports, 7(1), 4109.
Ghosh, S., Hunter, W. B., & Park, A. L. (2017). Double strand RNA delivery system for plant-sap-feeding insects. PLOS ONE, 12(2), e0171861. http://doi.org/10.1371/journal.pone.0171861.
Gietz, R. D., & Schiestl, R. H. (2007). High-efficiency yeast transformation using the LiAc/SS carrier DNA/PEG method. Nature protocols, 2(1), 31.
Giraldo-Calderon, Gloria I., et al. “VectorBase: an updated bioinformatics resource for invertebrate vectors and other organisms related with human diseases.” Nucleic acids research 43.D1 (2014): D707-D713.
Hapairai, L. K. et al. Lure-and-Kill Yeast Interfering RNA Larvicides Targeting Neural Genes in the Human Disease Vector Mosquito Aedes aegypti. Sci. Rep. 7, 13223 (2017).
Jacobs, H., R. Stratmann, and C. F. Lehner. “A screen for lethal mutations in the chromosomal region 59AB suggests that bellwether encodes the alpha subunit of the mitochondrial ATP synthase in Drosophila melanogaster.” Molecular and General Genetics MGG 259.4 (1998): 383-387.
Jin, S., Singh, N. D., Li, L., Zhang, X., & Daniell, H. (2015). Engineered chloroplast dsRNA silences cytochrome p450 monooxygenase, V-ATPase and chitin synthase genes in the insect gut and disrupts Helicoverpa armigera larval development and pupation. Plant biotechnology journal, 13(3), 435-446.
Joga, M. R., Zotti, M. J., Smagghe, G., & Christiaens, O. (2016). RNAi efficiency, systemic properties, and novel delivery methods for pest insect control: what we know so far. Frontiers in physiology, 7, 553.
Köhrer, K., & Domdey, H. (1991). [27] Preparation of high molecular weight RNA. In Methods in enzymology (Vol. 194, pp. 398-405). Academic Press.
Laroui H., Theiss A L., Yan Y., Dalmasso G., Nguyen H T T., Sitaraman, S V., & Merlin, D. (2011). Functional TNFα gene silencing mediated by polyethyleneimine/TNFα siRNA nanocomplexes in inflamed colon. Biomaterials 32(4):1218-1228.
Li, X., Zhang, M., & Zhang, H. (2011). RNA interference of four genes in adult Bactrocera dorsalis by feeding their dsRNAs. PLOS ONE, 6(3), e17788. http://doi.org/10.1371/journal.pone.0017788.
Lin, Y. H., Huang, J. H., Liu, Y., Belles, X., & Lee, H. J. (2017). Oral delivery of dsRNA lipoplexes to German cockroach protects dsRNA from degradation and induces RNAi response. Pest management science, 73(5), 960-966.
Lopez, S. B. G. et al. RNAi-based bioinsecticide for Aedes mosquito control. Sci. Rep. 9, 4038 (2019).
Lu, H. L., Vinson, S. B., & Pietrantonio, P. V. (2009). Oocyte membrane localization of vitellogenin receptor coincides with queen flying age, and receptor silencing by RNAi disrupts egg formation in fire ant virgin queens. The FEBS journal, 276(11), 3110-3123.
McKinnon, M. L., et al., Combined immunodeficiency associated with homozygous MALT1 mutations. J Allergy Clin Immunol, 2014. 133(5): p. 1458-62, 1462 e1-7.
McLarren, K. W., et al., SHIP-deficient mice develop spontaneous intestinal inflammation and arginase-dependent fibrosis. Am J Pathol, 2011. 179(1): p. 180-8.
Monajemi, M., et al., Malt1 blocks IL-1beta production by macrophages in vitro and limits dextran sodium sulfate-induced intestinal inflammation in vivo. J Leukoc Biol, 2018. 104(3): p. 557-572.
Murphy, K. A. et al. (2016) Ingestion of genetically modified yeast symbiont reduces fitness of an insect pest via RNA interference. Sci Rep-uk 6, 1 13
Mysore, Keshava, et al. “Yeast interfering RNA larvicides targeting neural genes induce high rates of Anopheles larval mortality.” Malaria journal 16.1 (2017): 461.
Mysore, Keshava, et al. “Preparation and Use of a Yeast shRNA Delivery System for Gene Silencing in Mosquito Larvae.” Insect Genomics. Humana Press, New York, N.Y., 2019. 213-231.
Ngoh, E. N., et al., Activity of SHIP, Which Prevents Expression of Interleukin 1beta, Is Reduced in Patients With Crohn's Disease. Gastroenterology, 2016. 150(2): p. 465-76.
Oerke, E. C. (2006). Crop losses to pests. The Journal of Agricultural Science, 144(1), 31-43.
Pagel, S. W., & Gautier, P. (2012). Use of antimicrobial agents in livestock. Revue Scientifique Et Technique (International Office of Epizootics), 31(1), 145-188.
Pimentel, D., & Burgess, M. (2014). Environmental and economic costs of the application of pesticides primarily in the United States. In Integrated pest management (pp. 47-71). Springer, Dordrecht.
Pimentel, David, and Michael Burgess. “Small amounts of pesticides reaching target insects.” (2012): 1-2.
Prieve, M. G., Harvie, P., Monahan, S. D., Roy, D., Li, A. G., Blevins, T. L., &
Ella-Menye, J. R. (2018). Targeted mRNA therapy for ornithine transcarbamylase deficiency. Molecular Therapy, 26(3), 801-813.
Roseman, Daniel S., et al. “G6PC mRNA therapy positively regulates fasting blood glucose and decreases liver abnormalities in a mouse model of glycogen storage disease 1 a.” Molecular Therapy 26.3 (2018): 814-821.
Tiemann, K. & Rossi JJ. (2009). RNAi-based therapeutics-current status, challenges and prospects. EMBO Molecular Medicine 1:142-151.
Trepotec, Z., Lichtenegger, E., Plank, C., Aneja, M. K., & Rudolph, C. (2018). Delivery of mRNA therapeutics for treatment of hepatic diseases. Molecular Therapy. 27(4): 794-802.
Van Boeckel, T. P., Brower, C., & Gilbert, M. (2015). Global trends in antimicrobial use in food animals. Proceedings of the National Academy of Sciences, 112(18), 5649. http://doi.org/10.1073/pnas.1503141112
Whitten, M., Facey, P. D., & Del Sol, R. (2016). Symbiont-mediated RNA interference in insects. Proceedings of the Royal Society B: Biological Sciences, 283(1825), 20160042. http://doi.org/10.1098/rspb.2016.0042
Whyard, S., Singh, A. D. & Wong, S. Ingested double-stranded RNAs can act as species-specific insecticides. Insect Biochem. Mol. Biol. 39, 824-832 (2009).
Winzeler, Elizabeth A., et al. “Functional characterization of the S. cerevisiae genome by gene deletion and parallel analysis.” Science 285.5429 (1999): 901-906.
Xiong, Q., Lee, G. Y., Ding, J., Li, W., & Shi, J. (2018). Biomedical applications of mRNA nanomedicine. Nano research, 11(10), 5281-5309.
Yu, Xiu-Dao, et al. “RNAi-mediated plant protection against aphids.” Pest management science 72.6 (2016): 1090-1098.

1. A yeast cell comprising an RNA instability gene(s) that is downregulated or inactivated and/or an RNA stability gene(s) that is upregulated or heterologously expressed; and at least one heterologous sequence that encodes an RNA bioactive molecule.

2. The yeast cell of claim 1, wherein the at least one heterologous sequence that encodes the RNA bioactive molecule is integrated into the yeast genome or is plasmid-based.

3. The yeast cell of claim 1, wherein the yeast is from Saccharomyces, optionally S. cerevisiae.

4. The yeast cell of claim 1, wherein the yeast comprises an RNA stability gene(s) that is upregulated or heterologously expressed and wherein the RNA stability gene is contained in an expression cassette that is integrated into the yeast genome or is plasmid-based and/or wherein the yeast comprises an RNA instability gene and wherein the RNA instability gene is downregulated or inactivated due to a deletion or inactivation of the RNA instability gene.

5. The yeast cell of claim 1, wherein the RNA stability gene that is upregulated or heterologously expressed is CCR4, THP1, XRN1 or TAF1.

6. The yeast cell of claim 1, wherein the RNA instability gene that is downregulated or inactivated comprises APN1, DBR1, DCS1, EDC3, HBS1, HTZ1, IPK1, LRP1, MAK10, MAK3, MAK31, MKT1, MPP6, MRT4, NAM7, NMD2, PAP2, POP2, RNH1, RNH203, RPS28A, RRP6, SIR3, SKI2, SKI3, SKI7, SKIS, SLH1, TRF5, or UPF3.

7. The yeast cell of claim 1, wherein the RNA instability gene is RRP6.

8. The yeast cell of claim 1, wherein the RNA instability gene is SKI3.

9. The yeast cell of claim 1, wherein two RNA instability genes are downregulated or inactivated.

10. The yeast cell of claim 9, wherein the two RNA instability genes are RRP6 and SKI3.

11. The yeast cell of claim 1, wherein the RNA bioactive molecule is an mRNA molecule that encodes a protein that is useful for the treatment of a disease and/or infection, a protein that is related to a protein deficiency or a protein that can elicit an immune response for prevention or treatment of disease and/or infection.

12. The yeast cell of claim 1, wherein the RNA bioactive molecule is an RNAi effector molecule, wherein the RNAi effector molecule is siRNA, miRNA, lhRNA, shRNA, dsRNA, or anti-sense RNA.

13. The yeast cell of claim 12, wherein the RNAi effector molecule targets a gene involved in survival, maturation, reproduction, aggressiveness, or virulence of a pest, a parasite, a bacterium, a fungus, or a virus.

14. The yeast cell of claim 13, wherein the gene involved in survival, maturation, reproduction, aggressiveness, or virulence is actin, VATPase, cytochrome P450, hemolin, hunchback, bellwether, fez2, bicoid, modsp, boule, gas8, gnbpa1, gnpba3, tubulin, Sac1, Irc, otk, neurexin-IV or vitellogenin.

15. The yeast cell of 12, wherein the RNAi effector molecule targets a gene involved in promoting a disease state, wherein the gene involved in promoting a disease state is actin, VATPase, cytochrome p450, hemolin, hunchback, vitellogenin, VEGF, VEGFR1, DDIT4, KRT6A, RRM2, p53, LMP2, LMP7, MECL1, IL-1β, or TNF-α.

16. A method of producing the yeast cell of claim 1 comprising:
a) downregulating or inactivating the RNA instability gene(s) and/or upregulating or heterologously expressing the RNA stability gene(s); and
b) expressing at least one heterologous sequence that encodes the RNA bioactive molecule.

17. The method of claim 16, wherein downregulating or inactivating the RNA instability gene in a) comprises deleting the gene from the yeast genome and/or wherein b) comprises integrating the at least one heterologous sequence into the yeast genome or introducing at least one plasmid-based heterologous sequence.

18. A method of biocontrol comprising exposing an unwanted organism to the yeast cell of claim 1, wherein the RNA bioactive molecule reduces the survival, maturation, reproduction, aggressiveness, or virulence of the unwanted organism.

19. The method of claim 18, wherein the unwanted organism is a pest, a bacterium, a virus, a fungus, or a parasite.

20. The method of claim 18, wherein exposing the organism to the yeast cell comprises feeding the yeast cells to the unwanted organism or feeding the yeast cells to a host organism harboring the unwanted organism.

21. The method of claim 18, wherein the RNA bioactive molecule is an mRNA that encodes a toxic factor or a negative regulatory factor in a host harboring the unwanted organism or wherein the RNA bioactive molecule is an RNAi effector molecule that targets a gene in the unwanted organism that is responsible for survival, maturation, reproduction, aggressiveness, or virulence.

22. The method of claim 21, wherein the unwanted organism is an agricultural pest, such as an insect.

23. The method of claim 21, wherein the gene is actin, VATPase, cytochrome P450, hemolin, hunchback, bellwether, fez2, bicoid, modsp, boule, gas8, gnbpa1, gnpba3, tubulin, Sac1, Irc, otk, neurexin IV or vitellogenin.

24. A method of treating a disease or infection comprising exposing the yeast cell of claim 1 to a subject in need thereof, wherein the RNA bioactive molecule is an mRNA molecule that encodes a protein that is useful for the treatment of the disease or infection, a protein that is related to a protein deficiency or a protein that can elicit an immune response for prevention or treatment of the disease or infection, or wherein the RNA bioactive molecule is an RNAi effector molecule that targets a disease promoting gene or is an RNAi effector molecule that targets an organism causing the infection in the subject or that targets a host factor in the subject that promotes the infection in the subject.

25. The method of claim 24, wherein the yeast is exposed by administration orally, topically, intravenously, intradermally, intramuscularly, or subcutaneously.

26. The method of claim 24, wherein the subject is livestock, a companion animal, a plant or a human.

27. The method of claim 24, wherein the disease promoting gene is actin, VATPase, cytochrome p450, hemolin, hunchback, vitellogenin, VEGF, VEGFR1, DDIT4, KRT6A, RRM2, p53, LMP2, LMP7, MECL1, IL-1β or TNF-α.

28. The method of claim 24, wherein the organism causing the infection is a virus, parasite, a fungus, or a bacterium.
